(19) United States
(12) Patent Application Publication
(10) Pub. No.: US 2026/0318552 A1
(43) Pub. Date: Oct. 15, 2026

(54) AIR PURIFICATION AND MANAGEMENT SYSTEM FOR BUILDINGS AND METHOD

(71) Applicant: The Boeing Company, Arlington, VA (US)

(72) Inventors: Robert E. Grip, Rancho Palos Verdes, CA (US); Michael F. Stoia, Rancho Santa Margarita, CA (US); Zachary C. Hoisington, Seal Beach, CA (US); John J. Brown, Costa Mesa, CA (US)

(73) Assignee: The Boeing Company, Arlington, VA (US)

(21) Appl. No.: 19/227,258

(22) Filed: Jun. 3, 2025

Related U.S. Application Data
(60) Provisional application No. 63/724,310, filed on Nov. 23, 2024.

(51) Int. Cl. F24F 8/20; A61L 9/16

(52) U.S. Cl. CPC F24F 8/20; A61L 9/16

(57) ABSTRACT

There is provided an air purification and management system for building(s), having an air purification system, an air management system, and control system(s). The air purification system includes at least one compressor assembly having: an air intake assembly to take in one or more of, external air, and recirculated air; at least one compressor to compress one or more of, the external air, and the recirculated air, to obtain compressed air; at least one plenum to purify the compressed air by deactivating one or more of, one or more biological agents, and one or more chemical agents, to obtain purified air; at least one decompressor; and an air outlet assembly. The air management system includes external air intake duct(s), an air supply duct assembly, and an air return duct assembly. The air purification and management system provides a contamination mitigation for occupants of the building(s) against contamination.

### CROSS-REFERENCE TO RELATED APPLICATION

This non-provisional application claims priority to pending U.S. Provisional Application Ser. No. 63/724,310, filed Nov. 23, 2024, which is incorporated herein by reference in its entirety.

### FIELD

The disclosure relates generally to airflow systems and methods for buildings, and more particularly, to air purification and management airflow systems and methods to provide contamination mitigation for occupants of buildings against biological and chemical agents.

### BACKGROUND

Pandemics, biological warfare using biological weapons, and chemical warfare using chemical weapons can cause harm or death to humans, and increasing attention has been given to systems and methods to prevent or mitigate such harm or death and to make buildings safe for human occupants. The recent COVID-19 (coronavirus disease 2019) pandemic revealed vulnerabilities in airflow systems, such as heating, ventilation, and air conditioning (HVAC) systems, in office buildings and commercial buildings, and concern that the air in office buildings and commercial buildings contains a virus that is not detectable remains an issue.

Known safety measures during and after the COVID-19 pandemic used by occupants in office buildings and commercial buildings included wearing face masks, social distancing, and sanitizing the work area. However, cloth face masks have been found not to be effective, and face masks that are more effective may be uncomfortable to wear. Further, effective face masks may be able to block coronavirus-carrying respiratory droplets, but may not be able to block smaller aerosol or airborne particles. In addition, sanitizing the work area, such as frequently wiping down surfaces, may be expensive and time consuming to implement and maintain, and such sanitizing does not address the quality and purification of the air in the work area and does not solve the spread of airborne viruses.

Another known safety measure used during and after the COVID-19 pandemic in office buildings and commercial buildings included the use of an air filter designed to trap and kill the coronavirus with heat. The coronavirus dies at 158 degrees Fahrenheit. Some air filters use a grid of wires heated to 392 degrees Fahrenheit. However, such a filter may have limited usefulness to coronavirus since the dwell time the air is heated to 392 degrees Fahrenheit may be insufficient to kill all the viruses passing through it. It may also not have the capability of heating to higher temperatures, e.g., above 392 degrees Fahrenheit, to kill other biological agents and chemical agents that can only be destroyed at higher temperatures. The combination of a limited temperature and a short dwell time results in a system that does not reduce the risk to near zero.

Another known safety measure used during and after the COVD-19 pandemic in office buildings and commercial spaces included the use of a conventional ultraviolet (UV) light in air duct systems to destroy coronavirus airborne droplets and other viruses and microbes on surfaces. However, the range of temperatures studied with such far conventional UV light is narrow, e.g., 74 degrees Fahrenheit to 95 degrees Fahrenheit. Moreover, such far conventional UV light does not reduce the risk to near zero. In addition, use of germicidal ultraviolet (UV) light can destroy airborne viruses but may damage human skin and eyes, and must be used in empty rooms with no human occupants. However, the germicidal UV light may be used inside air ducts.

Accordingly, there is a need in the art for an improved air purification and management system and method for buildings, such as office buildings and commercial buildings, that provide effective heating temperatures and effective dwell times to destroy numerous types of biological and chemical agents, that provide heating temperatures above 392 degrees Fahrenheit, that provide an easily adjustable maximum temperature, that enable the destruction of any biological agent or chemical agent by increasing heating temperatures to a maximum, that purify the air to near zero risk, that do not damage or harm human occupants of a building, that provide contamination mitigation for occupants of buildings against contamination by biological agents and chemical agents, that do not require any filters to change or maintain, and that provide advantages over known systems and methods.

### SUMMARY

Example implementations of the present disclosure provide an air purification and management system and method for one or more buildings. As discussed in the below detailed description, versions of the air purification and management system and method for one or more buildings may provide significant advantages over known systems and methods.

In a version of the disclosure, there is provided an air purification and management system for one or more buildings. The air purification and management system comprises an air purification system. The air purification system comprises at least one compressor assembly. The at least one compressor assembly comprises an air intake assembly configured to take in one or more of, external air external to the one or more buildings, the external air including external air contaminated with one or more of, one or more biological agents, and one or more chemical agents, and recirculated air from one or more interior airflow spaces in the one or more buildings, the recirculated air comprising internal air in the one or more interior airflow spaces, the internal air including internal air contaminated with one or more of, the one or more biological agents, and the one or more chemical agents.

The at least one compressor assembly further comprises at least one compressor configured to compress, and thereby heat, to an elevated temperature, one or more of, the external air, and the recirculated air, to obtain compressed air, contaminated with one or more of, the one or more biological agents, and the one or more chemical agents. The at least one compressor assembly further comprises at least one plenum coupled to the at least one compressor. The at least one plenum has a plenum inlet with a plenum inlet valve, and has a plenum exit with a plenum exit valve. The at least one plenum is configured to maintain the compressed air at the elevated temperature for an effective dwell time, to purify the compressed air by deactivating one or more of, the one or more biological agents, and the one or more chemical agents, present in the compressed air, to obtain purified air.

The at least one compressor assembly further comprises at least one decompressor coupled to the at least one plenum. The at least one decompressor is configured to decompress the purified air, and thereby cool the purified air to a cooled temperature. The at least one compressor assembly further comprises an air outlet assembly coupled to the at least one decompressor. The air outlet assembly is configured to direct the purified air out of the at least one compressor assembly.

The air purification and management system further comprises an air management system coupled to the air purification system. The air management system further comprises at least one external air intake duct coupled to the air intake assembly of the at least one compressor assembly.

The air management system further comprises an air supply duct assembly coupled between the air outlet assembly of the at least one compressor assembly and the one or more interior airflow spaces, to supply the purified air from the at least one compressor assembly to the one or more interior airflow spaces. The air management system further comprises an air return duct assembly coupled between the one or more interior airflow spaces and the air intake assembly of the at least one compressor assembly, to return the recirculated air from the one or more interior airflow spaces to the at least one compressor assembly.

The air purification and management system further comprises one or more control systems configured to control the air purification system, and configured to control the air management system. The air purification and management system provides a contamination mitigation for occupants of the one or more buildings against contamination by one or more of, the one or more biological agents, and the one or more chemical agents.

In another version of the disclosure, there is provided an air purification system for one or more buildings. The air purification system comprises at least one compressor assembly. The at least one compressor assembly comprises an air intake assembly comprising at least one external air intake opening, and at least two recirculated air intake openings. The air intake assembly is configured to take in one or more of, external air external to the one or more buildings, the external air including external air contaminated with one or more of, one or more biological agents, and one or more chemical agents, and recirculated air from one or more interior airflow spaces in the one or more buildings, the recirculated air comprising internal air in the one or more interior airflow spaces, the internal air including internal air contaminated with one or more of, the one or more biological agents, and the one or more chemical agents.

The at least one compressor assembly further comprises at least one compressor configured to compress, and thereby heat, to an elevated temperature, one or more of, the external air, and the recirculated air, to obtain compressed air, contaminated with one or more of, the one or more biological agents, and the one or more chemical agents. The at least one compressor assembly further comprises at least one plenum coupled to the at least one compressor, the at least one plenum having a plenum inlet with a plenum inlet valve, and having a plenum exit with a plenum exit valve. The at least one plenum is configured to maintain the compressed air at the elevated temperature for an effective dwell time, to purify the compressed air by deactivating one or more of, the one or more biological agents, and the one or more chemical agents, present in the compressed air, to obtain purified air.

The at least one compressor assembly further comprises at least one decompressor coupled to the at least one plenum. The at least one decompressor is configured to decompress the purified air, and thereby cool the purified air to a cooled temperature.

The at least one compressor assembly further comprises an air outlet assembly coupled to the at least one decompressor, and comprising at least one air outlet exhaust opening, and at least one air outlet supply opening. The air outlet assembly is configured to direct the purified air out of the at least one compressor assembly.

The air purification system further comprises a control system configured to control the air purification system. The air purification system is configured for use with an air management system, to supply the purified air to the one or more interior airflow spaces in the one or more buildings, and to receive the recirculated air from the one or more interior airflow spaces, to provide a contamination mitigation for occupants of the one or more buildings against contamination by one or more of, the one or more biological agents, and the one or more chemical agents.

In another version of the disclosure, there is provided a method of purifying and managing air in one or more buildings, to provide a contamination mitigation to occupants of the one or more buildings. The method comprises installing an air purification and management system in the one or more buildings.

The air purification and management system comprises an air purification system. The air purification system comprises at least one compressor assembly. The at least one compressor assembly comprises an air intake assembly configured to take in one or more of, external air external to the one or more buildings, the external air including external air contaminated with one or more of, one or more biological agents, and one or more chemical agents, and recirculated air from one or more interior airflow spaces in the one or more buildings, the recirculated air comprising internal air in the one or more interior airflow spaces, the internal air including internal air contaminated with one or more of, the one or more biological agents, and the one or more chemical agents.

The at least one compressor assembly further comprises at least one compressor. The at least one compressor assembly further comprises at least one plenum coupled to the at least one compressor. The at least one plenum has a plenum inlet with a plenum inlet valve, and has a plenum exit with a plenum exit valve. The at least one compressor assembly further comprises at least one decompressor coupled to the at least one plenum. The at least one compressor assembly further comprises an air outlet assembly coupled to the at least one decompressor. The air outlet assembly is configured to direct purified air out of the at least one compressor assembly.

The air purification and management system further comprises an air management system coupled to the air purification system. The air management system comprises at least one external air intake duct coupled to the air intake assembly of the at least one compressor assembly. The air management system further comprises an air supply duct assembly coupled between the air outlet assembly of the at least one compressor assembly and the one or more interior airflow spaces. The air management system further comprises an air return duct assembly coupled between the one or more interior airflow spaces and the air intake assembly of the at least one compressor assembly. The air purification and management system further comprises one or more control systems configured to control the air purification system, and configured to control the air management system.

The method further comprises intaking, via the air intake assembly of the at least one compressor assembly, one or more of, the external air, and the recirculated air. The method further comprises compressing, and thereby heating, with the at least one compressor, one or more of, the external air, and the recirculated air, to an elevated temperature, to obtain compressed air, contaminated with one or more of, the one or more biological agents, and the one or more chemical agents.

The method further comprises maintaining, with the at least one plenum, the compressed air at the elevated temperature for an effective dwell time, to purify the compressed air by deactivating one or more of, the one or more biological agents, and the one or more chemical agents, present in the compressed air, to obtain purified air. The method further comprises decompressing, with the at least one decompressor, the purified air, to cool the purified air to a cooled temperature.

The method further comprises supplying, with the air supply duct assembly, the purified air from the at least one compressor assembly to the one or more interior airflow spaces. The method further comprises returning, with the air return duct assembly, the recirculated air from the one or more interior airflow spaces to the at least one compressor assembly. The air purification and management system provides the contamination mitigation for the occupants of the one or more buildings against contamination by the one or more biological agents, and the one or more chemical agents.

The features, functions, and advantages that have been discussed can be achieved independently in various versions of the disclosure or may be combined in yet other versions, further details of which can be seen with reference to the following description and drawings.

### BRIEF DESCRIPTION OF THE DRAWINGS

The disclosure can be better understood with reference to the following detailed description taken in conjunction with the accompanying drawings, which illustrate preferred and exemplary versions, but which are not necessarily drawn to scale. The drawings are examples and not meant as limitations on the description or claims.

FIG. 1A is an illustration of a schematic diagram of an exemplary air purification and management system of the disclosure for a building;

FIG. 1B is an illustration of a perspective view of an exemplary air purification and management system of the disclosure for a building;

FIG. 1C is an illustration of a perspective view of the air purification and management system and the building of FIG. 1B, showing external sensors in solid circles in solid lines;

FIG. 1D is an illustration of a perspective view of the air purification and management system and the building of FIG. 1B, showing internal sensors in open circles in solid lines;

FIG. 1E is an illustration of a perspective view of the air purification and management system and the building of FIG. 1B, showing external air intake ducts in solid lines;

FIG. 1F is an illustration of a perspective view of the air purification and management system and the building of FIG. 1B, showing a compressor assembly in solid lines;

FIG. 1G is an illustration of a perspective view of the air purification and management system and the building of FIG. 1B, showing low pressure air supply ducts in solid lines;

FIG. 1H is an illustration of a perspective view of the air purification and management system and the building of FIG. 1B, showing moderate pressure air supply ducts in solid lines;

FIG. 1I is an illustration of a perspective view of the air purification and management system and the building of FIG. 1B, showing low pressure air return ducts in solid lines;

FIG. 1J is an illustration of a perspective view of the air purification and management system and the building of FIG. 1B, showing moderate pressure air return ducts in solid lines;

FIG. 1K is an illustration of a perspective view of the air purification and management system and the building of FIG. 1B, showing an interior airflow space in solid lines located in the building;

FIG. 2A is an illustration of a side view of a version of an air purification system of the disclosure with a compressor assembly, in the form of a reciprocating compressor assembly, in a first cycle position;

FIG. 2B is an illustration of a side view of the air purification system with the compressor assembly, in the form of the reciprocating compressor assembly, of FIG. 2A, in a second cycle position;

FIG. 2C is an illustration of a side view of the air purification system with the compressor assembly, in the form of the reciprocating compressor assembly, of FIG. 2A, in a third cycle position;

FIG. 2D is an illustration of a side view of the air purification system with the compressor assembly, in the form of the reciprocating compressor assembly, of FIG. 2A, in a fourth cycle position;

FIG. 2E is an illustration of a side view of the air purification system with the compressor assembly, in the form of the reciprocating compressor assembly, of FIG. 2A, in a fifth cycle position;

FIG. 2F is an illustration of a side view of the air purification system with the compressor assembly, in the form of the reciprocating compressor assembly, of FIG. 2A, in a sixth cycle position;

FIG. 2G is an illustration of a side view of the air purification system with the compressor assembly, in the form of the reciprocating compressor assembly, of FIG. 2A, in a seventh cycle position;

FIG. 2H is an illustration of a side view of the air purification system with the compressor assembly, in the form of the reciprocating compressor assembly, of FIG. 2A, in an eighth cycle position;

FIG. 3A is an illustration of a side view of a version of an air purification system of the disclosure with a compressor assembly, in the form of a reciprocating compressor assembly, having a plurality of baffles in a plenum;

FIG. 3B is an illustration of a perspective view of a heat exchanger that may be used in a plenum of a compressor assembly;

FIG. 3C is an illustration of a side view of a version of an air purification system of the disclosure with a compressor assembly, in the form of a reciprocating compressor assembly, having a plenum in the form of a coil;

FIG. 4A is an illustration of a graph of temperature versus pressure ratio for a non-ideal compressor at a temperature of sixty-eight degrees Fahrenheit and a compression ratio of 1:1;

FIG. 4B is an illustration of a side view of a non-ideal compressor corresponding to the graph in FIG. 4A, at the temperature of sixty-eight degrees Fahrenheit and the compression ratio of 1:1;

FIG. 4C is an illustration of a graph of temperature versus pressure ratio for a non-ideal compressor at a temperature of four-hundred fifty degrees Fahrenheit and a compression ratio of 3:1;

FIG. 4D is an illustration of a side view of a non-ideal compressor corresponding to the graph in FIG. 4C, at the temperature of four-hundred fifty degrees Fahrenheit and the compression ratio of 3:1;

FIG. 5A is an illustration of a graph of temperature versus pressure ratio for a non-ideal compressor at a temperature of sixty-eight degrees Fahrenheit and a compression ratio of 1:1;

FIG. 5B is an illustration of a side view of a non-ideal compressor corresponding to the graph in FIG. 5A, at the temperature of sixty-eight degrees Fahrenheit and the compression ratio of 1:1;

FIG. 5C is an illustration of a graph of temperature versus pressure ratio for a non-ideal compressor at a temperature of one-thousand one-hundred twenty degrees Fahrenheit and a compression ratio of 10:1;

FIG. 5D is an illustration of a side view of a non-ideal compressor corresponding to the graph in FIG. 5C, at the temperature of one-thousand one-hundred twenty degrees Fahrenheit and the compression ratio of 10:1;

FIG. 6A is an illustration of a graph of temperature versus pressure ratio for a non-ideal compressor at a temperature of sixty-eight degrees Fahrenheit and a compression ratio of 1:1;

FIG. 6B is an illustration of a side view of a non-ideal compressor corresponding to the graph in FIG. 6A, at the temperature of sixty-eight degrees Fahrenheit and the compression ratio of 1:1;

FIG. 6C is an illustration of a graph of temperature versus pressure ratio for a non-ideal compressor at a temperature of two-hundred eighty-seven degrees Fahrenheit and a compression ratio of 2:1;

FIG. 6D is an illustration of a side view of a non-ideal compressor corresponding to the graph in FIG. 6C, at the temperature of two-hundred eighty-seven degrees Fahrenheit and the compression ratio of 2:1;

FIG. 7A is an illustration of a cross-section of a cylinder that may be used in a version of a compressor assembly, the cylinder having a cross-section shape that is a circle shape;

FIG. 7B is an illustration of a cross-section of a prism that may be used in a version of a compressor assembly, the prism having a cross-section shape that is a rectangle shape;

FIG. 7C is an illustration of a cross-section of a prism that may be used in a version of a compressor assembly, the prism having a cross-section shape that is a hexagon shape;

FIG. 7D is an illustration of a cross-section of a tessellation prism arrangement of multiple prisms, each having a cross-section shape that is a rectangle shape;

FIG. 7E is an illustration of a cross-section of a nesting prism arrangement of multiple prisms, each having a cross-section shape that is a hexagon shape;

FIG. 8 is an illustration of a side view of a gap control assembly for a compressor of a version of a compressor assembly;

FIG. 9 is an illustration of a cross-section of a cylinder and a piston, where the piston has a piston ring;

FIG. 10A is an illustration of a cross-section of a piston and a single opening cylinder, where the piston has a piston end surface that matches an interior end surface of the single opening cylinder;

FIG. 10B is an illustration of a side view of the piston and the single opening cylinder of FIG. 10A;

FIG. 11A is an illustration of a cross-section of a piston with a multiple opening cylinder, where the piston has a piston end surface that matches an interior end surface of the multiple opening cylinder;

FIG. 11B is an illustration of a side view of the piston and the multiple opening cylinder of FIG. 11A;

FIG. 12A is an illustration of a side view of a version of an air purification system of the disclosure with a compressor assembly, in the form of a multi-nozzle turbine compressor assembly;

FIG. 12B is an illustration of a side view of a version of an air purification system of the disclosure with a compressor assembly, in the form of a generator turbine compressor assembly;

FIG. 13A is an illustration of a perspective view of a version of an air purification system of the disclosure with a compressor assembly, in the form of a multi-cylinder compressor assembly;

FIG. 13B is an illustration of a perspective view of the multi-cylinder compressor assembly of FIG. 13A, with the plenum removed;

FIG. 14A is an illustration of a perspective view of a duct network of the air management system of the disclosure retrofitted in a retrofitted building;

FIG. 14B is an illustration of a side view of the duct network of FIG. 14A, retrofitted in a retrofitted building, and having an external duct network external to the retrofitted building;

FIG. 15 is an illustration of a flow diagram of an exemplary version of a method of the disclosure; and

FIG. 16 is an illustration of a system block diagram of an exemplary version of an air purification and management system of the disclosure for one or more buildings.

The figures shown in this disclosure represent various aspects of the versions presented, and only differences will be discussed in detail.

### DETAILED DESCRIPTION

Disclosed versions will now be described more fully hereinafter with reference to the accompanying drawings, in which some, but not all of the disclosed versions are shown. Indeed, several different versions may be provided and should not be construed as limited to the versions set forth herein. Rather, these versions are provided so that this disclosure will be thorough and fully convey the scope of the disclosure to those skilled in the art.

This specification includes references to “one version” or “a version”. The instances of the phrases “one version” or “a version” do not necessarily refer to the same version. Particular features, structures, or characteristics may be combined in any suitable manner consistent with this disclosure. All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

As used herein, “comprising” is an open-ended term, and as used in the claims, this term does not foreclose additional structures or steps.

As used herein, “configured to” means various parts or components may be described or claimed as “configured to” perform a task or tasks. In such contexts, “configured to” is used to connote structure by indicating that the parts or components include structure that performs those task or tasks during operation. As such, the parts or components can be said to be configured to perform the task even when the specified part or component is not currently operational (e.g., is not on).

As used herein, the terms “first”, “second”, etc., are used as labels for nouns that they precede, and do not imply any type of ordering (e.g., spatial, temporal, logical, etc.).

As used herein, an element or step recited in the singular and preceded by the word “a” or “an” should be understood as not necessarily excluding the plural of the elements or steps. As used herein, the term “and/or” includes any and all combinations of one or more of the associated listed items. As also used herein, the term “combinations thereof” includes combinations having at least one of the associated listed items, wherein the combination can further include additional, like non-listed items.

As used herein, the phrase “at least one of,” when used with a list of items, means different combinations of one or more of the listed items may be used, and only one of each item in the list may be needed. In other words, “at least one of” means any combination of items and number of items may be used from the list, but not all of the items in the list are required. The item may be a particular object, a thing, or a category.

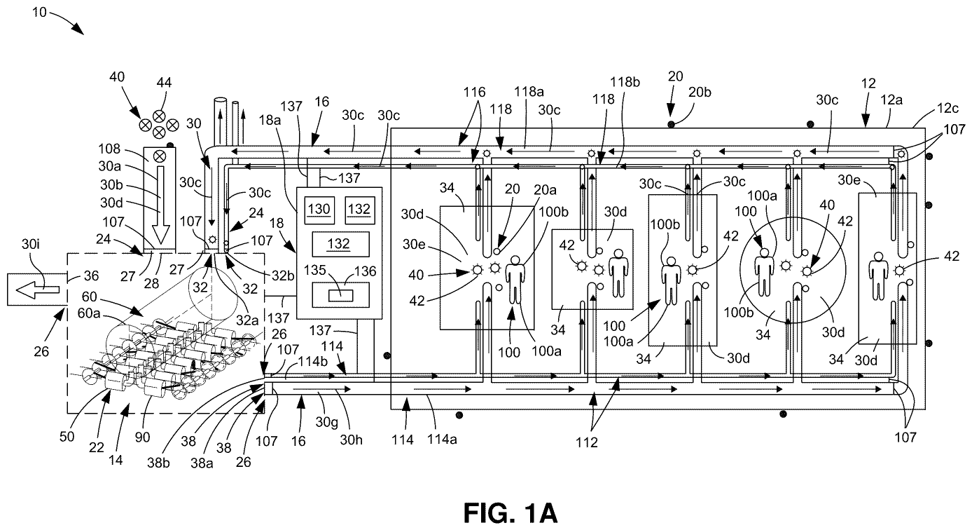

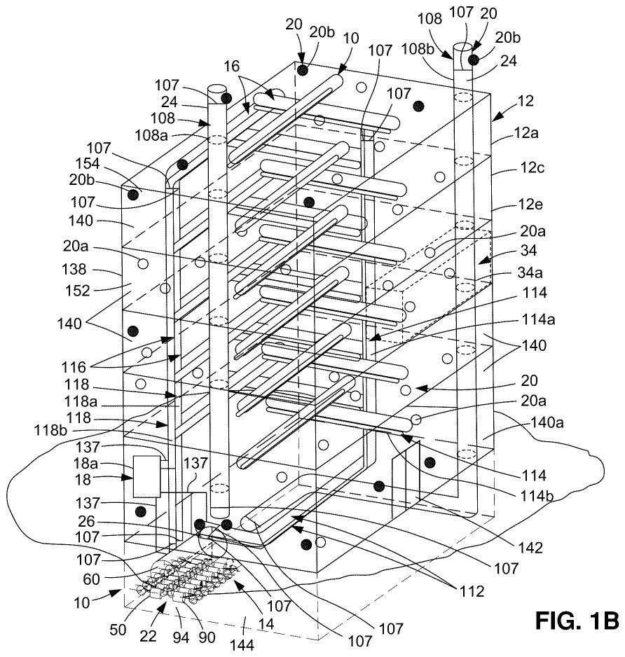

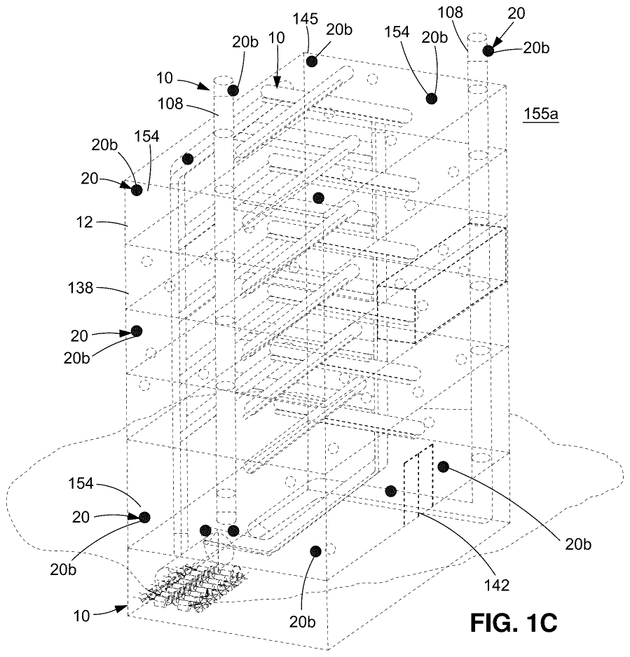

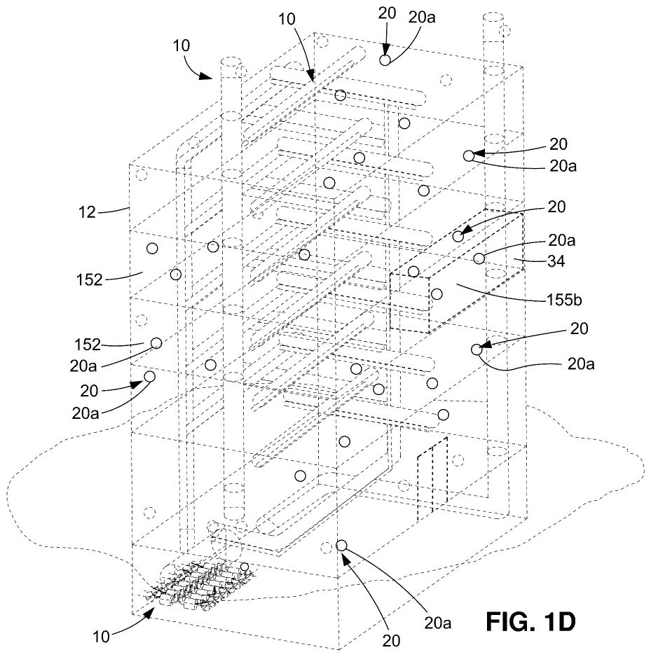

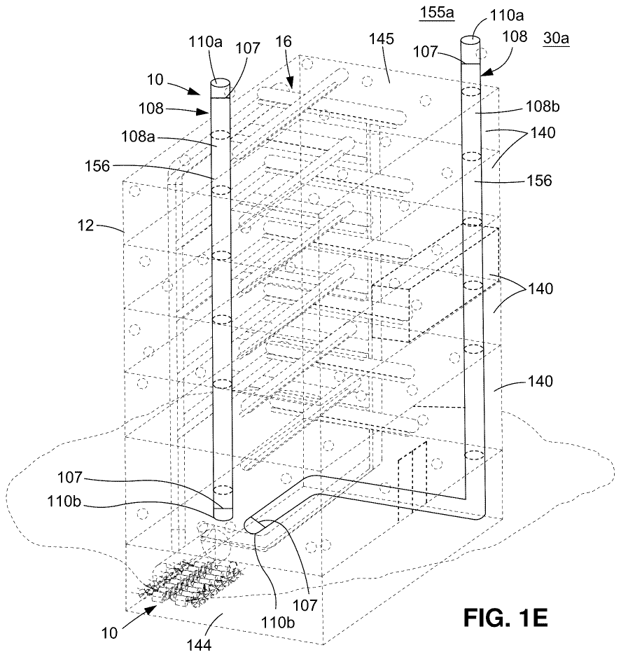

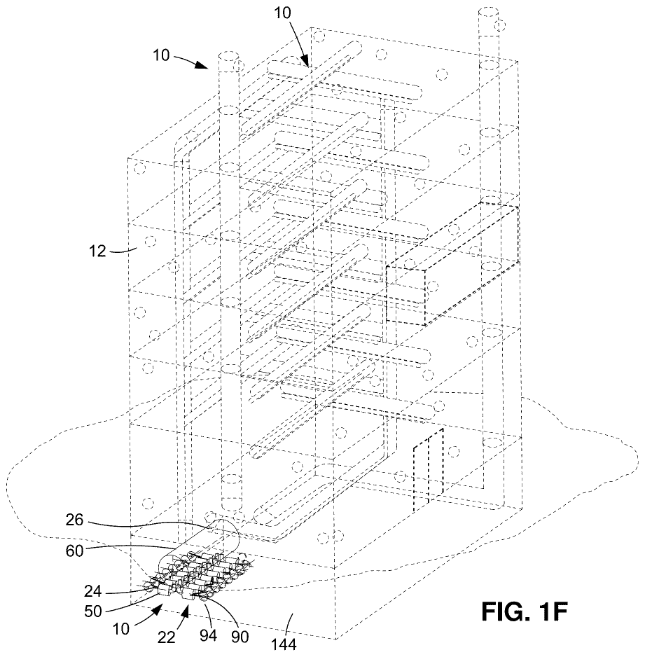

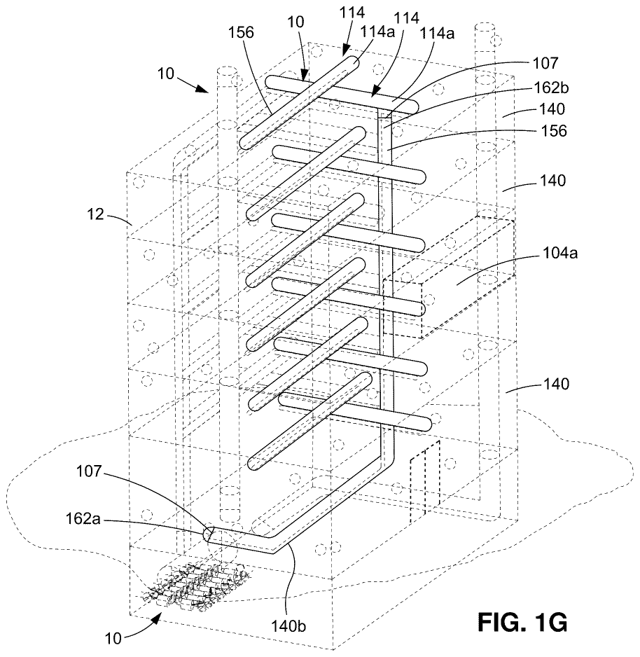

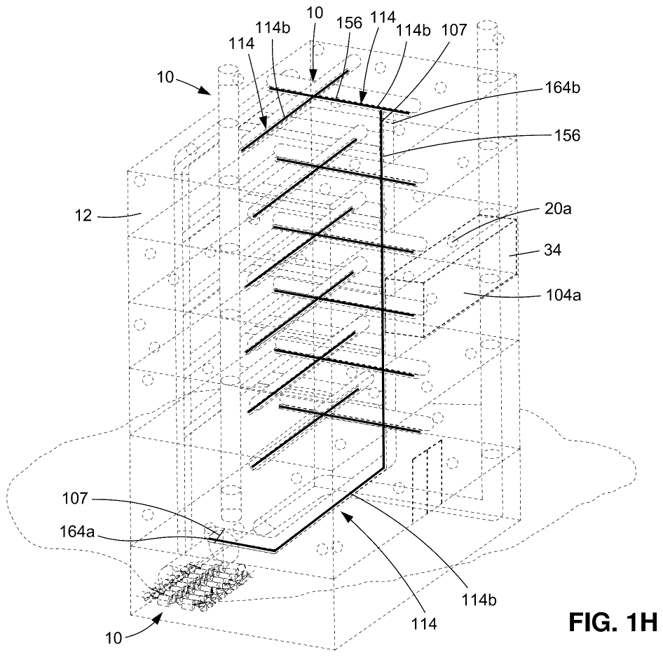

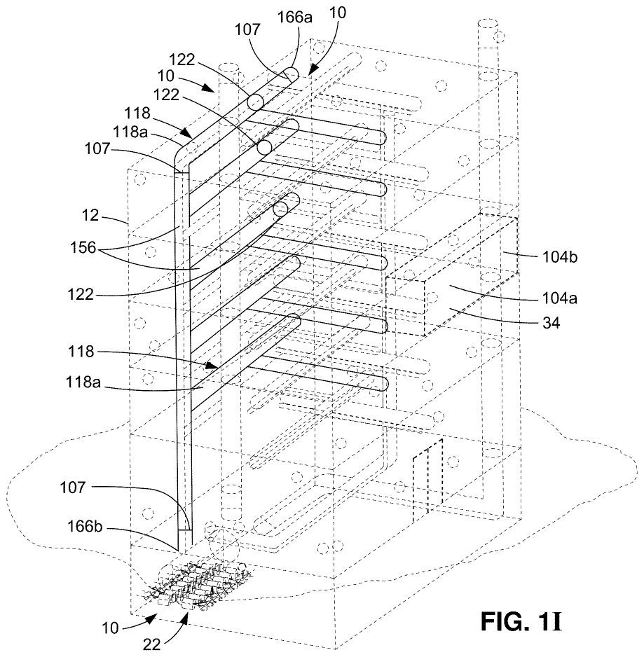

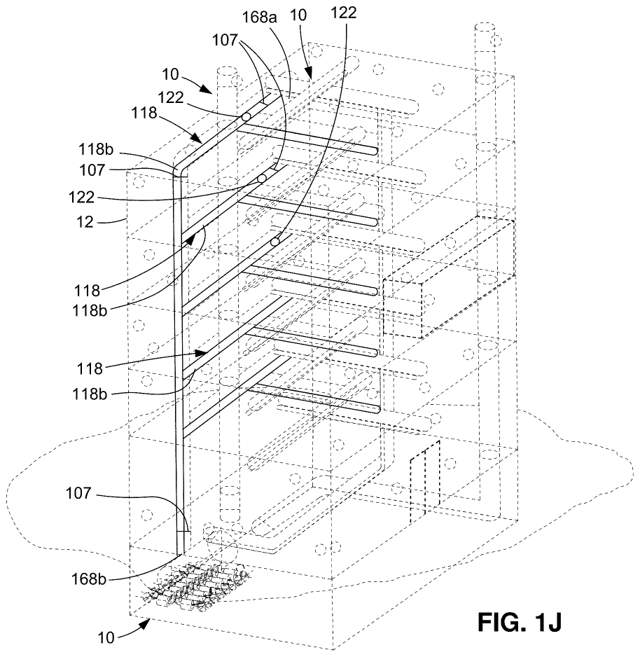

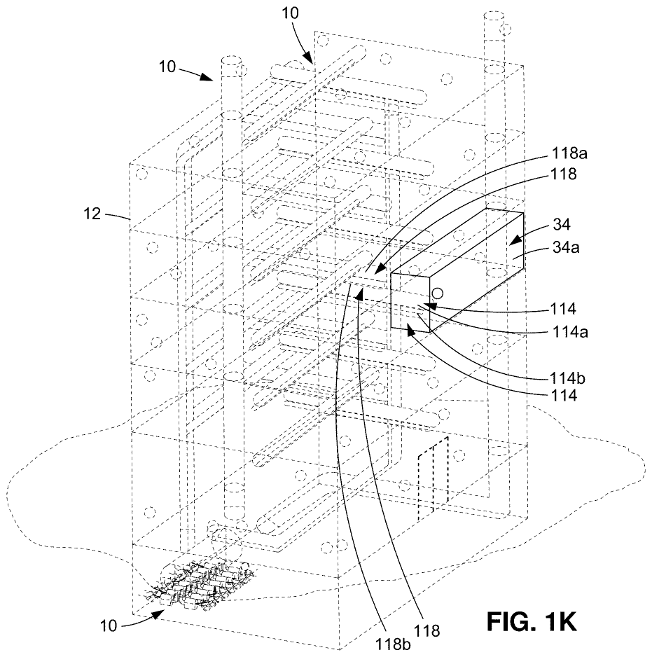

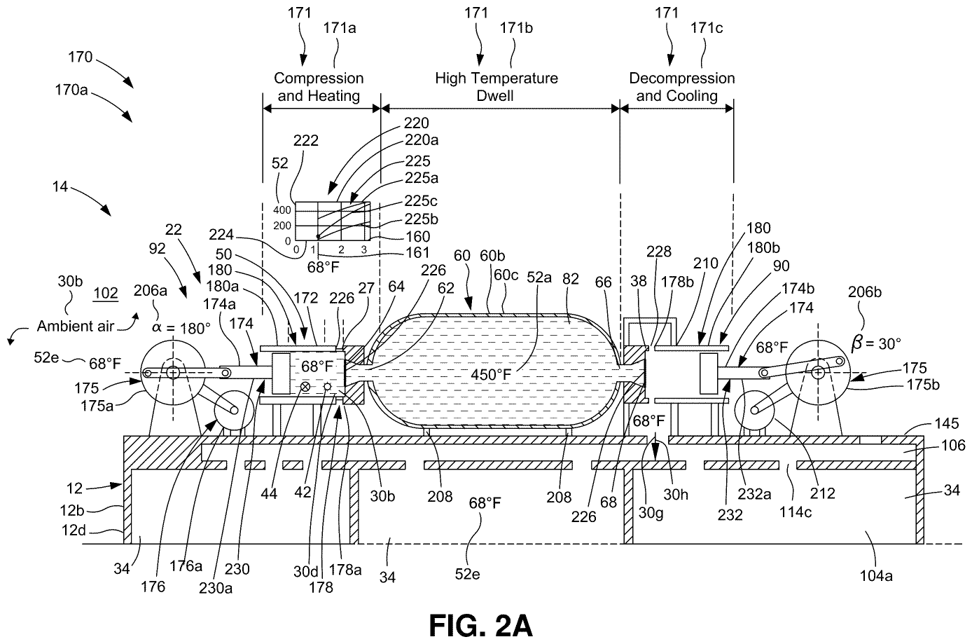

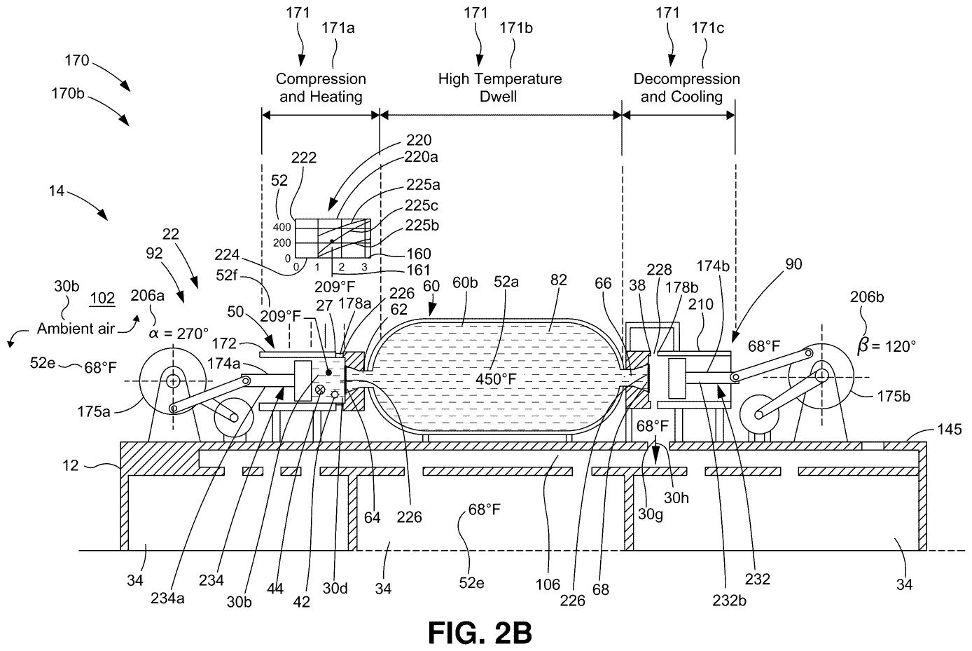

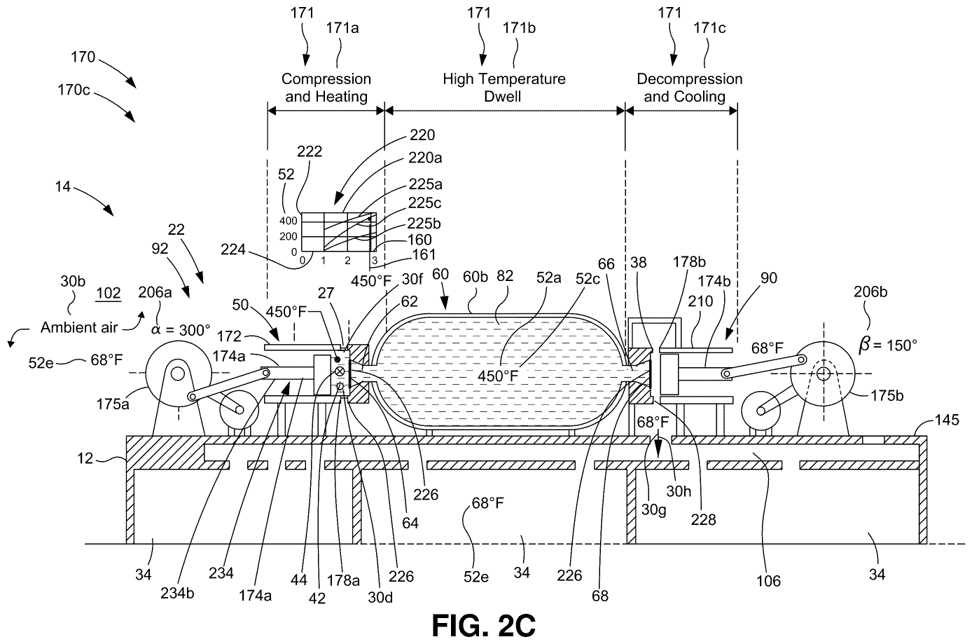

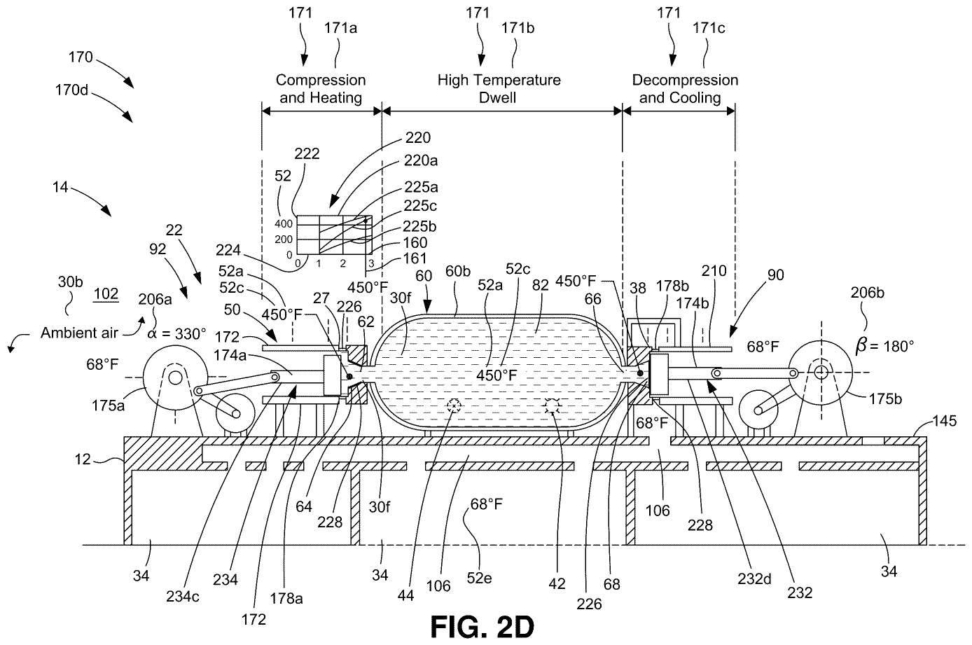

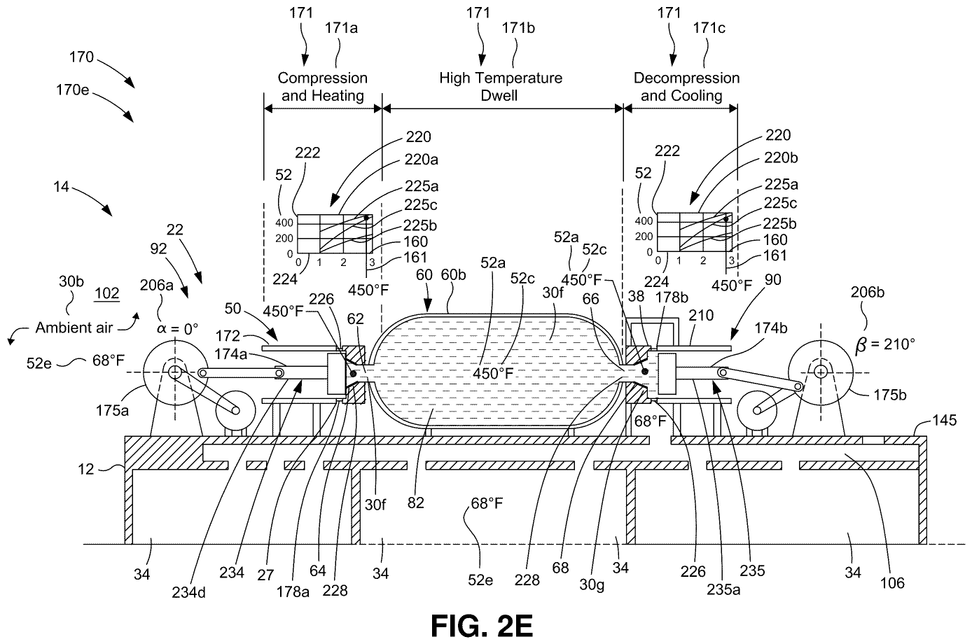

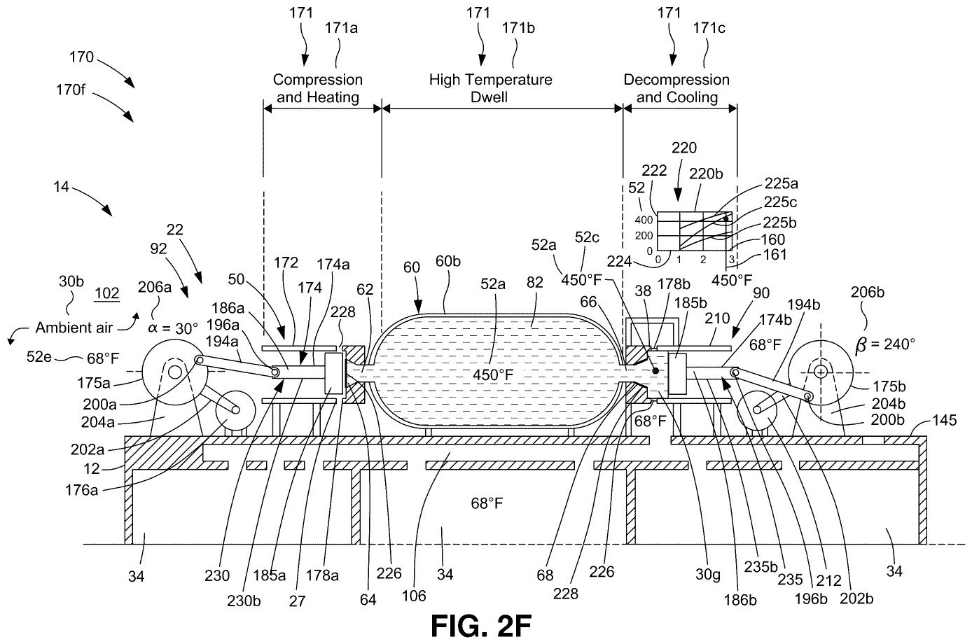

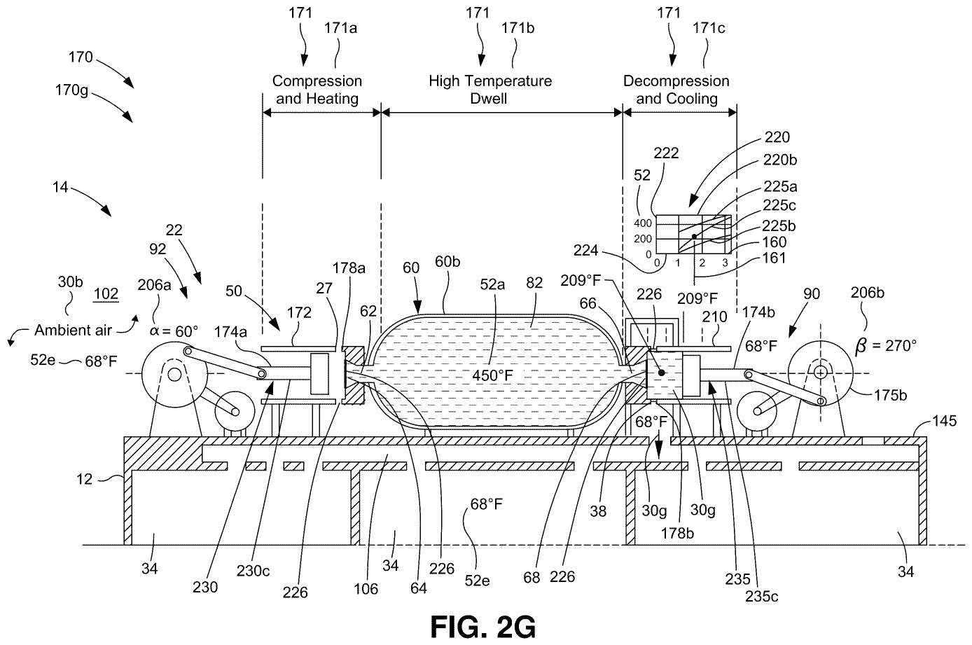

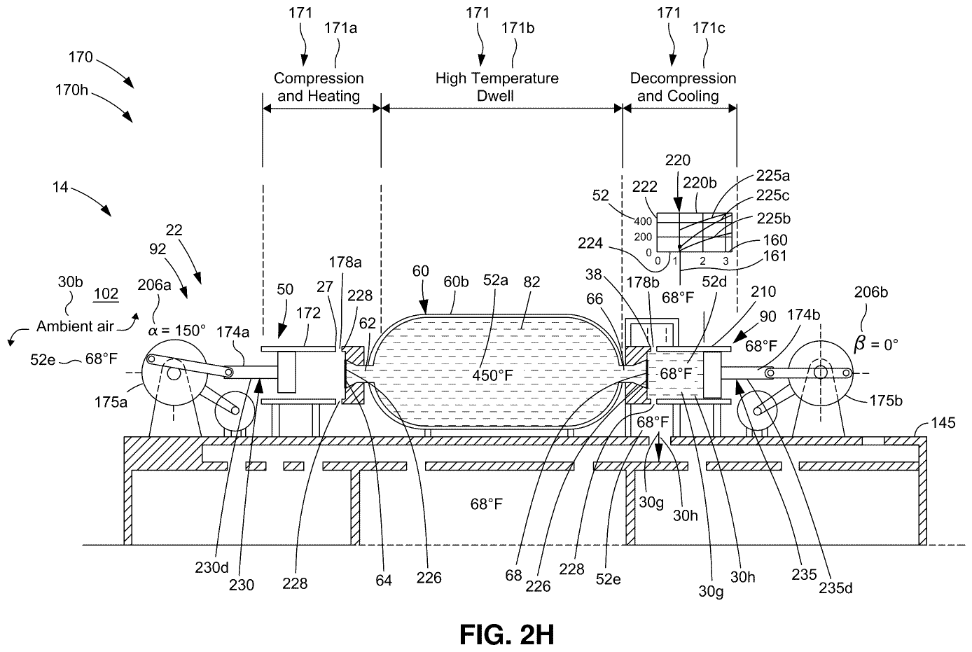

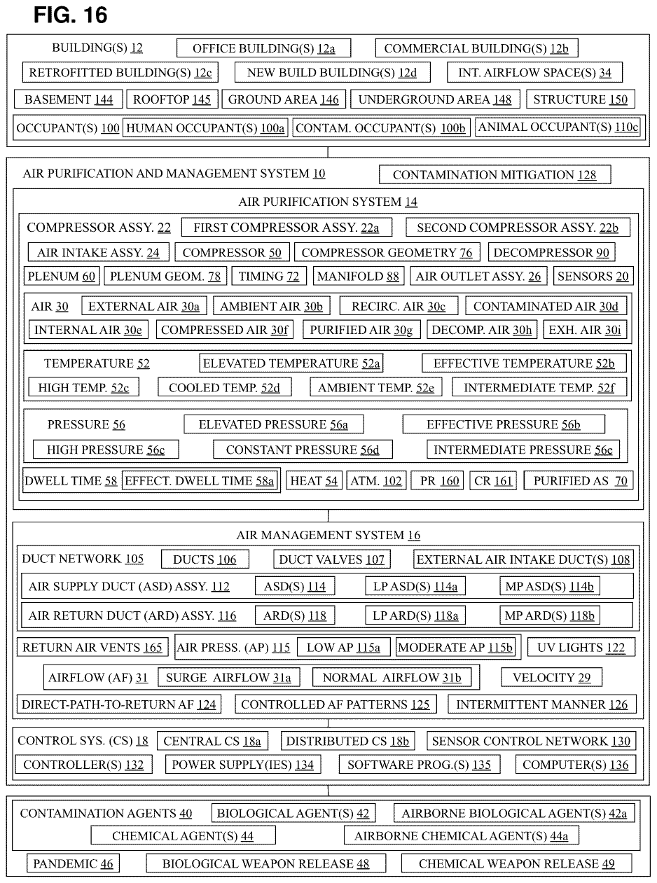

Now referring to FIG. 1A, FIG. 1A is an illustration of a schematic diagram of an exemplary version of an air purification and management system 10 of the disclosure for a building 12, such as an office building 12a. A system block diagram of an exemplary version of the air purification and management system 10 of the disclosure for one or more buildings 12 is also shown in FIG. 16, discussed in further detail below. The air purification and management system 10 is preferably designed for one or more buildings 12, such as one or more office buildings 12a, one or more commercial buildings 12b, or one or more other suitable buildings. The one or more buildings 12, such as the one or more office buildings 12a or one or more commercial buildings 12b, may comprise retrofitted buildings 12c (see FIGS. 1A, 16) that are already existing and already built, and that can be retrofitted with the air purification and management system 10 by installing the air purification and management system 10 at or in the retrofitted building 12c. The one or more buildings 12, such as the one or more office buildings 12a or the one or more commercial buildings 12b, may also comprise new build buildings 12d (see FIGS. 2A, 16) that are newly built, or in the process of being built, and in which the air purification and management system 10 is installed or incorporated at, and/or in, the new build building 12d.

As shown in FIG. 1A, the air purification and management system 10 comprises an air purification system 14, an air management system 16 coupled to the air purification system 14, and one or more control systems 18 configured to control, and controlling, the air purification system 14, and configured to control, and controlling, the air management system 16. The air purification and management system 10 may optionally comprise a plurality of sensors 20 (see FIGS. 1A-1D, 16), such as internal sensors 20a (see FIGS. 1A-1B, 1D) and external sensors 20b (see FIGS. 1A-1C). The sensors 20, such as the internal sensors 20a and the external sensors 20b may comprise temperature sensors, including semiconductor-based sensors, thermocouple sensors, resistance temperature detectors, and negative temperature coefficient thermistors; humidity sensors, including capacitive sensors, resistive sensors, and thermal sensors; pressure sensors; solar insolation sensors; motion sensors to detect occupants 100, such as human occupants 100a, of the building 12; contact sensors to indicate whether a duct or valve is open or closed; sound sensors to indicate sounds, for example, if an occupant 100 in the building 12 sneezes; air quality sensors to monitor toxins, dust, nitrogen dioxide, ozone, and other substances in the air 30 (see FIG. 16); infrared (IR) sensors; sensors that sense or detect biological agents 42 and chemical agents 44, and other suitable sensors 20.

As shown in FIG. 1A, the air purification system 14 comprises at least one compressor assembly 22. As shown in FIG. 1A, the at least one compressor assembly 22 has an air intake assembly 24 and an air outlet assembly 26. As further shown in FIG. 1A, the air intake assembly 24 comprises a plurality of air intake openings 27, including at least one external air intake opening 28, or multiple external air intake openings 28, configured to take in air 30, such as external air 30a, for example, ambient air 30b, external to the one or more buildings 12. As further shown in FIG. 1A, the plurality of air intake openings 27 of the air intake assembly 24 further comprise at least two recirculated air intake openings 32, such as a first recirculated air intake opening 32a and a second recirculated air intake opening 32b, configured to take in air 30, such as recirculated air 30c, from one or more interior airflow spaces 34 in the one or more buildings 12.

The air purification system 14 further comprises an air outlet assembly 26 (see FIGS. 1A, 16) comprising at least one air outlet exhaust opening 36 (see FIG. 1A), or multiple air outlet exhaust openings 36. As shown in FIG. 1A, the air purification system 14 comprises at least two air outlet supply openings 38, such as a first air outlet supply opening 38a, and a second air outlet supply opening 38b.

As shown in FIG. 1A, the external air 30a includes external air 30a in the form of contaminated air 30d contaminated with one or more of, one or more contamination agents 40 (see also FIG. 16), such as one or more biological agents 42 (see also FIG. 16), and one or more chemical agents 44 (see also FIG. 16).

As shown in FIG. 1A, the recirculated air 30c includes recirculated air 30c comprising internal air 30e, from within, or inside, the one or more interior airflow spaces 34. The internal air 30e can include contaminated air 30d contaminated with one or more of, the one or more contamination agents 40 (see also FIG. 16), such as the one or more biological agents 42 (see also FIG. 16), and the one or more chemical agents 44 (see also FIG. 16), breathed by an occupant 100 (see also FIG. 16), such as a human occupant 100a (see also FIG. 16), for example, a contaminated occupant 100b (see also FIG. 16).

As shown in FIG. 16, the biological agents 42 and/or the chemical agents 44 may be in the air 30 as a result of a pandemic 46, for example, a COVID-19 (coronavirus disease 2019) pandemic. As shown in FIG. 16, the biological agents 42 and/or the chemical agents 44 may further be in the air 30, as a result of a biological weapon release 48, or biological weapon (bioweapon) event, or a chemical weapon release 49, or a chemical weapon event, another release event, where one or more biological agents 42, or bioweapons, and/or one or more chemical agents 44, or chemical weapons, are released in the air 30 due to a terrorist event, a war, an attack, an accidental or unintentional release, or another type of release or event.

As shown in FIG. 16, the one or more biological agents 42 may comprise one or more airborne biological agents 42a. The one or more biological agents 42 may further comprise one or more, airborne pathogens; respiratory pathogens; airborne viruses; respiratory viruses; airborne microbes; respiratory microbes; airborne coronaviruses, including SARS-CoV-2 (severe acute respiratory syndrome coronavirus 2), COVID-19 (coronavirus disease 2019), and MERS (Middle East respiratory syndrome); ebola virus; Zika virus; West Nile virus; Marburg virus; influenza virus, including, airborne influenza virus, influenza A virus, influenza B virus, influenza C virus, avian flu, and swine flu; smallpox virus; monkeypox virus;Streptococcus pneumoniae causing pneumonia;Mycobacterium tuberculosis causing tuberculosis; Bacillus anthracis causing anthrax; aspergillus fumigatus causing lung aspergillosis; pneumonic plague; black mold airborne spores; ricin mist; ricin powder; or another type of biological agent 42.

As shown in FIG. 16, the one or more chemical agents 44 may comprise one or more airborne chemical agents 44a. The one or more chemical agents 44 may further comprise one or more, respiratory chemical agents; phenoxy herbicide (Agent Orange); choking agents, such as agents that irritate the nose, throat, and lungs when inhaled, including chlorine, chloropicrin, diphosgene, and phosgene; blister agents, such as agents that affect the eyes, respiratory tract, and skin, including sulfur mustard, nitrogen mustard, lewisite, and phosgene oxime; blood agents, such as agents that inhibit the ability of cells to use oxygen, including hydrogen cyanide, cyanogen chloride, and arsine; and nerve agents, such as agents that block the Acetylcholinesterase (AChE) enzyme in the nervous system, which causes hyper-stimulation of muscles including tabun, sarin, soman, cyclosarin, venomous agent X, or another type of chemical agent 44.

The air intake assembly 24 having the plurality of air intake openings 27 is configured to take in, and does take in, one or more of, the external air 30a external to the one or more buildings 12, where the external air 30a includes ambient air 30b, contaminated air 30d contaminated with one or more of, the one or more biological agents 42, and/or the one or more chemical agents 44, and/or recirculated air 30c from the one or more interior airflow spaces 34 in the one or more buildings 12, where the recirculated air 30c includes internal air 30e in the one or more interior airflow spaces 34, and contaminated air 30d contaminated with one or more of, the one or more biological agents 42, and/or the one or more chemical agents 44, such as breathed by a contaminated occupant 100b (see FIGS. 1A, 16) in an interior airflow space 34.

As shown in FIGS. 1A, 16, the compressor assembly 22 of the air purification system 14 further comprises at least one compressor 50. The compressor 50 is configured to compress, and compresses, and thereby heats, one or more of, the external air 30a, and the recirculated air 30c, to obtain compressed air 30f, such as compressed air 30f contaminated with one or more of, the one or more biological agents 42, and the one or more chemical agents 44. The compressor 50 compresses, and thereby heats, one or more of, the external air 30a, and the recirculated air 30c, to and at a temperature 52 (see FIG. 16), such as an elevated temperature 52a (see FIG. 16), and to and at an effective temperature 52b (see FIG. 16), such as a high temperature 52c (see FIG. 16), and to and at a pressure 56 (see FIG. 16), such as an elevated pressure 56a (see FIG. 16), and an effective pressure 56b (see FIG. 16), such as a high pressure 56c (see FIG. 16).

One or more of, the external air 30a, and/or the recirculated air 30c, are preferably compressed, and thereby heated, to and at the temperature 52, such as the elevated temperature 52a, in a temperature range of from 140 °F (one-hundred forty degrees Fahrenheit) (60 °C (sixty degrees Celsius)) to 1500 °F (one thousand five hundred degrees Fahrenheit) (816 °C (eight hundred sixteen degrees Celsius)), and more preferably, in a temperature range of from 158 °F (one hundred fifty-eight degrees Fahrenheit) (70 °C (seventy degrees Celsius)) to 600 °F (six hundred degrees Fahrenheit) (316 °C (three-hundred sixteen degrees Celsius)).

As shown in FIGS. 1A, 16, the compressor assembly 22 of the air purification system 14 further comprises at least one plenum 60 coupled to the at least one compressor 50. The at least one plenum 60 has a plenum inlet 62 (see FIG. 2A) with a plenum inlet valve 64 (see FIG. 2A), and has a plenum exit 66 (see FIG. 2A) with a plenum exit valve 68 (see FIG. 2A). As shown in FIG. 1A, the plenum 60 is in the form of a plenum tank 60a. The at least one plenum 60 is configured to maintain, and maintains, the compressed air 30f, that includes one or more of, the external air 30a and the recirculated air 30c, at the elevated temperature 52a for a dwell time 58 (see FIG. 16), such as an effective dwell time 58a (see FIG. 16), to purify the compressed air 30f, by deactivating or destroying one or more of, the one or more biological agents 42, and the one or more chemical agents 44, present in the compressed air 30f, to obtain purified air 30g. The plenum 60 is configured to purify, and does purify, the compressed air 30f to decontaminate the compressed air 30f. The plenum 60 of the compressor assembly 22 is further configured to repurify, and repurifies, the recirculated air 30c.

The compressed air 30f, that includes one or more of, the external air 30a, and/or the recirculated air 30c, is preferably maintained at a heat 54 (see FIG. 16) at the temperature 52, such as the elevated temperature 52a, in a temperature range of from 140 °F (one-hundred forty degrees Fahrenheit) (60 °C (sixty degrees Celsius)) to 1500 °F (one thousand five hundred degrees Fahrenheit) (816 °C (eight hundred sixteen degrees Celsius)), and more preferably, in a temperature range of from 158 °F (one hundred fifty-eight degrees Fahrenheit) (70 °C (seventy degrees Celsius)) to 600 °F (six hundred degrees Fahrenheit) (316 °C (three-hundred sixteen degrees Celsius)).

The compressed air 30f, that includes one or more of, the external air 30a, and/or the recirculated air 30c, is preferably maintained at the temperature 52, such as the elevated temperature 52a, for the dwell time 58, such as the effective dwell time 58a, comprising a range of 0.5 (zero point five) second to 30 (thirty) minutes, and more preferably, heated at the elevated temperature 52a for the effective dwell time 58a comprising a range of 1 (one) second to 5 (five) minutes.

A timing 72 (see FIG. 16) of the plenum inlet valve 64 opening is adjustable, to regulate the elevated temperature 52a and the elevated pressure 56a (see FIG. 16) inside the plenum 60. A compression ratio (CR) 161 (see FIG. 16) can also be adjusted with the timing 72 (see FIG. 16) of the opening of the plenum inlet valve 64. A compressor geometry 76 (see FIG. 16) of the compressor 50 and a plenum geometry 78 (see FIG. 16) of the plenum 60 are fixed.

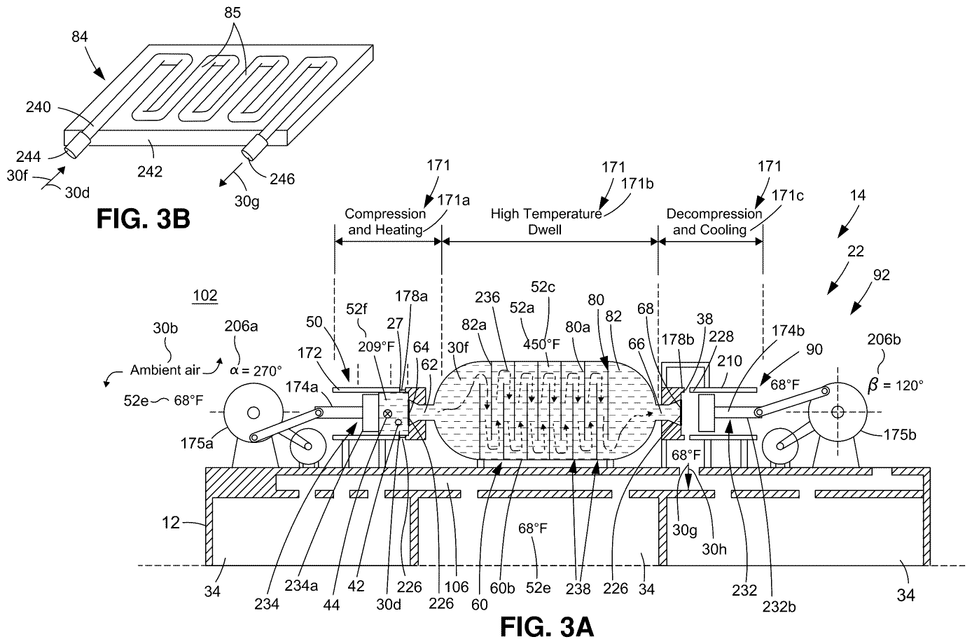

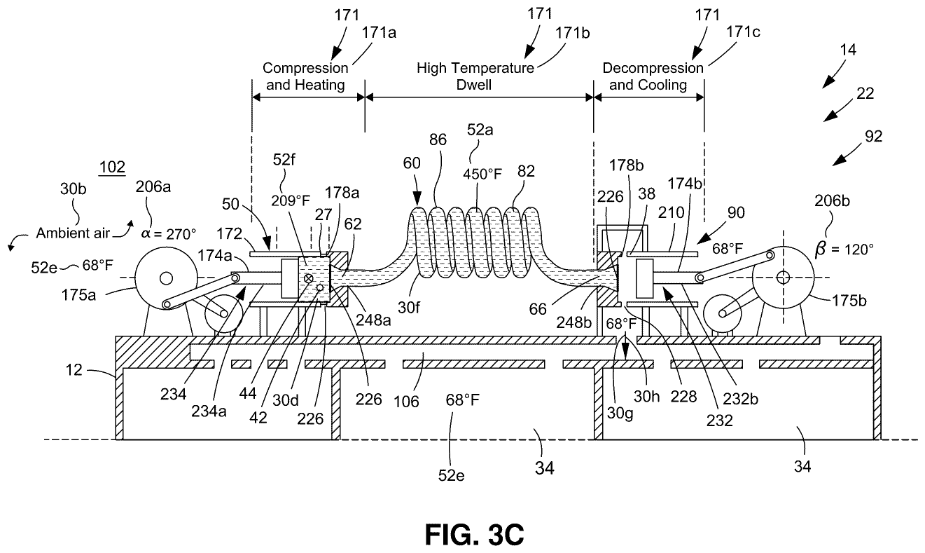

In one version, the plenum 60 comprises a plurality of baffles 80 (see FIG. 3A) positioned within an interior 82 (see FIG. 3A) of the plenum 60. The baffles 80 are discussed in further detail below with regard to FIG. 3A. In another version, the plenum 60 comprises a heat exchanger 84 (see FIG. 3B) with internal airflow passages 85 (see FIG. 3B) for heating or cooling the compressed air 30f, that includes one or more of, the external air 30a, and/or the recirculated air 30c, within the at least one plenum 60. The heat exchanger 84 is discussed in further detail below with regard to FIG. 3B. In yet another version, the plenum 60 comprises a coil 86 (see FIG. 3C), discussed in further detail below with regard to FIG. 3C.

The plenum 60 may further comprise a manifold 88 (see FIG. 16). The plenum 60, such as a plenum tank 60a, may further comprise heat exchangers in the interior 82 to heat or cool the air 30, such as the compressed air 30f.

As shown in FIGS. 1A, 16, the compressor assembly 22 of the air purification system 14 further comprises at least one decompressor 90 coupled to the at least one plenum 60. The at least one decompressor 90 is configured to decompress, and decompresses, and thereby cools, the purified air 30g to a cooled temperature 52d (see FIG. 16), such as a cooled temperature 52d preferably in a range of from 25 °F (twenty-five degrees Fahrenheit) to 80 °F (eighty degrees Fahrenheit), and more preferably, in a range of from 65 °F (sixty-five degrees Fahrenheit) to 74°F. (seventy-four degrees Fahrenheit), to obtain decompressed air 30h. The cooled temperature 52d is less than, or lower than, the elevated temperature 52a.

The air outlet assembly 26, such as the air outlet supply openings 38, are coupled to the at least one decompressor 90. The air outlet assembly 26, such as the air outlet supply openings 38, is/are configured to direct the purified air 30g out of the at least one compressor assembly 22, such as out of the decompressor 90. The air outlet assembly 26 may comprise at least one air outlet supply opening 38, and preferably two air outlet supply openings 38, such as the first air outlet supply opening 38a (see FIG. 1A) and the second air outlet supply opening 38b (see FIG. 1A). The air outlet assembly 26 may further comprise at least one air outlet exhaust opening 36 (see FIG. 1A) to exhaust or output exhaust (EXH.) air 30i (see FIGS. 1A, 16). The exhaust air 30i may include purified air 30g and/or contaminated air 30d.

The at least one compressor assembly 22 is configured to supply, and supplies, all or some of the purified air 30g (see FIGS. 1A, 16), to the air management system 16, and the compressor assembly 22 provides a purified air supply 70 (see FIG. 16), or a clean air supply, to the air management system 16. The exhaust air 30i may comprise some purified air 30g exhausted out of the at least one air outlet exhaust opening 36 of the at least one compressor assembly 22, and exhausted into the atmosphere (ATM.) 102 (see FIGS. 1A, 16) external to the one or more buildings 12.

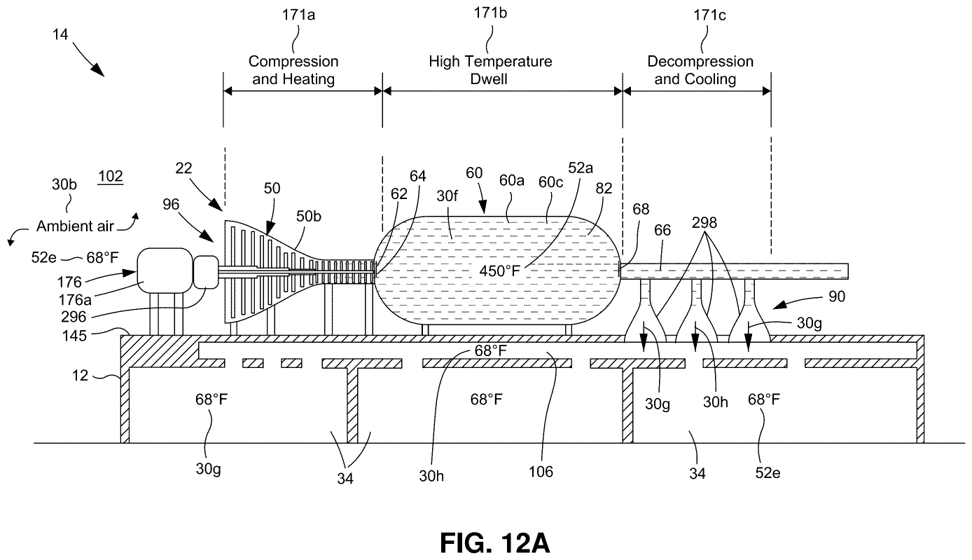

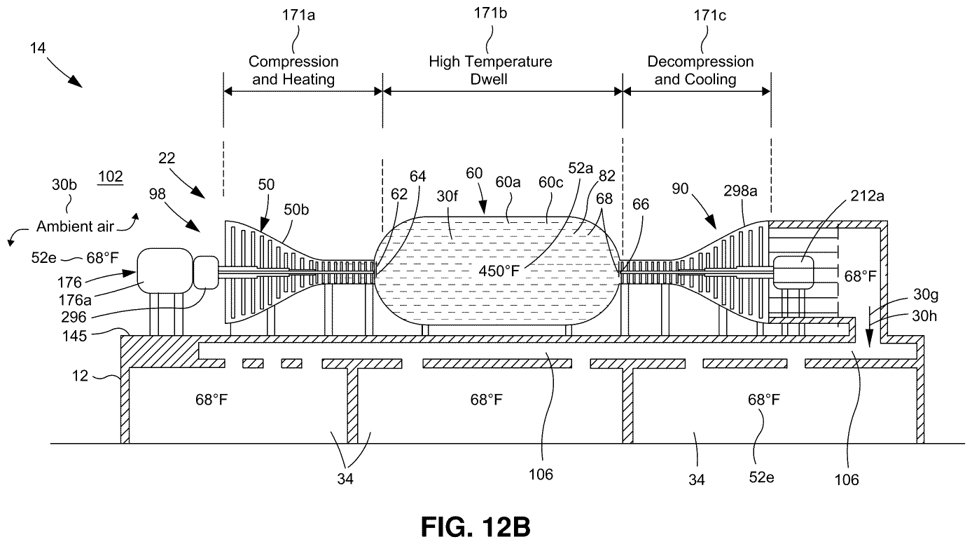

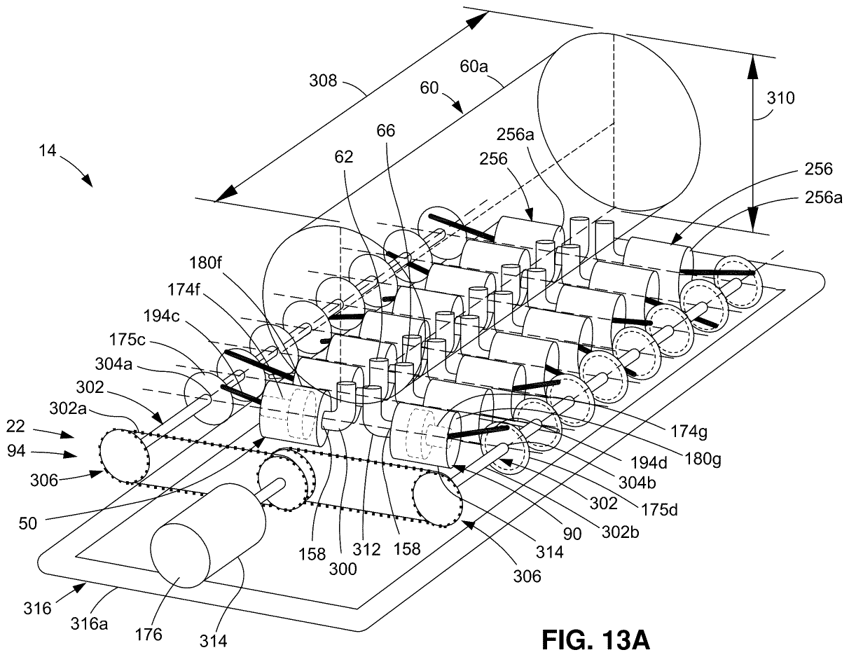

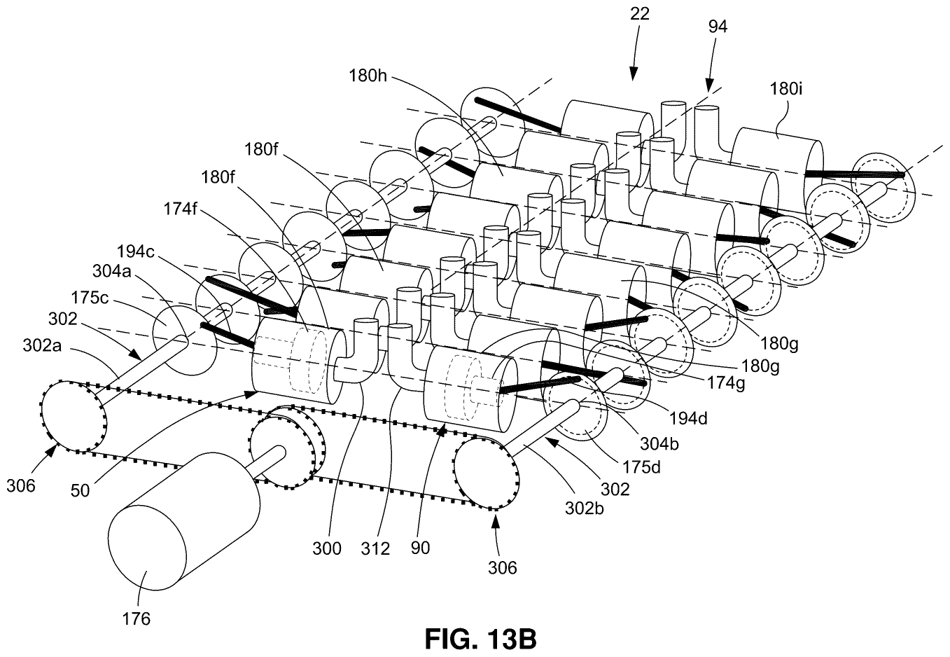

In one version, the compressor assembly 22 comprises a reciprocating compressor assembly 92 (see FIGS. 2A-2H), discussed in further detail below with regard to FIGS. 2A-2H. In another version, the compressor assembly 22 comprises a multi-cylinder compressor assembly 94 (see FIGS. 1A, 13A), discussed in further detail below with regard to FIG. 13A. In yet another version, the compressor assembly 22 comprises a multi-nozzle turbine compressor assembly 96 (see FIG. 12A), discussed in further detail below with regard to FIG. 12A. In yet another version, the compressor assembly 22 comprises a generator turbine compressor assembly 98 (see FIG. 12B), discussed in further detail below with regard to FIG. 12B.

The compressor assembly 22 of the air purification system 14 of the air purification and management system 10 preferably supplies the purified air 30g having a greatly increased reliability, for example, a 10⁻⁹ reliability, that occupants 100 (see FIGS. 1A, 16), such as human occupants 100a (see FIG. 1A), and/or animal occupants 100c (see FIG. 16), such as dogs and cats or other types of animals, are not contaminated by one or more of, the one or more biological agents 42, and the one or more chemical agents 44, released in external air 30a, such as ambient air 30b, to become contaminated air 30d external to the one or more buildings 12, or outside the one or more buildings 12 in atmosphere (ATM.) 102 (see FIG. 16), and/or released by one or more contaminated occupants 100b (see FIG. 1A) located inside a building interior 104a (see FIG. 1A) of the building 12, such as in an interior airflow space interior 104b (see FIG. 1A) of the interior airflow spaces 34 inside the building 12.

The purified air 30g comprising the purified air supply 70 (see FIG. 16), or the clean air supply, is supplied, or flows, out the at least two air outlet supply openings 38 of the at least one compressor assembly 22, and into the air management system 16. The air management system 16 is coupled to the air purification system 14, and comprises a duct network 105 (see FIGS. 1A, 1B, 16) comprised of a plurality of ducts 106 (see FIGS. 1A, 1B, 16) that may have one or more duct valves 107 (see FIGS. 1A, 16). As shown in FIG. 1A, the air management system 16 comprises at least one external air intake duct 108 coupled to the at least one external air intake opening 28 of the at least one compressor assembly 22. As shown in FIGS. 1B, 1E, the air management system 16, in one version, has two external air intake ducts 108, including a first external air intake duct 108a, and a second external air intake duct 108b. Each external air intake duct 108 has a first end opening 110a (see FIG. 1E) in contact with the external air 30a, or ambient air 30b, external to the building 12, and each external air intake duct 108 has a second end opening 110b (see FIG. 1E) coupled to the external air intake opening 28 of the air intake assembly 24, for example, a first external air intake opening 28a, and a second external air intake opening 28b, of the compressor assembly 22.

As shown in FIG. 1A, the air management system 16 further comprises an air supply duct assembly 112 coupled between the air outlet assembly 26 of the at least one compressor assembly 22 and the one or more interior airflow spaces 34, to supply the purified air 30g from the at least one compressor assembly 22 to the one or more interior airflow spaces 34. In one version, as shown in FIG. 1A, the air supply duct assembly 112 comprises a plurality of air supply ducts 114, including a plurality of low pressure air supply ducts 114a, and a plurality of moderate pressure air supply ducts 114b. Each of the plurality of air supply ducts 114 has an air pressure (PRESS.) (AP) 115 (see FIG. 16) inside each air supply duct 114. Preferably, in one version, each of the plurality of low pressure air supply ducts 114a has a low air pressure (AP) 115a (see FIG. 16) in a range of from 0.002 psi (point zero two pound per square inch) to 0.05 psi (point zero five pound per square inch). In other versions, the low air pressure 115a may be less than 0.002 psi or greater than 0.05 psi.

Preferably, in one version, each of the plurality of moderate pressure air supply ducts 114b has a moderate air pressure (AP) 115b (see FIG. 16) in a range of from 0.05 psi (point zero five pound per square inch) to 5.0 psi (five pounds per square inch). In other versions, the moderate air pressure 115b may be less than 0.05 psi or greater than 5 psi.

As shown in FIG. 1A, the air management system 16 further comprises an air return duct assembly 116 coupled between the one or more interior airflow spaces 34 and the air intake assembly 24 of the at least one compressor assembly 22, to return the recirculated air 30c from the one or more interior airflow spaces 34 to the at least one compressor assembly 22 for purification or repurification. The recirculated air 30c comprises the internal air 30e in the one or more interior airflow spaces 34 mixed with the purified air 30g from the compressor assembly 22. The internal air 30e includes, or may include, internal air 30e contaminated with one or more of, the one or more biological agents 42, and the one or more chemical agents 44, breathed by a contaminated occupant 100b in the building 12.

In one version, as shown in FIG. 1A, the air return duct assembly 116 comprises a plurality of air return ducts 118, including a plurality of low pressure air return ducts 118a, and a plurality of moderate pressure air return ducts 118b. Each of the plurality of air return ducts 118 has an air pressure 115 (see FIG. 16) inside each air return duct 118. Preferably, in one version, each of the plurality of low pressure air return ducts 118a has a low air pressure 115a in a range of from 0.002 psi (point zero zero two pound per square inch) to 0.05 psi (point zero five pound per square inch). In other versions, the low air pressure 115a may be less than 0.002 psi or greater than 0.05 psi.

Preferably, in one version, each of the plurality of moderate pressure air return ducts 118b has a moderate air pressure 115b in a range of from 0.05 psi (point zero five pound per square inch) to 5.0 psi (five pounds per square inch). In other versions, the moderate air pressure 115b may be less than 0.05 psi or greater than 5.0 psi.

In one version, an interior 120a (see FIG. 1I) of one or more of the plurality of low pressure air return ducts 118a (see FIG. 1I), and an interior 120b (see FIG. 1J) of one or more of the plurality of moderate pressure air return ducts 118b (see FIG. 1I), contain one or more ultraviolet (UV) lights 122 (see FIGS. 1I, 1J, 16). The one or more ultraviolet lights 122 may be optionally used and located in the air return ducts 118 (see FIGS. 1I, 1J) to aid in deactivating or destroying one or more of the one or more biological agents 42 and/or one or more of the chemical agents 44. As an alternative to the ultraviolet lights 122, or in addition to the ultraviolet lights 122, the air return ducts 118 may have one or more grates with heated wires inside the air return ducts 118, or another suitable device or assembly to aid in deactivating or destroying one or more of the one or more biological agents 42 and/or one or more of the chemical agents 44.

The one or more interior airflow spaces 34 are designed for a direct-path-to-return airflow 124 (see FIG. 16). The direct-path-to-return airflow 124 is an airflow field designed and achieved for which there is a very low probability of an occupant 100, such as a human occupant 100a, of the building 12, breathing air 30, such as contaminated air 30d, that has already been breathed by another occupant 100, such as another human occupant 100a including a contaminated occupant 100b, nearby in the interior airflow space 34, and in turn, any other occupant 100, such as any other human occupant 100a, including a contaminated occupant 100b, in the building 12 in any other interior airflow space 34.

The air supply duct assembly 112 and the air return duct assembly 116 create controlled airflow (AF) patterns 125 (see FIG. 16) in the one or more interior airflow spaces 34 in an intermittent manner 126 (see FIG. 16), to mitigate the occupants 100 in the building 12 from breathing contaminated air 30d contaminated by one or more of, the one or more biological agents 42, and the one or more chemical agents 44. The intermittent manner 126 means the purified air 30g is introduced, or flows, into the interior airflow spaces 34 only when the occupant 100, such as the human occupant 100a, is present in the interior airflow space 34. For example, airflow 31 (see FIG. 16) is preferably regulated and turned on and off, and a velocity 29 (see FIG. 16) of the airflow 31 is preferably adjusted to follow or track the occupant 100, as the occupant 100 moves, or is stationary, for a short period of time, such as walking in an aisleway, or briefly stopping in the aisleway and then resuming walking in the aisleway of the building 12.

The one or more interior airflow spaces 34 in the one or more buildings 12, such as the one or more office buildings 12a, may comprise conference rooms 34a (see FIGS. 1B, 1K) cubicles, office cubicles, offices, aisleways, restrooms, auditoriums, elevators, cafeterias, lunch rooms, or other suitable interior airflow spaces 34. The air purification and management system 10 provides a contamination mitigation 128 (see FIG. 16) for occupants 100 (see FIGS. 1A, 16), such as human occupants 100a (see FIGS. 1A, 16), in the one or more buildings 12 against contamination by one or more of, the one or more biological agents 42, and the one or more chemical agents 44.

As shown in FIG. 1A, in one version, the control system 18 comprises a sensor control network 130 with one or more controllers 132, one or more power supplies 134 to power the control system 18, one or more software programs 135, and one or more computers 136 configured to run the one or more software programs 135. As shown in FIG. 1A, in one version, the control system 18 is a central control system 18a (see also FIG. 16), coupled to the compressor assembly 22, the air supply duct assembly 112, and the air return duct assembly 116, via connector elements 137, such as wired connectors or wireless connectors. In another version, the control system 18 comprises a distributed control system 18b, in which the sensor control network 130, controllers 132, power supplies 134, and computers 136 with software programs 135, are distributed at each interior airflow space 34 and/or secondary interior location 152, or at several interior airflow spaces 34 and/or secondary interior locations 152 on the same floor 140 (see FIG. 1B) or on adjacent floors 140.

Now referring to FIGS. 1B-1K, FIGS. 1B-1K show a perspective view of an exemplary air purification and management system 10 of the disclosure for a building 12, such as an office building 12a, for example, a retrofitted building 12c. FIG. 1B is an illustration of a perspective view of the exemplary air purification and management system 10 for the building 12, such as the office building 12a, showing all the features of the air purification and management system 10. FIGS. 1C-1K show individual features of the air purification and management system 10, with the individual features shown in solid lines, and the other features shown in dotted lines.

As shown in FIG. 1B, the air purification and management system 10 is located in, and at, the building 12, such as an office building 12a, for example, a five-story building 12e. As shown in FIG. 1B, the building 12, such as the five-story building 12e, has a building skin 138, five (5) floors 140, including a ground floor 140a, a building entrance 142 on the ground floor 140a, and a basement 144.

As shown in FIG. 1B, in one version, the compressor assembly 22 is located in the basement 144 (see also FIG. 16) of the building 12, such as the office building 12a, below the ground floor 140a. In other versions, the compressor assembly 22 may be located on a rooftop 145 (see FIGS. 2A, 16) of one or more of the one or more buildings 12, may be located on a ground area 146 (see FIG. 16) adjacent to one or more of the one or more buildings 12, may be located in an underground area 148 (see FIG. 16) adjacent to one or more of the one or more buildings 12, may be located in a structure 150 (see FIG. 16), such as a utility building or a utility shed, a storage building or storage shed, or another suitable structure, in proximity to one or more of the one or more buildings 12.

As shown in FIG. 1B, the air purification and management system 10 comprises, as discussed above, the air purification system 14 with the compressor assembly 22. As shown in FIG. 1B, in this version, the compressor assembly 22 is in the form of the multi-cylinder compressor assembly 94. In other versions, the compressor assembly 22 may be in the form of the reciprocating compressor assembly 92 (see FIG. 2A), the multi-nozzle turbine compressor assembly 96 (see FIG. 12A), the generator turbine compressor assembly 98 (see FIG. 12B), or another suitable compressor assembly 22. As shown in FIG. 1B, the compressor assembly 22, such as the multi-cylinder compressor assembly 94, comprises the air intake assembly 24, the compressor 50 coupled to the air intake assembly 24, the plenum 60 coupled to the compressor 50, the decompressor 90 coupled to the plenum 60, and the air outlet assembly 26 coupled to the decompressor 90. The compressor assembly 22 supplies the purified air supply 70 (see FIG. 16) of purified air 30g (see FIGS. 1A, 16) to the air management system 16 (see FIG. 1B). The air outlet assembly 26 directs the purified air 30g (see FIG. 1A) out of the compressor assembly 22 and into the air management system 16.

As further shown in FIG. 1B, the air purification and management system 10 comprises the air management system 16 coupled to the air purification system 14. The air management system 16 comprises external air intake ducts 108, such as the first external air intake duct 108a and the second external air intake duct 108b, coupled to the air intake assembly 24 of the compressor assembly 22.

As shown in FIG. 1B, the air management system 16 further comprises the air supply duct assembly 112 coupled between the air outlet assembly 26 of the compressor assembly 22 and an exemplary interior airflow space 34, to supply the purified air 30g (see FIGS. 1A, 16) from the compressor assembly 22 to the interior airflow space 34. As shown in FIG. 1B, the air supply duct assembly 112 comprises the plurality of air supply ducts 114, including the low pressure air supply ducts 114a and the moderate pressure air supply ducts 114b.

As shown in FIG. 1B, the air management system 16 further comprises the air return duct assembly 116 coupled between the exemplary interior airflow space 34 and the air intake assembly 24 of the compressor assembly 22, to return the recirculated air 30c (see FIGS. 1A, 16) from the interior airflow space 34 to the compressor assembly 22. As shown in FIG. 1B, the air return duct assembly 116 comprises the plurality of air return ducts 118, including the low pressure air return duct 118a and the moderate pressure air return duct 118b. The interior airflow space 34 is designed for the direct-path-to-return airflow 124 (see FIG. 16). In another version, the air management system 16 does not include the low pressure air supply ducts 114a and the low pressure air return ducts 118a, and only includes the moderate pressure air supply ducts 114b and the moderate pressure air return ducts 118b. By omitting the low pressure air supply ducts 114a and the low pressure air return ducts 118a, the moderate pressure air supply ducts 114b will supply all the purified air 30g to the interior airflow space 34, and the moderate pressure air return ducts 118b will return the recirculated air 30c from the interior airflow space 34. In yet another version, the air management system 16 does not include the moderate pressure air supply ducts 114b and the moderate pressure air return ducts 118b, and only includes the low pressure air supply ducts 114a and the low pressure air return ducts 118a. By omitting the moderate pressure air supply ducts 114b and the moderate pressure air return ducts 118b, the low pressure air supply ducts 114a will supply all the purified air 30g to the interior airflow space 34, and the low pressure air return ducts 118a will return the recirculated air 30c from the interior airflow space 34. With this version, if increased airflow rates are needed in the interior airflow space 34, for example, in aisleways, powerful fans with moderate pressure or high pressure airflow may be added at various locations in the interior airflow space 34, for example, placed at various locations along the aisleways. In yet another version, the air 30 in the low pressure air supply ducts 114a and the air 30 in the moderate pressure air supply ducts 114b mix together and flow into the interior airflow space 34, and the air 30 in the low pressure air return duct 118a and the air 30 in the moderate pressure air return duct 118b mix before entering into the compressor assembly 22.

As shown in FIG. 1B, the air purification and management system 10 further comprises the control system 18 to control the air purification system 14, and to control the air management system 16. As shown in FIG. 1B, in one version, the control system 18 comprises a centralized control system 18a coupled to the compressor assembly 22, the air supply duct assembly 112, and the air return duct assembly 116, via the connector elements 137, such as wired connectors or wireless connectors. In other versions, the control system 18 comprises a distributed control system 18b (see FIG. 16).

As shown in FIG. 1B, the air purification and management system 10 further comprises the plurality of sensors 20 comprising internal sensors 20a coupled to one or more of, the one or more interior airflow spaces 34 in the building 12, and coupled to one or more secondary interior locations 152 in the building 12. As further shown in FIG. 1B, the plurality of sensors 20 further comprise external sensors 20b coupled to one or more of, the external air intake duct 108, and one or more exterior portions 154 of the building 12.

The exemplary air purification and management system 10 shown in FIG. 1B provides a contamination mitigation 128 (see FIG. 16) for occupants 100 (see FIGS. 1A, 16), such as human occupants 100a (see FIGS. 1A, 16), of the building 12, such as the office building 12a, against contamination by one or more of, the one or more biological agents 42 (see FIGS. 1A, 16), and the one or more chemical agents 44 (see FIGS. 1A, 16).

Now referring to FIG. 1C, FIG. 1C is an illustration of a perspective view of the air purification and management system 10 and the building 12 of FIG. 1B, showing sensors 20, such as external sensors 20b, in solid circles in solid lines. In one version, as shown in FIG. 1C, the external sensors 20b are coupled to, and located at, the external air intake ducts 108, and are coupled to, and located at, exterior portions 154 of the building 12, including coupled to, or located at, the building skin 138, coupled near the building entrance 142, and coupled to, or located at, the rooftop 145. In other versions, the external sensors 20b may be coupled to, and located at, other exterior portions 154 (see FIG. 1C) of the building 12. The external sensors 20b and the sensor control network 130 (see FIGS. 1A, 16) sense an external environment 155a (see FIG. 1C) external to the building 12. The external sensors 20b and the sensor control network 130 sense normal operations and adjust to normal changes outside the building 12. The external sensors 20b and the sensor control network 130 further sense temperature, humidity, solar insolation, and other environmental conditions in the external environment 155a. As used herein, “insolation” means the measure of solar radiation energy received on a given surface area in a given time, typically expressed in watts per square meter.

The external sensors 20b and the sensor control network 130 further monitor external threats, such as the presence of biological agents 42 that are harmful, and chemical agents 44 that are harmful. The control system 18 and the sensor control network 130 manage resources to counter the external threats, such as the presence of the biological agents 42 and the chemical agents 44.

The external sensors 20b may comprise temperature sensors, including semiconductor-based sensors, thermocouple sensors, resistance temperature detectors, and negative temperature coefficient thermistors; humidity sensors, including capacitive sensors, resistive sensors, and thermal sensors; pressure sensors; solar insolation sensors; motion sensors to detect occupants 100, such as human occupants 100a, outside of the building 12; contact sensors to indicate whether a duct or valve external to the building 12 is open or closed; sound sensors to indicate sounds outside the building 12; air quality sensors to monitor toxins, dust, nitrogen dioxide, ozone, and other substances in the external air 30a (see FIG. 16) outside the building 12; infrared (IR) sensors; sensors that sense or detect biological agents 42 and chemical agents 44, and other suitable external sensors 20b.

Now referring to FIG. 1D, FIG. 1D is an illustration of a perspective view of the air purification and management system 10 and the building 12 of FIG. 1B, showing internal sensors 20a in open circles in solid lines coupled to, and within, the exemplary interior airflow space 34 in the building 12, and coupled to, and within, secondary interior locations 152 in the building 12.

As shown in FIG. 1D, the internal sensors 20a are coupled to, and located at, and within, the interior airflow space 34. The interior airflow space 34 or spaces may include conference rooms 34a (see FIGS. 1B, 1K), cubicles, office cubicles, offices, aisleways, restrooms, auditoriums, elevators, cafeterias, lunch rooms, or other suitable interior airflow spaces 34. As further shown in FIG. 1D, the internal sensors 20a are coupled to, and located at, and within, secondary interior locations 152, such as non-airflow spaces of the building 12, including in closets and storage areas on each floor 140, in the basement 144, and other suitable secondary interior locations 152 within the building 12. The internal sensors 20a and the sensor control network 130 sense an internal environment 155b (see FIG. 1D) internal to the building 12. The internal sensors 20a and the sensor control network 130 sense normal operations, and manage the moderate air pressure 115b of the moderate pressure air supply ducts 114b (see FIG. 1B) and the moderate pressure air return ducts 118b (see FIG. 1B), to control airflow 31 (see FIG. 16) of the air 30 (see FIGS. 1A, 16), such as the purified air 30g (see FIGS. 1A, 16) and the recirculated air 30c (see FIGS. 1A, 16), in interior airflow spaces 34, such as conference rooms 34a, cubicles, office cubicles, offices, aisleways, restrooms, auditoriums, elevators, cafeterias, lunch room, or other suitable interior airflow spaces 34.

Further, the internal sensors 20a and the sensor control network 130 enable direct-path-to-return airflow 124, while minimizing energy consumption. In addition, the internal sensors 20a and the sensor control network 130 sense where occupants 100, such as human occupants 100a, are and adjust the airflow 31 to maintain respiratory safety.

The internal sensors 20a and the sensor control network 130 further monitor internal threats, such as the presence of biological agents 42 that are harmful, for example, an internal sensor 20a that can detect if an occupant 100 is contaminated or infected with a biological agent 42, and chemical agents 44 that are harmful. The control system 18 and the sensor control network 130 manage resources to counter the internal threats, such as the presence of the biological agents 42 and the chemical agents 44.

The internal sensors 20a may comprise temperature sensors, including semiconductor-based sensors, thermocouple sensors, resistance temperature detectors, and negative temperature coefficient thermistors; humidity sensors, including capacitive sensors, resistive sensors, and thermal sensors; pressure sensors; motion sensors to detect occupants 100, such as human occupants 100a, within the building 12; contact sensors to indicate whether a duct or valve is open or closed within the building 12; sound sensors to indicate sounds, for example, if an occupant 100 within the building 12 sneezes, such as within the interior airflow space 34; air quality sensors that monitor toxins, dust, nitrogen dioxide, ozone, and other substances in the internal air 30e within the building 12; sensors that sense or detect biological agents 42 and chemical agents 44, and other suitable internal sensors 20a.

Now referring to FIG. 1E, FIG. 1E is an illustration of a perspective view of the air purification and management system 10 and the building 12 of FIG. 1B, showing two external air intake ducts 108 in solid lines. As shown in FIG. 1E, the two external air intake ducts 108 comprise the first external air intake duct 108a and the second external air intake duct 108b. In one version, as shown in FIG. 1E, each external air intake duct 108 has the first end opening 110a, or intake end opening, that can be opened and closed to the external environment 155a with one or more duct valves 107. As shown in FIG. 1E, each external air intake duct 108 has the first end opening 110a located at the rooftop 145 of the building 12 and exposed to the external air 30a. In one version, as shown in FIG. 1E, each external air intake duct 108 has the second end opening 110b, or exit end opening, that can be opened and closed to the air intake assembly 24 (see FIGS. 1A-1B) of the compressor assembly 22 (see FIGS. 1A-1B), via one or more duct valves 107. Each external air intake duct 108 is preferably in the form of a cylindrical tube 156 (see FIG. 1E) that is preferably closed or sealed along the length of the external air intake duct 108. As shown in FIG. 1E, the first external air intake duct 108a and the second external air intake duct 108b both have a length that extends from above the rooftop 145, through each floor 140 of the building 12 and into the basement 144 of the building 12.

As shown in FIG. 1E, in one version, each of the first external air intake duct 108a and the second external air intake duct 108b have duct valves 107 at each of the first end opening 110a, and at each of the second end opening 110b, that can open and close to allow, or not allow, the intake of air 30 (see FIG. 16), such as the external air 30a (see FIG. 16), for example, ambient air 30b (see FIG. 16), including contaminated air 30d, into the first external air intake duct 108a and the second external air intake duct 108b. In other versions, the duct valves 107 may be positioned at another location along the length and within the interior of the cylindrical tube 156 of each of the first external air intake duct 108a and the second external air intake duct 108b.

The air 30 (see FIG. 16), such as the external air 30a (see FIG. 16), or ambient air 30b, (see FIG. 16), enters the building 12 through a limited number of external air intake ducts 108. Preferably, there is more than one external air intake duct 108. If one of the external air intake ducts 108 is compromised by threat of a biological agent 42 and/or a chemical agent 44, or destroyed by warfare or an attack, or disabled or sabotaged in some way, such external air intake duct 108 can be closed, such as closing of a duct valve 107, and air 30, such as the external air 30a, or ambient air 30b, is obtained from the remaining external air intake duct 108, or ducts. It is advantageous to space the first external air intake duct 108a and the second external air intake duct 108b a sufficient distance from each other. An environmental threat such as by threat of a biological agent 42 and/or a chemical agent 44, or threat by warfare or attack or sabotage, is less likely to compromise one or more of the external air intake ducts 108, if they are located a sufficient distance away from each other.

Now referring to FIG. 1F, FIG. 1F is an illustration of a perspective view of the air purification and management system 10 and the building 12 of FIG. 1B, showing in solid lines the compressor assembly 22, such as in the form of the multi-cylinder compressor assembly 94. As discussed above, and as shown in FIG. 1F, the compressor assembly 22 comprises the air intake assembly 24, the compressors 50 coupled to the air intake assembly 24, the plenum 60 coupled to the compressors 50, the decompressors 90 coupled to the plenum, and the air outlet assembly 26 coupled to the decompressors 90. The components of the multi-cylinder compressor assembly 94 are discussed in further detail below with regard to FIG. 13A.

The compressor assembly 22 supplies purified air 30g (see FIGS. 1A, 16) to the building 12, and in particular, to the air management system 16 (see FIGS. 1A-1B) housed in the building 12. The compressor assembly 22 is configured to intake, and can intake, external air 30a, or ambient air 30b, from the external environment 155a (see FIG. 1C), including contaminated air 30d contaminated with one or more of, one or more biological agents 42, and one or more chemical agents 44, and is configured to intake, and can intake recirculated air 30c (see FIGS. 1A, 16) from the one or more interior airflow spaces 34 (see FIGS. 1A-1B). The compressor assembly 22 outputs the purified air 30g to the air management system 16, and in particular, to the plurality of air supply ducts 114 (see FIGS. 1A-1B), where the purified air 30g has been purified or cleaned by deactivating or destroying one or more of, the one or more biological agents 42, and the one or more chemical agents 44. A pressure ratio (PR) 160 (see FIG. 16) of how much air 30 is being compressed in the compressor assembly 22 may be measured. As used herein, “pressure ratio” means the ratio of a discharge pressure to an inlet pressure. A compression ratio (CR) 161 (see FIG. 16) is a maximum pressure ratio.

Preferably, the compressor assembly 22 is located in a safe location, for example, in the basement 144 (see FIGS. 1F, 16), in an underground area 148 (see FIG. 16), on the rooftop 145 (see FIGS. 2A, 16), on a ground area 146 (see FIG. 16), in a structure 150 (see FIG. 16) that is adjacent or nearby the building 12, in another building 12, or in another suitable safe location. The building 12 may also have more than one compressor assembly 22. For example, a first compressor assembly 22a (see FIG. 16) coupled to the building 12 may supply hot air or warmed air, and a second compressor assembly 22b (see FIG. 16) coupled to the building 12 may supply cold air or cooled air (see FIG. 16), and the hot air or warmed air, and the cold air or cooled air, may be mixed, for example, in a common duct coupled to both the first compressor assembly 22a and the second compressor assembly 22b. In some versions, the cold air or cooled air, and the hot air or warmed air, may be mixed at the interior airflow space 34. In addition, elevated temperatures 52a (see FIG. 16), such as high temperatures 52c (see FIG. 16), may be achieved simply by changing a compression ratio 161 (see FIG. 16). The compression ratio (CR) 161 (see FIG. 16) can be adjusted with the timing 72 (see FIG. 16) of the opening of the plenum inlet valve 64. The compression ratio 161 is easily adjustable in real time. As used herein, “compression ratio” means a maximum pressure ratio and is the ratio between a volume in a cylinder with a piston at its highest point, e.g., top center of a stroke, and a volume with the piston at its lowest point, e.g., bottom center of the stroke, for example, a compression ratio of 3:1 means a ratio of an original volume of air in a cylinder or compression chamber to a compressed volume of air in the cylinder or compression chamber. In one version, the stroke need not be adjusted, but just the timing of when the air is allowed to escape from the cylinder. The 3:1 compression ratio is achieved by the valves opening, so that the air is allowed to escape when the volume is one-third of the original volume. The 10:1 compression ratio is achieved by the valves opening, so that the air is allowed to escape when the volume is one-tenth of the original volume. To avoid the situation where the air could be infinitely compressed if the piston can be designed to match perfectly flush with the cylinder end, a pressure release valve can be used so that the piston cannot overpressure the cylinder assembly. Alternatively, the compression ratio may be varied by moving the axle of the wheel the piston end is attached to closer or further from the cylinder head. Alternatively, the piston rod can be made shorter, so that an infinite compression ratio is not possible.

Now referring to FIG. 1G, FIG. 1G is an illustration of a perspective view of the air purification and management system 10 and the building 12 of FIG. 1B, showing a plurality of air supply ducts 114, such as low pressure air supply ducts 114a, in solid lines. Preferably, in one version, each of the plurality of low pressure air supply ducts 114a has a low air pressure 115a (see FIG. 16) in a range of from 0.002 psi (point zero zero two pound per square inch) to 0.05 psi (point zero five pound per square inch). In other versions, the low air pressure 115a may be less than 0.002 psi or greater than 0.05 psi.

The low pressure air supply ducts 114a supply most of the air 30, such as the purified air 30g, to the building interior 104a (see FIG. 1G) of the building 12. Preferably, the location of the low pressure air supply ducts 114a is near a floor level 140b (see FIG. 1G) on each floor 140 of the building 12, or mid-height level for lateral airflow on each floor 140 of the building 12. The plurality of air return ducts 118 (see FIGS. 1A-1B) in the ceiling facilitate the airflow 31 (see FIG. 16) from the floor level 140b to the ceiling. The controlled airflow pattern 125 (see FIG. 16) forces the air 30 from a contaminated occupant 100b (see FIGS. 1A, 16), or an infected occupant or person, upward from the floor level 140b or mid-height level, to the ceiling, which makes it less likely to be breathed or inhaled by another occupant 100 of the building 12.

As shown in FIG. 1G, each low pressure air supply duct 114a is preferably in the form of a cylindrical tube 156 that is preferably closed or sealed along the length of the low pressure air supply duct 114a. As shown in FIG. 1G, in one version, the low pressure air supply ducts 114a may have one or more duct valves 107 in or at each of a first end opening 162a, and/or a second end opening 162b, that may be opened and closed to allow or not allow the intake of the air 30, such as the purified air 30g. In other versions, the one or more duct valves 107 may be positioned at another location along the interior length of the cylindrical tube 156 of each low pressure air supply duct 114a.

Now referring to FIG. 1H, FIG. 1H is an illustration of a perspective view of the air purification and management system 10 and the building 12 of FIG. 1B, showing a plurality of air supply ducts 114, such as moderate pressure air supply ducts 114b, in solid lines. Preferably, in one version, each of the plurality of moderate pressure air supply ducts 114b has a moderate air pressure 115b in a range of from 0.05 psi (point zero five pound per square inch) to 5.0 psi (five pounds per square inch). In other versions, the moderate air pressure 115b may be less than 0.05 psi or greater than 5.0 psi.

The moderate pressure air supply ducts 114b supply the air 30, such as the purified air 30g, to the building interior 104a (see FIG. 1H) of the building 12, and to selected portions of the building interior 104a, such as the one or more interior airflow spaces 34, at selected times. The internal sensors 20a (see FIG. 1D) detect certain movement and locations of occupants 100, such as human occupants 100a, in the building interior 104a (see FIG. 1H). Surge airflow 31a (see FIG. 16) is surged in these locations with a controlled airflow pattern 125 (see FIG. 16) designed to move contaminated air 30d from a contaminated occupant 100b, or infected person, to the air return duct assembly 116 (see FIGS. 1A-1B), without any other occupant 100 being in the path of the surge airflow 31a. After the occupant 100 has exited the interior airflow space 34, the surge airflow 31a is turned off, and the surge airflow 31a is returned to a normal airflow 31b (see FIG. 16) from the low pressure air supply ducts 114a (see FIG. 1G).

The airflow 31, such as the surge airflow 31a, preferably has, in one version, a velocity 29 (see FIG. 16), or airflow speed, in a range of 3 ft/sec (three feet per second) to 5 ft/sec (five feet per second) in the interior airflow space 34. In other versions, the velocity 29, or airflow speed, of the surge airflow 31a is greater than 5 ft/sec (five feet per second).

In the air supply ducts 114, the velocity 29 (see FIG. 16) of the airflow 31, or airflow speed) is 10 (ten) times to 20 (twenty) times higher or greater. The velocity 29, or airflow speed, of the airflow 31 may be reduced if the air supply ducts 114 comprise a significant proportion of an inter-floor volume of the floor level 140b of each floor 140.

As shown in FIG. 1H, each moderate pressure air supply duct 114b is preferably in the form of a cylindrical tube 156 that is preferably closed or sealed along the length of the moderate pressure air supply duct 114b. The diameter of the cylindrical tube 156 of the moderate pressure air supply duct 114b is preferably smaller than the diameter of the cylindrical tube 156 of the low pressure air supply duct 114a. As shown in FIG. 1H, in one version, the moderate pressure air supply ducts 114b may have one or more duct valves 107 at each of a first end opening 164a, and/or a second end opening 164b, that may be opened and closed to allow or not allow the intake of the air 30, such as the purified air 30g. In other versions, the one or more duct valves 107 may be positioned at another location along the interior length of the cylindrical tube 156 of each moderate pressure air supply duct 114b.

Now referring to FIG. 1I, FIG. 1I is an illustration of a perspective view of the air purification and management system 10 and the building 12 of FIG. 1B, showing a plurality of air return ducts 118, such as low pressure air return ducts 118a, in solid lines. Preferably, in one version, each of the plurality of low pressure air return ducts 118a has a low air pressure 115a in a range of from 0.002 psi (point zero zero two pound per square inch) to 0.05 psi (point zero five pound per square inch). In other versions, the low air pressure 115a may be less than 0.002 psi or greater than 0.05 psi. The low pressure air return ducts 118a collect most of the internal air 30e, including internal air 30e contaminated with one or more of, the one or more biological agents 42, and the one or more chemical agents 44, from the building interior 104a, including the interior airflow space interior 104b (see FIG. 1I) of the one or more interior airflow spaces 34, and returns it as recirculated air 30c to the compressor assembly 22.

The velocity 29 (see FIG. 16), or the airflow speed, of the airflow 31 (see FIG. 16) in the low pressure air return ducts 118a and in the interior airflow spaces 34 is preferably less than 0.5 ft/sec (zero point five feet per second). Preferably, the internal air 30e comprising contaminated air 30d and/or purified air 30g is introduced near the floor level 140b (see FIG. 1I) on each floor 140 of the building 12, or at a mid-height level for lateral airflow on each floor 140 of the building 12. Return air vents 165 (see FIG. 16) in the ceiling enforce the airflow 31 from the floor level 140b or the mid-height level, to the ceiling. The controlled airflow pattern 125 (see FIG. 16) forces the contaminated air 30d from a contaminated occupant 100b (see FIG. 16), or infected person, upward to the ceiling, which makes it less likely to be breathed or inhaled by another occupant 100 in the interior airflow space 34.

As shown in FIG. 1I, each low pressure air return duct 118a is preferably in the form of a cylindrical tube 156 that is preferably closed or sealed along the length of the low pressure air return duct 118a. In one version, as shown in FIG. 1I, the low pressure air return ducts 118a may have one or more duct valves 107 at each of a first end opening 166a, and/or a second end opening 166b, that may be opened and closed to allow or not allow the intake of the air 30, such as the recirculated air 30c. In other versions, the one or more duct valves 107 may be positioned at another location along the interior length of the cylindrical tube 156 of each low pressure air return duct 118a.

In one version, the interior 120a (see FIG. 1I) of one or more of the plurality of low pressure air return ducts 118a (see FIG. 1I) contains one or more ultraviolet lights 122 (see FIGS. 1I, 16). The one or more ultraviolet lights 122 may be optionally used and located in the low pressure air return ducts 118a (see FIG. 1I), to aid in deactivating or destroying one or more of the one or more biological agents 42 and/or one or more of the chemical agents 44. As an alternative to the ultraviolet lights 122, or in addition to the ultraviolet lights 122, the low pressure air return ducts 118a may have one or more grates with heated wires inside the low pressure air return ducts 118a, or another suitable device or assembly to aid in deactivating or destroying one or more of the one or more biological agents 42 and/or one or more of the chemical agents 44.

Now referring to FIG. 1J, FIG. 1J is an illustration of a perspective view of the air purification and management system 10 and the building 12 of FIG. 1B, showing a plurality of air return ducts 118, such as moderate pressure air return ducts 118b, in solid lines. Preferably, in one version, each of the plurality of moderate pressure air return ducts 118b has a moderate air pressure 115b in a range of from 0.05 psi (point zero five pound per square inch) to 5.0 psi (five pounds per square inch). In other versions, the moderate air pressure 115b may be less than 0.05 psi or greater than 5.0 psi. The moderate pressure air return ducts 118b are the complement of the moderate pressure air supply ducts 114b. The function of the moderate pressure air supply ducts 114b and the moderate pressure air return ducts 118b are that they enforce the controlled airflow pattern 125 (see FIG. 16) from the air supply duct openings 114c (see FIGS. 1A-1B) to the air return duct openings 118c (see FIGS. 1A-1B). Without the air return ducts 118, the purified air 30g from the moderate pressure air supply ducts 114b does not have a place to go. The controlled airflow pattern 125 prevents contaminated air 30d breathed by a contaminated occupant 100b, such as an infected person, from being breathed by another occupant 100 in the interior airflow space 34 of the building 12. The moderate pressure air supply duct 114b or vent, and the moderate pressure air return duct 118b or vent, function as two nodes between which the air 30 will flow. The air 30 will flow from the higher-pressure location to the lower-pressure location. If there is no lower-pressure location that is not in effect to suck the air 30 in, the air 30 from the higher-pressure location will dissipate, thus preventing the function of that aspect of the air purification and management system 10.

The velocity 29 (see FIG. 16), or airflow speed, of the airflow 31 (see FIG. 16) coming from the air supply ducts 114, such as the moderate pressure air supply ducts 114b, matches the velocity 29 of the airflow 31 going to, and into, the air return ducts 118, such as the moderate pressure air return ducts 118b. The airflow 31 flowing to the air return ducts 118, such as the moderate pressure air return ducts 118b, stops when the air supply duct openings 114c (see FIGS. 1A-1B) are closed or turned off, and/or when one or more duct valves 107 in the air supply ducts 114, such as the moderate pressure air supply ducts 114b, are closed.

For example, surge airflow 31a (see FIG. 16) is surged in the one or more interior airflow spaces 34 with the controlled airflow pattern 125 (see FIG. 16) designed to move contaminated air 30d from the contaminated occupant 100b, or infected person, to the air return ducts 118 (see FIGS. 1A-1B), without any other occupant 100 being in the path of the surge airflow 31a. After the occupant 100 or occupants 100 has/have exited the interior airflow space 34, the surge airflow 31a (see FIG. 16) is turned off, and the surge airflow 31a is returned to a normal airflow 31b (see FIG. 16) from the low pressure air supply ducts 114a (see FIG. 1G).

The velocity 29, or airflow speed, of the airflow 31, such as the surge airflow 31a, of the purified air 30g and the internal air 30e in the one or more interior airflow spaces 34 coming from the moderate pressure air supply ducts 114b and flowing to the moderate pressure air return ducts 118b is preferably, in one version, in a range of 3 ft/sec (three feet per second) to 5 ft/sec (five feet per second). In other versions, the velocity 29, or airflow speed, of the airflow 31, such as the surge airflow 31a, of the purified air 30g and the internal air 30e in the one or more interior airflow spaces 34 may be greater than 5 ft/sec (five feet per second). The velocity 29 of the airflow 31, or airflow speed, of the purified air 30g and the internal air 30e in the one or more interior airflow spaces 34 coming from the moderate pressure air supply ducts 114b and flowing to the moderate pressure air return ducts 118b may operate in a slight vacuum at a pressure slightly less than the ambient pressure, and may be reduced if the air supply ducts 114, such as the moderate pressure air supply ducts 114b, comprise a substantial portion of an inter-floor volume.

As shown in FIG. 1J, each moderate pressure air return duct 118b is preferably in the form of a cylindrical tube 156 that is preferably closed or sealed along the length of the moderate pressure air return duct 118b. The diameter of the cylindrical tube 156 of the moderate pressure air return duct 118b is preferably smaller than the diameter of the cylindrical tube 156 of the low pressure air return duct 118a. As shown in FIG. 1J, in one version, the moderate pressure air return ducts 118b may have one or more duct valves 107 at each of a first end opening 168a, and/or a second end opening 168b, that may be opened and closed to allow or not allow the intake of the air 30, such as the recirculated air 30c. In other versions, the one or more duct valves 107 may be positioned at another location along the interior length of the cylindrical tube 156 of each moderate pressure air supply duct 114b.

In one version, the interior 120b (see FIG. 1J) of one or more of the plurality of moderate pressure air return ducts 118b (see FIG. 1I), contain one or more ultraviolet lights 122 (see FIGS. 1J, 16). The one or more ultraviolet lights 122 may be optionally used and located in the air return ducts 118 (see FIGS. 1I, 1J), such as the moderate pressure air return ducts 118b, to aid in deactivating or destroying one or more of the one or more biological agents 42 and/or one or more of the chemical agents 44. As an alternative to the ultraviolet lights 122, or in addition to the ultraviolet lights 122, the air return ducts 118, such as the moderate pressure air return ducts 118b, may have one or more grates with heated wires inside the air return ducts 118, such as the moderate pressure air return ducts 118b, or another suitable device or assembly to aid in deactivating or destroying one or more of the one or more biological agents 42 and/or one or more of the chemical agents 44.

Now referring to FIG. 1K, FIG. 1K is an illustration of a perspective view of the air purification and management system 10 and the building 12 of FIG. 1B, showing an exemplary interior airflow space 34, in solid lines, located in the building 12. As shown in FIG. 1K, in one version, the interior airflow space 34 comprises a conference room 34a. In other versions, the interior airflow spaces 34 may comprise cubicles, office cubicles, offices, aisleways, restrooms, auditoriums, elevators, cafeterias, lunch rooms, or other suitable interior airflow spaces 34. The interior airflow spaces 34 are designed for direct-path-to-return airflow 124 (see FIG. 16). As discussed above, the direct-path-to-return airflow 124 is an airflow field designed and achieved for which there is a very low probability of an occupant 100, such as a human occupant 100a, of the building 12, breathing air 30, such as contaminated air 30d, that has already been breathed by another occupant 100, such as another human occupant 100a, nearby in the interior airflow space 34, and in turn, any other occupant 100, such as any other human occupant 100a, in the building 12 in any other interior airflow space 34. Each type of interior airflow space 34 is designed to create the controlled airflow pattern 125 (see FIG. 16) to protect the occupants 100 of the building 12 from being contaminated or infect by another occupant 100 of the building 12.

As shown in FIG. 1K, the air supply ducts 114, such as a low pressure air supply duct 114a and a moderate pressure air supply duct 114b, originate from, and are coupled to, the compressor assembly 22, and terminate at, and are coupled to the interior airflow space 34. As further shown in FIG. 1K, the air return ducts 118, such as the low pressure air return duct 118a and the moderate pressure air return duct 118b, originate from, and are coupled to, the interior airflow space 34, and terminate at, and are coupled to the compressor assembly 22.

Now referring to FIGS. 2A-2H, FIGS. 2A-2H show illustrations of a side view of a version of an air purification system 14 of the disclosure that may be used in the air purification and management system 10 of the disclosure. FIGS. 2A-2H show the air purification system 14 comprising the compressor assembly 22, such as in the form of the reciprocating compressor assembly 92, in various positions of a compression-decompression cycle 170. FIG. 2A is an illustration of a side view of a version of the air purification system 14 of the disclosure with the compressor assembly 22, in the form of a reciprocating compressor assembly 92, in a first cycle position 170a. FIG. 2B is an illustration of a side view of the air purification system 14 with the compressor assembly 22, in the form of the reciprocating compressor assembly 92, of FIG. 2A, in a second cycle position 170b. FIG. 2C is an illustration of a side view of the air purification system 14 with the compressor assembly 22, in the form of the reciprocating compressor assembly 92, of FIG. 2A, in a third cycle position 170c. FIG. 2D is an illustration of a side view of the air purification system 14 with the compressor assembly 22, in the form of the reciprocating compressor assembly 92, of FIG. 2A, in a fourth cycle position 170d. FIG. 2E is an illustration of a side view of the air purification system 14 with the compressor assembly 22, in the form of the reciprocating compressor assembly 92, of FIG. 2A, in a fifth cycle position 170e. FIG. 2F is an illustration of a side view of the air purification system 14 with the compressor assembly 22, in the form of the reciprocating compressor assembly 92, of FIG. 2A, in a sixth cycle position 170f. FIG. 2G is an illustration of a side view of the air purification system 14 with the compressor assembly 22, in the form of the reciprocating compressor assembly 92, of FIG. 2A, in a seventh cycle position 170g. FIG. 2H is an illustration of a side view of the air purification system 14 with the compressor assembly 22, in the form of the reciprocating compressor assembly 92, of FIG. 2A, in an eighth cycle position 170h. The compression-decompression cycle 170 of FIGS. 2A-2H, with the first cycle position 170a (see FIG. 2A), the second cycle position 170b (see FIG. 2B), the third cycle position 170c (see FIG. 2C), the fourth cycle position 170d (see FIG. 2D), the fifth cycle position 170e (see FIG. 2E), the sixth cycle position 170f (see FIG. 2F), the seventh cycle position 170g (see FIG. 2G), and the eighth cycle position 170h (see FIG. 2H), is one example of the number of cycle positions in the compression-decompression cycle 170 to illustrate an example of the air purification system 14. However, the compression-decompression cycle 170 may be depicted with more than eight cycle positions or with two to seven cycle positions. Such cycle positions shown in FIGS. 2A-2H are merely views of the rotation of the compressor crankshaft 175a and the decompressor crankshaft 175b, which is a continuous motion.

In one version, as shown in FIGS. 2A-2H, the compressor assembly 22, such as the reciprocating compressor assembly 92, is positioned on a rooftop 145 of a building 12, such as a commercial building 12b (see FIG. 2A), where the building 12 is a new build building 12d (see FIG. 2A). In other versions, the building 12 may comprise an office building 12a and/or a retrofitted building 12c. As shown in FIGS. 2A-2H, the building interior 104a (see FIG. 2A) of the building 12 has interior airflow spaces 34 in communication with an air supply duct 114, via air supply duct openings 114c (see FIG. 2A).

As further shown in FIGS. 2A-2H, the compressor assembly 22, such as the reciprocating compressor assembly 92, comprises the air intake assembly 24 with air intake openings 27 coupled to the compressor 50, the plenum 60 coupled to the compressor 50, the decompressor 90 coupled to the plenum 60, and the air outlet assembly 26 coupled to the decompressor 90. The compressor assembly 22 supplies the purified air supply 70 (see FIGS. 2H, 16) of purified air 30g (see FIGS. 2H, 16) to the air management system 16 (see FIG. 2H). The air outlet assembly 26 (see FIG. 2H) directs the purified air 30g (see FIG. 2H) out of the compressor assembly 22 (see FIG. 2H) and into the air management system 16 (see FIG. 2H).

As shown in FIGS. 2A-2H, the air purification system 14 comprising the compressor assembly 22, such as the reciprocating compressor assembly 92, has stages 171 of heating and cooling during the compression-decompression cycle 170. As shown in FIGS. 2A-2H, one stage 171 includes a compression and heating stage 171a in the compressor 50 having ambient air 30b, including contaminated air 30d contaminated with biological agents 42 (see FIGS. 2A-2C) and chemical agents 44 (see FIGS. 2A-2C) that is compressed to obtain compressed air 30f. As further shown in FIGS. 2A-2H, another stage 171 includes a high temperature dwell stage 171b of the compressed air 30f, in the plenum 60, at the elevated temperature 52a (see FIG. 16) for the effective dwell time 58a (see FIG. 16), to purify the compressed air 30f, by deactivating or destroying the biological agents 42 (see FIG. 2D) and the chemical agents 44 (see FIG. 2D), present in the compressed air 30f, to obtain the purified air 30g. As further shown in FIGS. 2A-2H, another stage 171 includes a decompression and cooling stage 171c of the compressed air 30f, to decompress and thereby cool the compressed air 30f, in the decompressor 90, to obtain decompressed air 30h, that is cooled for supplying within the building 12.

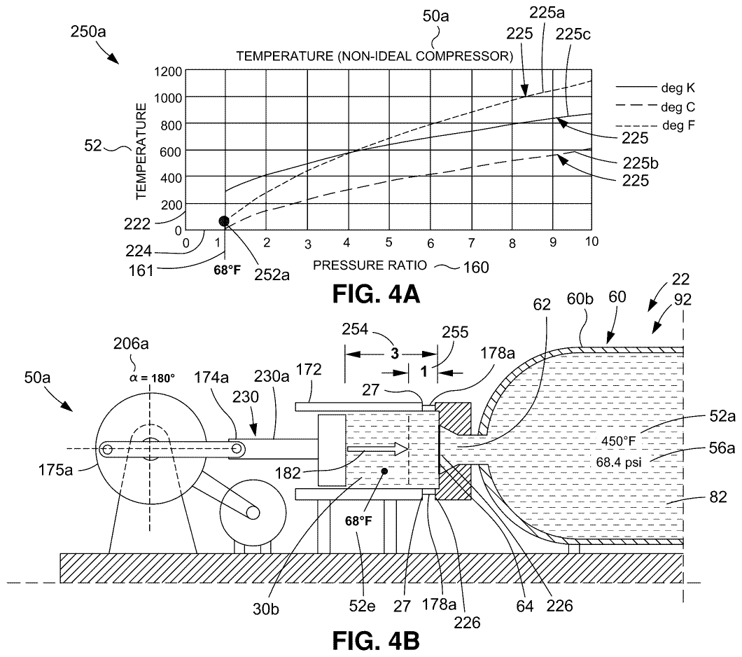

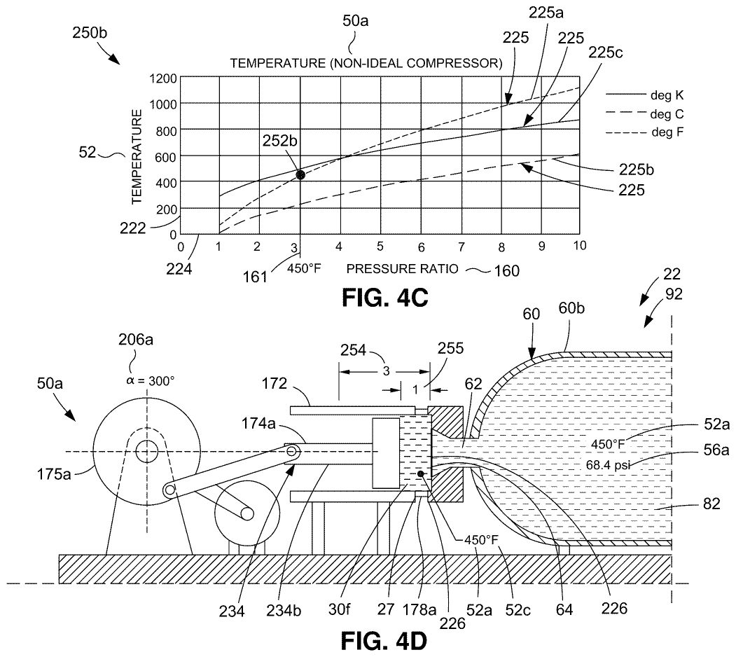

As shown in FIG. 2A, the at least one compressor 50 comprises a compression chamber 172 with a piston 174, such as a compressor piston 174a, coupled to a crankshaft 175, such as a compressor crankshaft 175a, and the compressor crankshaft 175a is coupled to a motor 176, such as an electric motor 176a. As shown in FIG. 2A, the compression chamber 172 has the air intake openings 27, with valves 178, such as compressor valves 178a, that may be opened and closed to allow, or not allow, the intake of the ambient air 30b, including the contaminated air 30d. FIG. 2A shows the compressor valves 178a at an end of the compression chamber 172 near the plenum inlet 62 of the plenum 60. In other versions, the compressor valves 178a may be positioned at another location along the compression chamber 172. As shown in FIG. 2A, preferably, the compression chamber 172 is in the form of a cylinder 180, such as a compression cylinder 180a. The compressor piston 174a is configured to move, and moves, within the compression chamber interior, and the compressor piston 174a moves forward in a compressing direction 182 (see FIG. 4B) to compress the ambient air 30b, including contaminated air 30d contaminated with biological agents 42 (see FIGS. 2A-2C) and the chemical agents 44 (see FIGS. 2A-2C). The compressor piston 174a is further configured to retract, and retracts, back in a retracting direction opposite the compressing direction 182, to take in the ambient air 30b, including the contaminated air 30d, to start the compression-decompression cycle 170 again.

As further shown in FIG. 2F, the piston 174, such as the compressor piston 174a, has a piston head 185a coupled to, or integrated with, a piston body 186a. As shown in FIG. 2F, the piston body 186a is coupled to a connecting rod 194a, via a first pivot pin 196a. As further shown in FIG. 2F, the connecting rod 194a is connected to the compressor crankshaft 175a, via a second pivot pin 200a. The compressor crankshaft 175a is coupled to a drive belt 202a (see FIG. 2F) of the electric motor 176a (see FIG. 2F). The electric motor 176a drives the compressor crankshaft 175a. In one version, the drive mechanism to drive the compressor crankshaft 175a comprises the motor 176, such as the electric motor 176a. In other versions, the drive mechanism may comprise linear actuators, hydraulic actuators, electromagnets, or another suitable drive mechanism that creates linear motion. As further shown in FIG. 2F, the compressor crankshaft 175a is coupled to a support structure 204a that is coupled to, or sits upon, the top surface of the rooftop 145, and the decompressor crankshaft 175b is coupled to a support structure 204b that is coupled to, or sits upon, the top surface of the rooftop 145. As further shown in FIGS. 2A-2H, the compressor crankshaft 175a has a rotation position 206, such as an alpha (α) rotation position 206a, shown at various degrees depending on the rotation of the compressor crankshaft 175a, via the connecting rod 194a and the electric motor 176a.

As shown in FIGS. 2A-2H, in one version, the at least one plenum 60 comprises a vacuum-jacketed plenum tank 60b, for example, a high-temperature plenum tank 60c (see FIG. 2A). As shown in FIG. 2A, the plenum 60 has the plenum inlet 62 coupled to the compression chamber 172, and the plenum inlet 62 has the plenum inlet valve 64 that opens and closes to allow the compressed air 30f from the compression chamber 172 into the interior 82 of the plenum 60. As further shown in FIG. 2A, the plenum 60 has the plenum exit 66 coupled to the decompressor 90, and the plenum exit 66 has the plenum exit valve 68 that opens and closes to allow the purified air 30g from the interior 82 of the plenum 60 into the decompressor 90. As further shown in FIG. 2A, the plenum 60 has plenum supports 208 coupled to the bottom of the plenum 60 and designed to keep the plenum 60 raised above the top surface of the rooftop 145 and not directly sitting on the top surface of the rooftop 145. The plenum 60 may have one or more plenum supports 208 depending on the size and length of the plenum 60.

The timing 72 (see FIG. 16) of the plenum inlet valve 64 opening is adjustable, to regulate the elevated temperature 52a (see FIG. 16) and the elevated pressure 56a (see FIG. 16) in the interior 82 of the plenum 60. The compressor geometry 76 (see FIG. 16) of the compressor 50 and the plenum geometry 78 (see FIG. 16) of the plenum 60 are fixed. In addition, a compression ratio 161 (see FIG. 16), discussed in further detail below, can be adjusted with the timing 72 (see FIG. 16) of the opening of the plenum inlet valve 64 (see FIG. 4B) of the plenum 60 (see FIG. 4B).

The plenum 60 is configured to maintain the compressed air 30f comprising the ambient air 30b and contaminated air 30d at the elevated temperature 52a (see FIG. 16) for the effective dwell time 58a (see FIG. 16), to purify the compressed air 30f, by deactivating or destroying the biological agents 42 (see FIG. 2D) and the chemical agents 44 (see FIG. 2D), present in the compressed air 30f, to obtain the purified air 30g.

As shown in FIGS. 2A-2H, in one version, the at least one decompressor 90 comprises a decompression chamber 210 with a piston 174 (see FIG. 2A), such as a decompressor piston 174b, coupled to a crankshaft 175 (see FIG. 2A), such as a decompressor crankshaft 175b, and the decompressor crankshaft 175b is coupled to a generator 212 (see FIG. 2A).

As shown in FIG. 2A, the decompression chamber 210 has air outlet supply openings 38 with valves 178, such as decompressor valves 178b, that may be opened and closed to allow, or not allow, the exit of the purified air 30g, for example, the decompressed air 30h that is cooled, from the decompression chamber 210 into the ducts 106 inside the building 12, and into the interior airflow spaces 34 within the building 12. FIG. 2A shows, in one version, the decompressor valves 178b at an end of the decompression chamber 210 near the plenum exit 66 of the plenum 60. In other versions, the decompressor valves 178b may be positioned at another location along the decompression chamber 210.

As shown in FIG. 2A, preferably, the decompression chamber 210 is in the form of a cylinder 180, such as a decompression cylinder 180b, having a decompression chamber interior 214. The decompressor piston 174b is configured to move, and moves, within the decompression chamber interior 214.

When the purified air 30g, under high pressure 56c (see FIG. 16) and heat 54 (see FIG. 16), exits the plenum 60 and enters the decompression chamber interior 214, the purified air 30g that is under high pressure 56c and heat 54 causes the decompressor piston 174b to move back in a decompressing direction 216 (see FIGS. 2F, 2G, 2H), to decompress the purified air 30g, to obtain the decompressed air 30h, within the decompression chamber 210. The high pressure 56c from the decompressor piston 174b moves the decompressor piston 1174b, and the purified air 30g decompresses as it moves. Thus, the decompressor 90 harnesses the energy stored in the plenum 60, such as the pressurized plenum, which is beneficial, since that energy can be used to supply the motor 176 for the compressor 50 side of the compressor assembly 22. There is still some energy that needs to be supplied to the compressor assembly 22 to make it work, but at least the energy from the decompressor 90 can offset some of the energy required to compress the air 30 before it goes into the plenum 60. After the purified air 30g is cooled and decompressed to obtain the decompressed air 30h, the decompressor piston 174b moves forward in a return direction 218 (see FIGS. 2B, 2C, 2D), to expel the purified air 30g, for example, the decompressed air 30h, that becomes cooled, through the air outlet supply openings 38, when the decompressor valves 178b are open, and out of the decompression chamber 210, and into the ducts 106 inside the building 12, and into the interior airflow spaces 34 within the building 12.

As further shown in FIG. 2F, the piston 174, such as the decompressor piston 174b, has a piston head 185b coupled to, or integrated with, a piston body 186b. As shown in FIG. 2A, the piston body 186b is coupled to, or integrated with, the piston head 185b, and is coupled to a connecting rod 194b, via a first pivot pin 196b. As further shown in FIG. 2F, the connecting rod 194b is connected to the decompressor crankshaft 175b, via a second pivot pin 200b. The decompressor crankshaft 175b is coupled to a drive belt 202b (see FIG. 2F) of the generator 212 (see FIG. 2F). The decompressor crankshaft 175b drives the generator 212, via the drive belt 202b, to generate electricity to recapture energy. As further shown in FIGS. 2A-2H, the decompressor crankshaft 175b has a rotation position 206, such as a beta (β) rotation position 206b, shown at various degrees depending on the rotation of the decompressor crankshaft 175b, via the connecting rod 194b.

As further shown in FIGS. 2A-2E, a graph 220, such as a compression and heating graph 220a, in the compression and heating stage 171a, shows a y-axis 222 of temperature 52 versus an x-axis 224 of a pressure ratio 160 for the compressor 50 at various positions during the compression-decompression cycle 170. As shown in FIGS. 2A-2E, the compression and heating graph 220a shows plots 225, including a degree Fahrenheit plot 225a, a degree Celsius plot 225b, and a degree Kelvin plot 225c, and the compression and heating graph 220a shows a compression ratio 161.

As further shown in FIGS. 2E-2H, a graph 220, such as a decompression and cooling graph 220b, in the decompression and cooling stage 171c, shows the y-axis 222 of temperature 52 versus the x-axis 224 of a pressure ratio 160 for the decompressor 90 at various positions during the compression-decompression cycle 170. As shown in FIGS. 2E-2H, the decompression and cooling graph 220b shows plots 225, including a degree Fahrenheit plot 225a, a degree Celsius plot 225b, and a degree Kelvin plot 225c, and the decompression and cooling graph 220b shows a compression ratio 161.

Now referring to FIG. 2A, FIG. 2A shows the compressor assembly 22, in the form of the reciprocating compressor assembly 92, in a first cycle position 170a of the compression-decompression cycle 170, such as a cycle position 0 (zero). FIG. 2A shows ambient air 30b at an ambient temperature 52e (see also FIG. 16) of 68 °F (sixty-eight degrees Fahrenheit) in the atmosphere 102, external to the compressor assembly 22. FIG. 2A further shows the compression chamber 172 full of ambient air 30b and contaminated air 30d contaminated with a biological agent 42 and a chemical agent 44. The ambient air 30b in the compression chamber 172 is at ambient temperature 52e of 68 °F (sixty-eight degrees Fahrenheit) and is at a constant pressure 56d (see FIG. 16). FIG. 2A shows the air intake openings 27 and the compressor valves 178a closed in a closed position 226. FIG. 2A further shows the rotation position 206, such as the alpha (α) rotation position 206a, of the compressor crankshaft 175a, at 180° (one-hundred eighty degrees). FIG. 2A further shows the compressor piston 174a in the compression chamber 172 in a retracted position 230, such as a fully retracted position 230a.

FIG. 2A further shows the graph 220, such as the compression and heating graph 220a, in the compression and heating stage 171a, with the y-axis 222 of temperature 52 versus the x-axis 224 of pressure ratio 160 for the compressor 50 in the first cycle position 170a, such as the cycle position 0 (zero). As shown in FIG. 2A, the compression and heating graph 220a shows plots 225, including the degree Fahrenheit plot 225a, the degree Celsius plot 225b, and the degree Kelvin plot 225c. As shown in FIG. 2A, the compression and heating graph 220a in the first cycle position 170a, such as the cycle position 0 (zero), shows the temperature 52 of 68 °F (sixty-eight degrees Fahrenheit) (20 °C (twenty degrees Celsius), 293 K (two-hundred ninety-three Kelvin)) and a compression ratio 161 of 1:1.

As shown in FIG. 2A, in the first cycle position 170a, such as the cycle position 0 (zero), and in the high temperature dwell stage 171b, the plenum 60, such as the vacuum-jacketed plenum tank 60b, has an elevated temperature 52a of 450 °F (four-hundred fifty degrees Fahrenheit) (232 °C (two-hundred thirty-two degrees Celsius)) and a pressure 56 of 68.4 psi (pounds per square inch) in the interior 82 of the plenum 60. FIG. 2A further shows the plenum inlet 62 and the plenum inlet valve 64 closed in the closed position 226, and shows the plenum exit 66 and the plenum exit valve 68 closed in the closed position 226.

As shown in FIG. 2A, in the first cycle position 170a, such as the cycle position 0 (zero), and in the decompression and cooling stage 171c, purified air 30g, previously received from the plenum 60, and decompressed into decompressed air 30h, that is cooled, is expelled from the decompression chamber 210 into the ducts 106 inside the building 12, and into the interior airflow spaces 34 within the building 12. FIG. 2A shows the air outlet supply openings 38 and the decompressor valves 178b of the decompression chamber 210 open in an open position 228, to allow the purified air 30g, such as decompressed air 30h, that is cooled, to flow out of the decompression chamber 210 and into the ducts 106 inside the building 12, and into the interior airflow spaces 34 within the building 12.

The purified air 30g, such as decompressed air 30h, is cooled in the decompression chamber 210 to ambient temperature 52e of 68 °F (sixty-eight degrees Fahrenheit) and is at a constant pressure 56d (see FIG. 16), and the purified air 30g, such as the decompressed air 30h, cooled to ambient temperature 52e of 68 °F (sixty-eight degrees Fahrenheit) is expelled into the ducts 106 inside the building 12, and into the interior airflow spaces 34 within the building 12. The purified air 30g, such as the decompressed air 30h, that is cooled, may also be expelled to secondary interior locations 152 (see FIG. 1A) in the building 12. FIG. 2A further shows the rotation position 206, such as the beta (β) rotation position 206b of the decompressor crankshaft 175b, at 30°(thirty degrees). FIG. 2A further shows the decompressor piston 174b in the decompression chamber 210 in a return position 232, such as a first return position 232a.

Now referring to FIG. 2B, FIG. 2B shows the compressor assembly 22, in the form of the reciprocating compressor assembly 92, in a second cycle position 170b of the compression-decompression cycle 170. FIG. 2B shows the ambient air 30b, at ambient temperature 52e (see also FIG. 16) of 68 °F (sixty-eight degrees Fahrenheit) in the atmosphere 102, external to the compressor assembly 22. FIG. 2B further shows the compression chamber 172 full of ambient air 30b, and also having contaminated air 30d contaminated with the biological agent 42 and the chemical agent 44. The ambient air 30b and the contaminated air 30d in the compression chamber 172 is being compressed, and thereby heated, with the compressor piston 174a, to an intermediate temperature 52f (see also FIG. 16), of 209 °F (two-hundred nine degrees Fahrenheit) (98 °C (ninety-eight degrees Celsius)) that is higher than the ambient temperature 52e, and is at an intermediate pressure 56e (see FIG. 16) that is higher than the constant pressure 56d of the ambient air 30b. FIG. 2B shows the air intake openings 27 and the compressor valves 178a closed in the closed position 226. FIG. 2B shows the position of the compressor piston 174a in the compression chamber 172 moved a distance toward the plenum inlet 62, as compared to the compressor piston 174a position in the first cycle position 170a. FIG. 2B further shows the compressor piston 174a in the compression chamber 172 in a compression position 234, such as a first compression position 234a. FIG. 2B further shows the rotation position 206, such as the alpha (α) rotation position 206a, of the compressor crankshaft 175a, at 270° (two-hundred seventy degrees).

FIG. 2B further shows the graph 220, such as the compression and heating graph 220a, in the compression and heating stage 171a, with the y-axis 222 of temperature 52 versus the x-axis 224 of pressure ratio 160 for the compressor 50 in the second cycle position 170b. As shown in FIG. 2B, the compression and heating graph 220a shows plots 225, including the degree Fahrenheit plot 225a, the degree Celsius plot 225b, and the degree Kelvin plot 225c. As shown in FIG. 2B, the compression and heating graph 220a in the second cycle position 170b, shows the temperature 52 of 209 °F (two-hundred nine degrees Fahrenheit) (98 °C (ninety-eight degrees Celsius), 371 K (three-hundred seventy-one Kelvin)) and a compression ratio 161 of 1.65:1.

As shown in FIG. 2B, in the second cycle position 170b, and in the high temperature dwell stage 171b, the plenum 60, such as the vacuum-jacketed plenum tank 60b, has an elevated temperature 52a of 450 °F (four-hundred fifty degrees Fahrenheit) and an elevated pressure 56a (see FIG. 16) of 68.4 psi (pounds per square inch) in the interior 82 of the plenum 60. FIG. 2B further shows the plenum inlet 62 and the plenum inlet valve 64 closed in the closed position 226, and shows the plenum exit 66 and the plenum exit valve 68 closed in the closed position 226.

As shown in FIG. 2B, in the second cycle position 170b, and in the decompression and cooling stage 171c, purified air 30g, previously received from the plenum 60, and decompressed into decompressed air 30h, that is cooled, continues to be expelled from the decompression chamber 210 into the ducts 106 inside the building 12, and into the interior airflow spaces 34 within the building 12. FIG. 2B shows the air outlet supply openings 38 and the decompressor valves 178b of the decompression chamber 210 open in the open position 228, and shows the decompressor piston 174b in the decompression chamber 210 moved a distance toward the plenum exit 66, to continue to allow the purified air 30g, such as decompressed air 30h, that is cooled, to flow out of the decompression chamber 210 and into the ducts 106 inside the building 12, and into the interior airflow spaces 34 within the building 12. FIG. 2B further shows the decompressor piston 174b in the decompression chamber 210 in a return position 232, such as a second return position 232b.

The purified air 30g, such as decompressed air 30h, has been cooled in the decompression chamber 210 to ambient temperature 52e of 68 °F (sixty-eight degrees Fahrenheit) and is at a constant pressure 56d (see FIG. 16), and the purified air 30g, such as the decompressed air 30h that has been cooled to ambient temperature 52e of 68 °F (sixty-eight degrees Fahrenheit) is expelled into the ducts 106 inside the building 12, and into the interior airflow spaces 34 within the building 12. The purified air 30g, such as the decompressed air 30h, that is cooled may also be expelled to secondary interior locations 152 (see FIG. 1A). FIG. 2B further shows the rotation position 206, such as the beta (β) rotation position 206b of the decompressor crankshaft 175b, at 120° (one-hundred twenty degrees).

Now referring to FIG. 2C, FIG. 2C shows the compressor assembly 22, in the form of the reciprocating compressor assembly 92, in a third cycle position 170c of the compression-decompression cycle 170. FIG. 2C shows the ambient air 30b, at the ambient temperature 52e (see also FIG. 16) of 68 °F (sixty-eight degrees Fahrenheit) in the atmosphere 102, external to the compressor assembly 22. FIG. 2C further shows the ambient air 30b and the contaminated air 30d contaminated with the biological agent 42 and the chemical agent 44 fully compressed, and thereby heated, by the compressor piston 174a, to a maximum compression in the compression chamber 172, to obtain compressed air 30f. FIG. 2C shows the compressor piston 174a in the compression chamber 172 in a compression position 234, such as second compression position 234b, and shows that the compression of the ambient air 30b and the contaminated air 30d in the compression chamber 172 has reached its maximum value.

As shown in FIG. 2C, the compressed air 30f is compressed, and thereby heated, to an elevated temperature 52a (see also FIG. 16), such as a high temperature 52c (see also FIG. 16), of 450 °F (four-hundred fifty degrees Fahrenheit) that is higher than the ambient temperature 52e and higher than the intermediate temperature 52f, and is at an elevated pressure 56a (see FIG. 16), such as a high pressure 56c (see FIG. 16), that is higher than the constant pressure 56d (see FIG. 16) and higher than the intermediate pressure 56e (see FIG. 16). FIG. 2C shows the air intake openings 27 and the compressor valves 178a closed in the closed position 226. FIG. 2C further shows the rotation position 206, such as the alpha (α) rotation position 206a, of the compressor crankshaft 175a, at 300° (three-hundred degrees).

FIG. 2C further shows the graph 220, such as the compression and heating graph 220a, in the compression and heating stage 171a, with the y-axis 222 of temperature 52 versus the x-axis 224 of pressure ratio 160 for the compressor 50 in the third cycle position 170c. As shown in FIG. 2C, the compression and heating graph 220a shows plots 225, including the degree Fahrenheit plot 225a, the degree Celsius plot 225b, and the degree Kelvin plot 225c. As shown in FIG. 2C, the compression and heating graph 220a in the third cycle position 170c, shows the temperature 52 of 450 °F (four-hundred fifty degrees Fahrenheit) (232 °C (two-hundred thirty-two degrees Celsius), 505 K (five-hundred five Kelvin)) and a compression ratio 161 of 3:1.

As shown in FIG. 2C, in the third cycle position 170c, and in the high temperature dwell stage 171b, the plenum 60, such as the vacuum-jacketed plenum tank 60b, has an elevated temperature 52a, such as a high temperature 52c, of 450 °F (four-hundred fifty degrees Fahrenheit) and an elevated pressure 56a (see FIG. 16) of 68.4 psi (pounds per square inch) in the interior 82 of the plenum 60. FIG. 2C further shows the plenum inlet 62 and the plenum inlet valve 64 closed in the closed position 226, and shows the plenum exit 66 and the plenum exit valve 68 closed in the closed position 226.

As shown in FIG. 2C, in the third cycle position 170c, and in the decompression and cooling stage 171c, purified air 30g, previously received from the plenum 60, and decompressed into decompressed air 30h, that is cooled, continues to be expelled from the decompression chamber 210 into the ducts 106 inside the building 12, and into the interior airflow spaces 34 within the building 12. FIG. 2C shows the air outlet supply openings 38 and the decompressor valves 178b of the decompression chamber 210 open in the open position 228, and the decompressor piston 174b in the decompression chamber 210 moved a further distance toward the plenum exit 66 than the distance in the second cycle position 170b, to continue to allow the purified air 30g, such as decompressed air 30h, that is cooled, to flow out of the decompression chamber 210 and into the ducts 106 inside the building 12, and into the interior airflow spaces 34 within the building 12.

The purified air 30g, such as decompressed air 30h, has been cooled in the decompression chamber 210 to ambient temperature 52e of 68 °F (sixty-eight degrees Fahrenheit) and is at a constant pressure 56d (see FIG. 16), and the purified air 30g, such as the decompressed air 30h that has been cooled to ambient temperature 52e of 68 °F (sixty-eight degrees Fahrenheit) is expelled into the ducts 106 inside the building 12, and into the interior airflow spaces 34 within the building 12. The purified air 30g, such as the decompressed air 30h, that is cooled may also be expelled to secondary interior locations 152 (see FIG. 1A). FIG. 2C further shows the rotation position 206, such as the beta (β) rotation position 206b of the decompressor crankshaft 175b, at 150° (one-hundred fifty degrees). FIG. 2C further shows the decompressor piston 174b in the decompression chamber 210 in a return position 232, such as a third return position 232c.

Now referring to FIG. 2D, FIG. 2D shows the compressor assembly 22, in the form of the reciprocating compressor assembly 92, in a fourth cycle position 170d of the compression-decompression cycle 170. FIG. 2D shows the plenum inlet 62 and the plenum inlet valve 64 open in the open position 228, to allow the compressed air 30f, which is hot at an elevated temperature 52a and a high temperature 52c of 450 °F (four-hundred fifty degrees Fahrenheit) and under a high pressure 56c (see FIG. 16), to flow into the interior 82 of the plenum 60. The plenum exit 66 and the plenum exit valve 68 remain closed in the closed position 226.

FIG. 2D shows the ambient air 30b, at the ambient temperature 52e (see also FIG. 16) of 68 °F (sixty-eight degrees Fahrenheit) in the atmosphere 102, external to the compressor assembly 22. FIG. 2D further shows the compressed air 30f contaminated with the biological agent 42 and the chemical agent 44 expelled from the compression chamber 172 into the interior 82 of the plenum 60, and shows the compressor piston 174a in the compression chamber 172 in a compression position 234, such as a third compression position 234c.

FIG. 2D shows the compressed air 30f in the compression chamber 172 and in the plenum 60 is at the elevated temperature 52a (see also FIG. 16), such as the high temperature 52c (see also FIG. 16), of 450 °F (four-hundred fifty degrees Fahrenheit), and is at the elevated pressure 56a (see FIG. 16), such as the high pressure 56c (see FIG. 16), such as 68.4 psi (sixty-eight point four pounds per square inch). FIG. 2D shows the air intake openings 27 and the compressor valves 178a closed in the closed position 226, and shows the plenum inlet 62 and the plenum inlet valve 64 in the open position 228, and the plenum exit 66 and the plenum exit valve 68 in the closed position 226. FIG. 2D further shows the rotation position 206, such as the alpha (α) rotation position 206a, of the compressor crankshaft 175a, at 330° (three-hundred thirty degrees).

FIG. 2D further shows the graph 220, such as the compression and heating graph 220a, in the compression and heating stage 171a, with the y-axis 222 of temperature 52 versus the x-axis 224 of pressure ratio 160 for the compressor 50 in the fourth cycle position 170d. As shown in FIG. 2D, the compression and heating graph 220a shows plots 225, including the degree Fahrenheit plot 225a, the degree Celsius plot 225b, and the degree Kelvin plot 225c. As shown in FIG. 2D, the compression and heating graph 220a in the fourth cycle position 170d, shows the temperature 52 of 450 °F (four-hundred fifty degrees Fahrenheit) and a compression ratio 161 of 3:1.

As shown in FIG. 2D, in the fourth cycle position 170d, and in the high temperature dwell stage 171b, the plenum 60, such as the vacuum-jacketed plenum tank 60b, has an elevated temperature 52a of 450 °F (four-hundred fifty degrees Fahrenheit) and an elevated pressure 56a (see FIG. 16) of 68.4 psi (pounds per square inch) in the interior 82 of the plenum 60. The compressed air 30f is maintained at the elevated temperature 52a (see also FIG. 16), such as the high temperature 52c (see also FIG. 16), of 450 °F (four-hundred fifty degrees Fahrenheit), and is maintained at the elevated pressure 56a (see FIG. 16), such as the high pressure 56c (see FIG. 16), for an effective dwell time 58a (see FIG. 16), to deactivate or destroy the biological agent 42 (see FIG. 2D) and the chemical agent 44 (see FIG. 2D) in the compressed air 30f, to obtain purified air 30g (see FIG. 2E) free of any biological agent 42 and chemical agent 44.

As shown in FIG. 2D, in the fourth cycle position 170d, and in the decompression and cooling stage 171c, purified air 30g, previously received from the plenum 60, and decompressed into decompressed air 30h, that is cooled, has been fully expelled from the decompression chamber 210 into the ducts 106 inside the building 12, and into the interior airflow spaces 34 within the building 12. FIG. 2D shows the air outlet supply openings 38 and the decompressor valves 178b of the decompression chamber 210 open in the open position 228, and shows the decompressor piston 174b in the decompression chamber 210 in a return position 232, such as a full return position 232d adjacent the plenum exit 66, which is in the closed position 226.

The purified air 30g, such as decompressed air 30h, has been cooled in the decompression chamber 210 to ambient temperature 52e of 68 °F (sixty-eight degrees Fahrenheit) and is at a constant pressure 56d (see FIG. 16), and has been expelled into the ducts 106 inside the building 12, and into the interior airflow spaces 34 within the building 12. The purified air 30g, such as the decompressed air 30h, that is cooled may also be expelled to secondary interior locations 152 (see FIG. 1A). FIG. 2D further shows the rotation position 206, such as the beta (β) rotation position 206b of the decompressor crankshaft 175b, at 180°(one-hundred eighty degrees).

Now referring to FIG. 2E, FIG. 2E shows the compressor assembly 22, in the form of the reciprocating compressor assembly 92, in a fifth cycle position 170e of the compression-decompression cycle 170, where after the compressed air 30f has been maintained in the plenum 60 both at the elevated temperature 52a, such as the high temperature 52c, of 450 °F (four-hundred fifty degrees Fahrenheit), and at the elevated pressure 56a (see FIG. 16), such as the high pressure 56c (see FIG. 16), for the effective dwell time 58a (see FIG. 16), such that the biological agent 42 (see FIG. 2A) and the chemical agent 44 (see FIG. 2A) are deactivated or destroyed, to obtain the purified air 30g, the plenum exit 66 and the plenum exit valve 68 are opened in the open position 228, to allow the purified air 30g, which is hot and at a high pressure 56c, to flow into the decompression chamber 210. FIG. 2E shows the plenum inlet 62 and the plenum inlet valve 64 open in the open position 228, to enable the compressed air 30f that is hot and at a high pressure 56c, to be pushed from the compression chamber 172 into the plenum 60.

FIG. 2E shows the ambient air 30b, at the ambient temperature 52e (see also FIG. 16) of 68 °F (sixty-eight degrees Fahrenheit) in the atmosphere 102, external to the compressor 50 of the compressor assembly 22. FIG. 2E further shows the compressor piston 174a in the compression chamber 172 in a compression position 234, such as a fully extended compression position 234d. FIG. 2E further shows the air intake openings 27 and the compressor valves 178a in the closed position 226. FIG. 2E further shows the rotation position 206, such as the alpha (α) rotation position 206a, of the compressor crankshaft 175a, at 0°(zero degrees).

FIG. 2E further shows the graph 220, such as the compression and heating graph 220a, in the compression and heating stage 171a, with the y-axis 222 of temperature 52 versus the x-axis 224 of pressure ratio 160 for the compressor 50 in the fifth cycle position 170e. As shown in FIG. 2E, the compression and heating graph 220a shows plots 225, including the degree Fahrenheit plot 225a, the degree Celsius plot 225b, and the degree Kelvin plot 225c. As shown in FIG. 2E, the compression and heating graph 220a in the fifth cycle position 170e, shows the temperature 52 of 450 °F (four-hundred fifty degrees Fahrenheit) and a compression ratio 161 of 3:1.

As shown in FIG. 2E, in the fifth cycle position 170e, and in the high temperature dwell stage 171b, the plenum 60, such as the vacuum-jacketed plenum tank 60b, has an elevated temperature 52a and a high temperature 52c of 450 °F (four-hundred fifty degrees Fahrenheit) and an elevated pressure 56a (see FIG. 16) of 68.4 psi (pounds per square inch) in the interior 82 of the plenum 60. Maintaining the compressed air 30f in the plenum 60 at the elevated temperature 52a, such as the high temperature 52c, of 450 °F (four-hundred fifty degrees Fahrenheit), and at the elevated pressure 56a, such as the high pressure 56c, for the effective dwell time 58a, effectively deactivates or destroys the biological agent 42 and the chemical agent 44, and purifies the compressed air 30f, to obtain the purified air 30g.

As shown in FIG. 2E, in the fifth cycle position 170e, and in the decompression and cooling stage 171c, the purified air 30g, enters or flows into the decompression chamber 210 from the plenum 60, and pushes the decompressor piston 174b away from the plenum exit 66. As shown in FIG. 2E, the purified air 30g in the decompression chamber 210 is at the elevated temperature 52a, such as the high temperature 52c, of 450 °F (four-hundred fifty degrees Fahrenheit), and at the elevated pressure 56a (see FIG. 16), such as the high pressure 56c (see FIG. 16). FIG. 2E shows the decompressor piston 174b in the decompression chamber 210 in a decompression position 235, such as a first decompression position 235a. FIG. 2E further shows the air outlet supply openings 38 and the decompressor valves 178b of the decompression chamber 210 closed in the closed position 226.

FIG. 2E shows the ambient air 30b, at the ambient temperature 52e (see also FIG. 16) of 68 °F (sixty-eight degrees Fahrenheit) in the ducts 106 inside the building 12, and in the interior airflow spaces 34 within the building 12. FIG. 2E further shows the rotation position 206, such as the beta (β) rotation position 206b of the decompressor crankshaft 175b, at 210° (two-hundred ten degrees).

FIG. 2E further shows another graph 220, such as a decompression and cooling graph 220b, in the decompression and cooling stage 171c, with the y-axis 222 of temperature 52 versus the x-axis 224 of pressure ratio 160 for the compressor 50 in the fifth cycle position 170e. As shown in FIG. 2E, the decompression and cooling graph 220b shows plots 225, including the degree Fahrenheit plot 225a, the degree Celsius plot 225b, and the degree Kelvin plot 225c. As shown in FIG. 2E, the decompression and cooling graph 220b in the fifth cycle position 170e, shows the temperature 52 of 450 °F (four-hundred fifty degrees Fahrenheit) and a compression ratio 161 of 3:1.

Now referring to FIG. 2F, FIG. 2F shows the compressor assembly 22, in the form of the reciprocating compressor assembly 92, in a sixth cycle position 170f of the compression-decompression cycle 170, where the desired volume of the purified air 30g has flowed from the plenum 60 into the decompression chamber 210 of the decompressor 90. As shown in FIG. 2F, the purified air 30g in the decompression chamber 210 is at the elevated temperature 52a, such as the high temperature 52c, of 450 °F (four-hundred fifty degrees Fahrenheit), and at the elevated pressure 56a (see FIG. 16), such as the high pressure 56c (see FIG. 16). FIG. 2F shows the air outlet supply openings 38 and the decompressor valves 178b of the decompression chamber 210 closed in the closed position 226. FIG. 2F shows the plenum exit 66 and the plenum exit valve 68 open in the open position 228, and shows the plenum inlet 62 and the plenum inlet valve 64 closed in the closed position 226.

FIG. 2F shows the ambient air 30b, at the ambient temperature 52e (see also FIG. 16) of 68 °F (sixty-eight degrees Fahrenheit) in the atmosphere 102, external to the compressor 50 of the compressor assembly 22. FIG. 2F further shows the compressor piston 174a in the compression chamber 172 in a retracted position 230, such as a first retracted position 230b. FIG. 2F further shows the air intake openings 27 and the compressor valves 178a in the open position 228. FIG. 2F further shows the rotation position 206, such as the alpha (α) rotation position 206a, of the compressor crankshaft 175a, at 30° (thirty degrees).

As shown in FIG. 2F, in the sixth cycle position 170f, and in the high temperature dwell stage 171b, the plenum 60, such as the vacuum-jacketed plenum tank 60b, has an elevated temperature 52a of 450 °F (four-hundred fifty degrees Fahrenheit) and an elevated pressure 56a (see FIG. 16) of 68.4 psi (pounds per square inch) in the interior 82 of the plenum 60.

As shown in FIG. 2F, in the sixth cycle position 170f, and in the decompression and cooling stage 171c, the desired volume of purified air 30g has entered or flowed into the decompression chamber 210 from the plenum 60, and pushes the decompressor piston 174b away from the plenum exit 66 even further. FIG. 2F shows the decompressor piston 174b in the decompression chamber 210 in a decompression position 235, such as a second decompression position 235b. FIG. 2F further shows the air outlet supply openings 38 and the decompressor valves 178b of the decompression chamber 210 closed in the closed position 226.

FIG. 2F shows the ambient air 30b, at the ambient temperature 52e (see also FIG. 16) of 68 °F (sixty-eight degrees Fahrenheit) in the ducts 106 inside the building 12, and in the interior airflow spaces 34 within the building 12. FIG. 2F further shows the rotation position 206, such as the beta (β) rotation position 206b of the decompressor crankshaft 175b, at 240° (two-hundred forty degrees).

FIG. 2F further shows the graph 220, such as the decompression and cooling graph 220b, in the decompression and cooling stage 171c, with the y-axis 222 of temperature 52 versus the x-axis 224 of pressure ratio 160 for the compressor 50 in the sixth cycle position 170f. As shown in FIG. 2F, the decompression and cooling graph 220b shows plots 225, including the degree Fahrenheit plot 225a, the degree Celsius plot 225b, and the degree Kelvin plot 225c. As shown in FIG. 2F, the decompression and cooling graph 220b in the sixth cycle position 170f, shows the temperature 52 of 450 °F (four-hundred fifty degrees Fahrenheit) and a compression ratio 161 of 3:1.

Now referring to FIG. 2G, FIG. 2G shows the compressor assembly 22, in the form of the reciprocating compressor assembly 92, in a seventh cycle position 170g of the compression-decompression cycle 170, where the plenum exit 66 and the plenum exit valve 68 are closed in the closed position 226, and the desired volume of the purified air 30g is in the decompression chamber 210 of the decompressor 90. The purified air 30g in the decompression chamber 210 is decreased slightly from the elevated pressure 56a, such as the high pressure 56c, but pushes the decompressor piston 174b even further away from the plenum exit 66, so that the purified air 30g expands in the decompression chamber 210 and cools to a temperature 52 of 209 °F (two-hundred nine degrees Fahrenheit). FIG. 2G shows the decompressor piston 174b in the decompression chamber 210 in a decompression position 235, such as a third decompression position 235c. FIG. 2G further shows the air outlet supply openings 38 and the decompressor valves 178b of the decompression chamber 210 closed in the closed position 226.

FIG. 2G shows the purified air 30g, at the ambient temperature 52e (see also FIG. 16) of 68 °F (sixty-eight degrees Fahrenheit) in the ducts 106 inside the building 12, and in the interior airflow spaces 34 within the building 12. FIG. 2G further shows the rotation position 206, such as the beta (β) rotation position 206b of the decompressor crankshaft 175b, at 270° (two-hundred seventy degrees).

FIG. 2G further shows the graph 220, such as the decompression and cooling graph 220b, in the decompression and cooling stage 171c, with the y-axis 222 of temperature 52 versus the x-axis 224 of pressure ratio 160 for the compressor 50 in the seventh cycle position 170g. As shown in FIG. 2G, the decompression and cooling graph 220b shows plots 225, including the degree Fahrenheit plot 225a, the degree Celsius plot 225b, and the degree Kelvin plot 225c. As shown in FIG. 2G, the decompression and cooling graph 220b in the seventh cycle position 170g, shows the temperature 52 of 209 °F (two-hundred nine degrees Fahrenheit) and a compression ratio 161 of 1.65:1.

FIG. 2G shows the ambient air 30b, at the ambient temperature 52e (see also FIG. 16) of 68 °F (sixty-eight degrees Fahrenheit) in the atmosphere 102, external to the compressor 50 of the compressor assembly 22. FIG. 2G further shows the compressor piston 174a in the compression chamber 172 in a retracted position 230, such as a second retracted position 230c. FIG. 2G further shows the air intake openings 27 and the compressor valves 178a in the open position 228. FIG. 2G further shows the rotation position 206, such as the alpha (α) rotation position 206a, of the compressor crankshaft 175a, at 60° (sixty degrees).

As shown in FIG. 2G, in the seventh cycle position 170g, and in the high temperature dwell stage 171b, the plenum 60, such as the vacuum-jacketed plenum tank 60b, has an elevated temperature 52a of 450 °F (four-hundred fifty degrees Fahrenheit) and a pressure 56 (see FIG. 16) of 68.4 psi (pounds per square inch) in the interior 82 of the plenum 60. FIG. 2G shows the plenum inlet 62 and the plenum inlet valve 64 in the closed position 226, and shows the plenum exit 66 and the plenum exit valve 68 in the closed position 226.

Now referring to FIG. 2H, FIG. 2H shows the compressor assembly 22, in the form of the reciprocating compressor assembly 92, in an eighth cycle position 170h of the compression-decompression cycle 170, where the purified air 30g in the decompression chamber 210 has been decompressed and cooled to a cooled temperature 52d (see FIG. 16), such as ambient temperature 52e of 68 °F (sixty-eight degrees Fahrenheit), and an ambient pressure, to obtain the decompressed air 30h (see also FIG. 16). As shown in FIG. 2H, the air outlet supply openings 38 and the decompressor valves 178b of the decompression chamber 210 are now open in the open position 228, so that the purified air 30g, such as the decompressed air 30h, can be expelled out of the decompression chamber 210 and into the ducts 106 inside the building 12, and into the interior airflow spaces 34 within the building 12. The purified air 30g, such as the decompressed air 30h, that is cooled, may also be expelled to secondary interior locations 152 (see FIG. 1A). FIG. 2H shows the decompressor piston 174b in the decompression chamber 210 in a decompression position 235, such as a full decompression position 235d. FIG. 2H further shows the rotation position 206, such as the beta (β) rotation position 206b of the decompressor crankshaft 175b, at 0° (zero degrees).

FIG. 2H further shows the graph 220, such as the decompression and cooling graph 220b, in the decompression and cooling stage 171c, with the y-axis 222 of temperature 52 versus the x-axis 224 of pressure ratio 160 for the compressor 50 in the eighth cycle position 170h. As shown in FIG. 2H, the decompression and cooling graph 220b shows plots 225, including the degree Fahrenheit plot 225a, the degree Celsius plot 225b, and the degree Kelvin plot 225c. As shown in FIG. 2H, the decompression and cooling graph 220b in the eighth cycle position 170h, shows the temperature 52 of 68 °F (sixty-eight degrees Fahrenheit) and a compression ratio 161 of 1:1.

FIG. 2H shows the ambient air 30b, at the ambient temperature 52e (see also FIG. 16) of 68 °F (sixty-eight degrees Fahrenheit) in the atmosphere 102, external to the compressor 50 of the compressor assembly 22. FIG. 2H further shows the compressor piston 174a in the compression chamber 172 in a retracted position 230, such as a third retracted position 230d. FIG. 2H further shows the air intake openings 27 and the compressor valves 178a in the open position 228. FIG. 2H further shows the rotation position 206, such as the alpha (α) rotation position 206a, of the compressor crankshaft 175a, at 150° (one-hundred fifty degrees).

As shown in FIG. 2H, in the eighth cycle position 170h, and in the high temperature dwell stage 171b, the plenum 60, such as the vacuum-jacketed plenum tank 60b, has an elevated temperature 52a of 450 °F (four-hundred fifty degrees Fahrenheit) and an elevated pressure 56a (see FIG. 16) of 68.4 psi (pounds per square inch) in the interior 82 of the plenum 60. FIG. 2H shows the plenum inlet 62 and the plenum inlet valve 64 in the closed position 226, and shows the plenum exit 66 and the plenum exit valve 68 in the closed position 226.

Now referring to FIG. 3A, FIG. 3A is an illustration of a side view of a version of an air purification system 14 of the disclosure with a compressor assembly 22, in the form of a reciprocating compressor assembly 92, having a plurality of baffles 80 positioned within the interior 82 of the plenum 60. As used herein, “baffle” means a flow-directing vane or panel used to direct a flow of air through a vessel, such as a tank, for example, a plenum tank. The plurality of baffles 80 force the compressed air 30f, that is at a high temperature 52c of 450 °F (four-hundred fifty degrees Fahrenheit) and a high pressure 56c (see FIG. 16) coming from the compression chamber 172, into and through a winding route 236 (see FIG. 3A) that is longer through the plenum 60 than if the compressed air 30f flowed through a plenum 60 without baffles 80. The baffles 80 help to ensure that the biological agents 42 (see FIGS. 2A, 16) and the chemical agents 44 (see FIGS. 2A, 16) in the compressed air 30f cannot easily travel directly from the plenum inlet 62 to the plenum exit 66, and thus allows a dwell time 58 (see FIG. 16), such as an effective dwell time 58a (see FIG. 16), to last a longer time. As shown in FIG. 3A, in one version, the baffles 80 comprise vane baffles 80a and are attached to interior portions 82a of the plenum 60 in an alternating arrangement 238. In other versions, the baffles 80 may be in another form and attached and arranged in another suitable manner.

FIG. 3A further shows the ambient air 30b, at the ambient temperature 52e of 68 °F (sixty-eight degrees Fahrenheit) in the atmosphere 102, external to the compressor assembly 22. As shown in FIG. 3A, in the stage 171, such as the compression and heating stage 171a, the compression chamber 172 contains contaminated air 30d contaminated with the biological agent 42 and the chemical agent 44. The contaminated air 30d in the compression chamber 172 is being compressed, and thereby heated, with the compressor piston 174a, to an intermediate temperature 52f of 209 °F (two-hundred nine degrees Fahrenheit) that is higher than the ambient temperature 52e, and is at an intermediate pressure 56e (see FIG. 16) that is higher than the constant pressure 56d (see FIG. 16) of the ambient air 30b. FIG. 3A shows an example depiction of a position of the compressor piston 174a in the compression chamber 172, such that the compression and resulting temperature equals 209 °F (two-hundred nine degrees Fahrenheit). FIG. 3A shows the air intake openings 27 and the compressor valves 178a closed in the closed position 226. FIG. 3A further shows the compressor piston 174a in the compression chamber 172 in a compression position 234, such as a first compression position 234a. FIG. 3A further shows the rotation position 206, such as the alpha (α) rotation position 206a, of the compressor crankshaft 175a, at 270° (two-hundred seventy degrees).

As further shown in FIG. 3A, in the stage 171, such as the high temperature dwell stage 171b, the plenum 60, such as the vacuum-jacketed plenum tank 60b, has an elevated temperature 52a of 450 °F (four-hundred fifty degrees Fahrenheit) and an elevated pressure 56 (see FIG. 16) of 68.4 psi (pounds per square inch) in the interior 82 of the plenum 60. FIG. 3A further shows the plenum inlet 62 and the plenum inlet valve 64 closed in the closed position 226, and shows the plenum exit 66 and the plenum exit valve 68 closed in the closed position 226.

As further shown in FIG. 3A, in the stage 171, such as the decompression and cooling stage 171c, purified air 30g, previously received from the plenum 60, and decompressed into decompressed air 30h, that is cooled, is expelled from the decompression chamber 210 into the ducts 106 inside the building 12, and into the interior airflow spaces 34 within the building 12. FIG. 3A shows the air outlet supply openings 38 and the decompressor valves 178b of the decompression chamber 210 open in the open position 228, and the decompressor piston 174b in the decompression chamber 210 in a return position 232, such as a second return position 232b.

The purified air 30g, such as decompressed air 30h, has been cooled in the decompression chamber 210 to ambient temperature 52e of 68 °F (sixty-eight degrees Fahrenheit) and is at a constant pressure 56d (see FIG. 16), and the purified air 30g, such as the decompressed air 30h, that has been cooled, to ambient temperature 52e of 68 °F (sixty-eight degrees Fahrenheit) is expelled into the ducts 106 inside the building 12, and into the interior airflow spaces 34 within the building 12. The purified air 30g, such as the decompressed air 30h, that is cooled, may also be expelled to secondary interior locations 152 (see FIG. 1A). FIG. 3A further shows the rotation position 206, such as the beta (β) rotation position 206b of the decompressor crankshaft 175b, at 120° (one-hundred twenty degrees).

Now referring to FIG. 3B, FIG. 3B is an illustration of a perspective view of a heat exchanger 84 that may be positioned and used in the interior 82 (see FIG. 3A) of a plenum 60 (see FIGS. 3A, 16) in a version of a compressor assembly 22 (see FIGS. 3A, 16), such as a reciprocating compressor assembly 92 (see FIG. 3A). As shown in FIG. 3B, the heat exchanger 84 has internal airflow passages 85 within and through a continuous tube 240 embedded in a plate 242. As further shown in FIG. 3B, compressed air 30f, such as contaminated air 30d, flows into an inlet 244, gets purified through the internal airflow passages 85 of the continuous baffle tube 240, and exits as purified air 30g through an outlet 246. The inlet 244 is preferably coupled to a cooling tower (not shown) on the rooftop 145 (see FIG. 3A) of the building 12, or in another location in or near the building 12. In one example, air 30 that is cooled from the cooling tower flows into the inlet 244, and through the internal airflow passages 85 of the heat exchanger 84 where it is heated. The outlet 246 is also preferably coupled to the cooling tower. The air 30 that is heated in the heat exchanger 84 may be cooled by flowing out the outlet 246 to the cooling tower.

Now referring to FIG. 3C, FIG. 3C is an illustration of a side view of a version of an air purification system 14 of the disclosure with a compressor assembly 22, in the form of a reciprocating compressor assembly 92, having a plenum 60 in the form of a coil 86. In this version, the plenum 60 is designed as a coil 86 (see FIG. 3C), which is different than the plenum 60 having baffles 80 in the interior 82 of the plenum 60 shown in FIG. 3A. As shown in FIG. 3C, the plenum 60 comprising the coil 86 has the plenum inlet 62 comprising a first coil end 248a coupled to the compression chamber 172, and has the plenum exit 66 comprising a second coil end 248b coupled to the decompression chamber 210. The coiled shape of the coil 86 helps to ensure that the biological agents 42 (see FIGS. 2A, 16) and the chemical agents 44 (see FIGS. 2A, 16) in the compressed air 30f cannot easily travel directly from the first coil end 248a to the second coil end 248b, and thus allows a dwell time 58 (see FIG. 16), such as an effective dwell time 58a (see FIG. 16), to last a longer time. The plenum 60 in the form of the coil 86 also provides a smaller radius of curvature for the plenum 60, or pressure vessel, which may enable thinner walls to be used to contain the internal pressure compared to the wall thickness required to contain the same pressure for the plenum variant. The plenum 60 in the form of the coil 86 may be supported at certain locations on the coil exterior by support structures (not shown).

FIG. 3C further shows ambient air 30b, at the ambient temperature 52e of 68 °F (sixty-eight degrees Fahrenheit) in the atmosphere 102, external to the compressor assembly 22. As shown in FIG. 3C, in the stage 171, such as the compression and heating stage 171a, the compression chamber 172 contains contaminated air 30d contaminated with the biological agent 42 and the chemical agent 44. The contaminated air 30d in the compression chamber 172 is being compressed, and thereby heated, with the compressor piston 174a, to an intermediate temperature 52f of 209 °F (two-hundred nine degrees Fahrenheit) that is higher than the ambient temperature 52e, and is at an intermediate pressure 56e (see FIG. 16) that is higher than the constant pressure 56d (see FIG. 16) of the ambient air 30b. FIG. 3C shows the air intake openings 27 and the compressor valves 178a closed in the closed position 226. FIG. 3C further shows the compressor piston 174a in the compression chamber 172 in a compression position 234, such as a first compression position 234a. FIG. 3C further shows the rotation position 206, such as the alpha (α) rotation position 206a, of the compressor crankshaft 175a, at 270° (two-hundred seventy degrees).

As further shown in FIG. 3C, in the stage 171, such as the high temperature dwell stage 171b, the plenum 60 in the form of the coil 86 has an elevated temperature 52a of 450 °F (four-hundred fifty degrees Fahrenheit) and a pressure 56 (see FIG. 16) of 68.4 psi (pounds per square inch) in the interior 82 of the plenum 60 in the form of the coil 86. FIG. 3C further shows the plenum inlet 62 comprising the first coil end 246a and the plenum inlet valve 64 closed in the closed position 226, and shows the plenum exit 66 comprising the second coil end 246b and the plenum exit valve 68 closed in the closed position 226.

As further shown in FIG. 3C, in the stage 171, such as the decompression and cooling stage 171c, purified air 30g, previously received from the plenum 60, and decompressed into decompressed air 30h, that is cooled, is expelled from the decompression chamber 210 into the ducts 106 inside the building 12, and into the interior airflow spaces 34 within the building 12. FIG. 3C shows the air outlet supply openings 38 and the decompressor valves 178b of the decompression chamber 210 open in the open position 228, and the decompressor piston 174b in the decompression chamber 210 in a return position 232, such as a second return position 232b.

The purified air 30g, such as decompressed air 30h, has been cooled in the decompression chamber 210 to ambient temperature 52e of 68 °F (sixty-eight degrees Fahrenheit) and is at a constant pressure 56d (see FIG. 16), and the purified air 30g, such as the decompressed air 30h, that has been cooled, to ambient temperature 52e of 68 °F (sixty-eight degrees Fahrenheit) is expelled into the ducts 106 inside the building 12, and into the interior airflow spaces 34 within the building 12. The purified air 30g, such as the decompressed air 30h, that is cooled, may also be expelled to secondary interior locations 152 (see FIG. 1A). FIG. 3A further shows the rotation position 206, such as the beta (β) rotation position 206b of the decompressor crankshaft 175b, at 120° (one-hundred twenty degrees).

Now referring to FIGS. 4A-4D, 5A-5D, and 6A-6D, FIGS. 4A-4D, 5A-5D, and 6A-6D show various pressure ratios 160 of various temperatures 52 of compressed air 30f in a non-ideal compressor 50a. To obtain different compression ratios 161, the hardware of the compressor assembly 22 does not need to change and can stay fixed. The compression ratio 161 (see FIGS. 4A, 4C, 5A, 5C, 6A, 6C) can be adjusted with the timing 72 (see FIG. 16) of the opening of the plenum inlet valve 64 (see FIG. 4B) of the plenum 60 (see FIG. 4B). Thus, the compression ratio 161 is governed by the timing 72, or when, the plenum inlet valve 64 is opened. In addition, the compression ratio 161 is easily adjustable in real time.

Now referring to FIG. 4A, FIG. 4A is an illustration of a graph 250a with the y-axis 222 of temperature 52 versus the x-axis 224 of pressure ratio 160 for a non-ideal compressor 50a. As shown in FIG. 4A, the graph 250a shows plots 225, including the degree Fahrenheit (deg F) plot 225a, the degree Celsius (deg C) plot 225b, and the degree Kelvin (deg K) plot 225c. FIG. 4A shows a plot point 252a at a temperature 52, such as ambient temperature 52e, of 68 °F (sixty-eight degrees Fahrenheit) (20 °C (twenty degrees Celsius), 293 K (two-hundred ninety-three K)) and a compression ratio 161 of 1:1.

Now referring to FIG. 4B, FIG. 4B is an illustration of a side view of a non-ideal compressor 50a, of a compressor assembly 22, such as a reciprocating compressor assembly 92, corresponding to the graph 250a in FIG. 4A, where the ambient air 30b in the compression chamber 172 of the non-ideal compressor 50a is at ambient temperature 52e of 68 °F (sixty-eight degrees Fahrenheit) and is at the compression ratio 161 of 1:1. As shown in FIG. 4B, an original volume 254 of ambient air 30b is shown by the number 3, and a compressed volume 255 of compressed air 30f (see FIG. 4D) is shown by the number 1. The compressed volume 255 (see also FIG. 4D) is ⅓ (one-third) of the original volume 254.

As shown in FIG. 4B, the compression chamber 172 contains ambient air 30b at ambient temperature 52e of 68 °F (sixty-eight degrees Fahrenheit) and is at a constant pressure 56d (see FIG. 16). FIG. 4B shows the air intake openings 27 and the compressor valves 178a closed in a closed position 226. FIG. 4B further shows the rotation position 206, such as the alpha (α) rotation position 206a, of the compressor crankshaft 175a, at 180° (one-hundred eighty degrees). FIG. 4B further shows the compressor piston 174a in the compression chamber 172 in a retracted position 230, such as a fully retracted position 230a, that corresponds to the compression ratio 161 of 1:1 in the graph 250b (see FIG. 4A). The compressor piston 174a is configured to move in the compression chamber 172 toward the plenum inlet 62 of the plenum 60 in the compressing direction 182 (see FIG. 4B).

As shown in FIG. 4B, the plenum 60, such as the vacuum-jacketed plenum tank 60b, has an elevated temperature 52a of 450 °F (four-hundred fifty degrees Fahrenheit) and an elevated pressure 56a of 68.4 psi (pounds per square inch) in the interior 82 of the plenum 60. FIG. 4B further shows the plenum inlet 62 and the plenum inlet valve 64 closed in the closed position 226.

Now referring to FIG. 4C, FIG. 4C is an illustration of a graph 250b with the y-axis 222 of temperature 52 versus the x-axis 224 of pressure ratio 160 for a non-ideal compressor 50a. As shown in FIG. 4C, the graph 250b shows plots 225, including the degree Fahrenheit (deg F) plot 225a, the degree Celsius (deg C) plot 225b, and the degree Kelvin (deg K) plot 225c. FIG. 4C shows a plot point 252b at a temperature 52 of 450 °F (four-hundred fifty degrees Fahrenheit) (232 °C (two-hundred thirty-two degrees Celsius), 505 K (five-hundred five Kelvin)) and a compression ratio 161 of 3:1.

Now referring to FIG. 4D, FIG. 4D is an illustration of a side view of a non-ideal compressor 50a, of a compressor assembly 22, such as a reciprocating compressor assembly 92, corresponding to the graph 250b in FIG. 4C, where the compressed air 30f in the compression chamber 172 of the non-ideal compressor 50a is at the elevated temperature 52a, for example, a high temperature 52c, of 450 °F (four-hundred fifty degrees Fahrenheit) and is at the compression ratio 161 of 3:1 in the graph 250b (see FIG. 4C). As shown in FIG. 4D, an original volume 254 of air 30, such as ambient air 30b (see FIG. 4B), is shown by the number 3, and the compressed volume 255 of air 30, such as compressed air 30f, is shown by the number 1. The compressed volume 255 is ⅓ (one-third) of the original volume 254.

As shown in FIG. 4D, the compressor piston 174a in the compression chamber 172 has moved in the compressing direction 182 (see FIG. 4B) to a compression position 234, such as second compression position 234b, and shows that the compression of the compressed air 30f in the compression chamber 172 has reached 3, which corresponds to the compression ratio 161 of 3:1 in the graph 250b (see FIG. 4C). As shown in FIG. 4D, the compressed air 30f is compressed, and thereby heated, to an elevated temperature 52a (see also FIG. 16), such as a high temperature 52c (see also FIG. 16), of 450 °F (four-hundred fifty degrees Fahrenheit) and is at an elevated pressure 56a (see FIG. 16), such as a high pressure 56c (see FIG. 16). FIG. 4D shows the air intake openings 27 and the compressor valves 178a closed in the closed position 226. FIG. 4D further shows the rotation position 206, such as the alpha (α) rotation position 206a, of the compressor crankshaft 175a, at 300° (three-hundred degrees).

As shown in FIG. 4D, the plenum 60, such as the vacuum-jacketed plenum tank 60b, has an elevated temperature 52a of 450 °F (four-hundred fifty degrees Fahrenheit) and an elevated pressure 56a of 68.4 psi (pounds per square inch) in the interior 82 of the plenum 60. FIG. 4D further shows the plenum inlet 62 and the plenum inlet valve 64 closed in the closed position 226. The plenum inlet valve 64 can be opened when the desired compression ratio 161 of 3:1 has been reached.

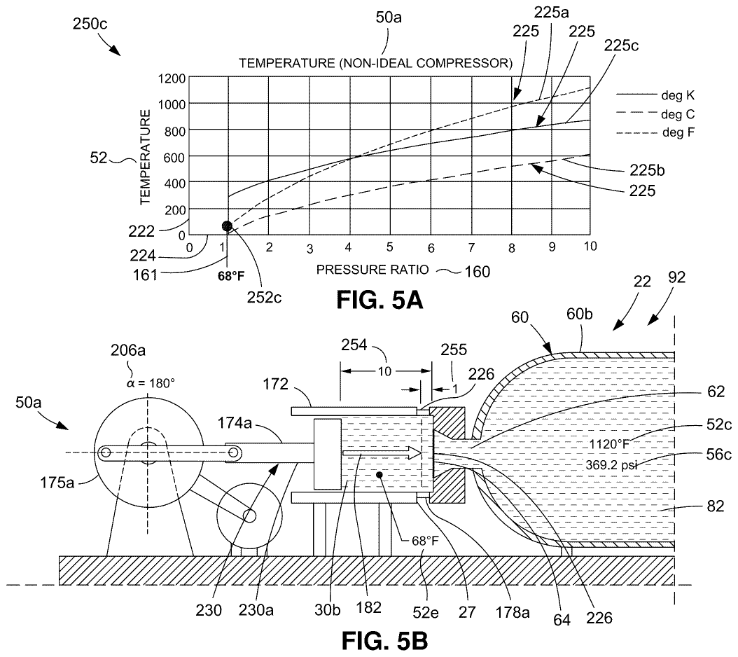

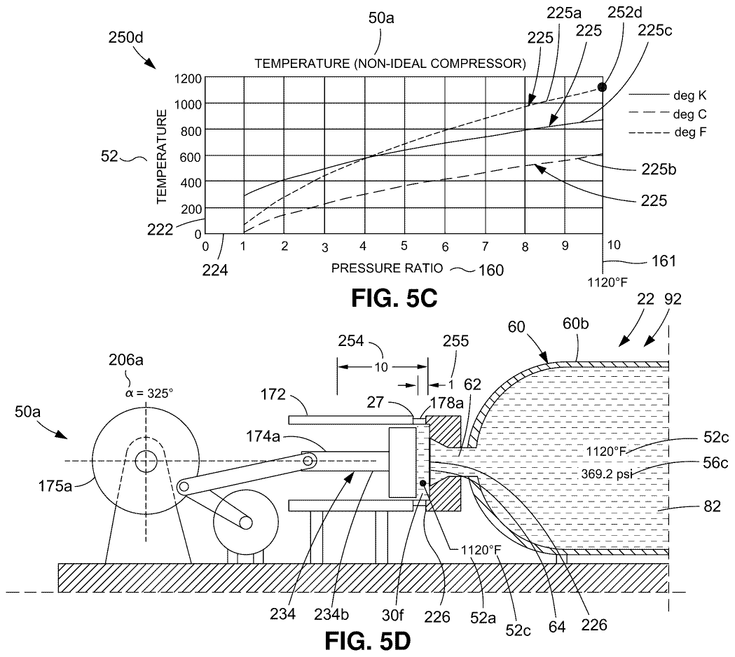

Now referring to FIG. 5A, FIG. 5A is an illustration of a graph 250c with the y-axis 222 of temperature 52 versus the x-axis 224 of pressure ratio 160 for a non-ideal compressor 50a. As shown in FIG. 5A, the graph 250c shows plots 225, including the degree Fahrenheit (deg F) plot 225a, the degree Celsius (deg C) plot 225b, and the degree Kelvin (deg K) plot 225c. FIG. 5A shows a plot point 252c at a temperature 52, such as ambient temperature 52e, of 68 °F (sixty-eight degrees Fahrenheit) and a compression ratio 161 of 1:1.

Now referring to FIG. 5B, FIG. 5B is an illustration of a side view of a non-ideal compressor 50a of a compressor assembly 22, such as a reciprocating compressor assembly 92, corresponding to the graph 250c in FIG. 5A, at ambient temperature 52e of 68 °F (sixty-eight degrees Fahrenheit) and the compression ratio 161 of 10:1 in the graph 250c (see FIG. 5A). As shown in FIG. 5B, the ambient air 30b, in the compression chamber 172 of the non-ideal compressor 50a is at ambient temperature 52e of 68 °F (sixty-eight degrees Fahrenheit) and is at the compression ratio 161 of 1:1. At higher compression ratios 161, higher temperatures are obtained. As shown in FIG. 5B, an original volume 254 of ambient air 30b is shown by the number 10, and a compressed volume 255 of compressed air 30f (see FIG. 5D) is shown by the number 1. The compressed volume 255 (see also FIG. 5D) is 1/10 (one-tenth) of the original volume 254.

As shown in FIG. 5B, the compression chamber 172 is full of air 30, such as ambient air 30b, at ambient temperature 52e of 68 °F (sixty-eight degrees Fahrenheit) and is at a constant pressure 56d (see FIG. 16). FIG. 5B shows the air intake openings 27 and the compressor valves 178a closed in the closed position 226. FIG. 5B further shows the rotation position 206, such as the alpha (α) rotation position 206a, of the compressor crankshaft 175a, at 180° (one-hundred eighty degrees). FIG. 5B further shows the compressor piston 174a in the compression chamber 172 in a retracted position 230, such as a fully retracted position 230a, that corresponds to the compression ratio 161 of 1:1 in the graph 250c (see FIG. 5A). The compressor piston 174a is configured to move in the compression chamber 172 toward the plenum inlet 62 of the plenum 60 in the compressing direction 182.

As shown in FIG. 5B, the plenum 60, such as the vacuum-jacketed plenum tank 60b, has a high temperature 52c of 1120 °F (one-thousand one-hundred twenty degrees Fahrenheit) (604 °C (six-hundred four degrees Celsius)) and a high pressure 56c of 369.2 psi (pounds per square inch) in the interior 82 of the plenum 60. FIG. 5B further shows the plenum inlet 62 and the plenum inlet valve 64 closed in the closed position 226.

Now referring to FIG. 5C, FIG. 5C is an illustration of a graph 250d with the y-axis 222 of temperature 52 versus the x-axis 224 of pressure ratio 160 for a non-ideal compressor 50a. As shown in FIG. 5C, the graph 250d shows plots 225, including the degree Fahrenheit (deg F) plot 225a, the degree Celsius (deg C) plot 225b, and the degree Kelvin (deg K) plot 225c. FIG. 5C shows a plot point 252d at a temperature 52 of 1120 °F (one-thousand one-hundred twenty degrees Fahrenheit) (604 °C (twenty degrees Celsius), 877 K (eight-hundred seventy-seven K)) and a compression ratio 161 of 10:1.

Now referring to FIG. 5D, FIG. 5D is an illustration of a side view of a non-ideal compressor 50a, of a compressor assembly 22, such as a reciprocating compressor assembly 92, corresponding to the graph 250d in FIG. 5C, where the compressed air 30f in the compression chamber 172 of the non-ideal compressor 50a is an elevated temperature 52a, for example, a high temperature 52c, of 1120 °F (one-thousand one-hundred twenty degrees Fahrenheit), and is at the compression ratio 161 of 10:1 in the graph 250d (see FIG. 5C). As shown in FIG. 5D, an original volume 254 of ambient air 30b (see FIG. 5B) is shown by the number 10, and the compressed volume 255 of compressed air 30f is shown by the number 1. The compressed volume 255 is 1/10 (one-tenth) of the original volume 254.

As shown in FIG. 5D, the compressor piston 174a in the compression chamber 172 has moved in the compressing direction 182 (see FIG. 5B) to a compression position 234, such as second compression position 234b, and shows that the compression of the compressed air 30f in the compression chamber 172 has reached 10, which corresponds to the compression ratio 161 of 10:1 in the graph 250d (see FIG. 5C). As shown in FIG. 5D, the compressed air 30f is compressed, and thereby heated, to an elevated temperature 52a (see also FIG. 16), such as a high temperature 52c (see also FIG. 16), of 1120 °F (one-thousand one-hundred twenty degrees Fahrenheit), and is at an elevated pressure 56a (see FIG. 16), such as a high pressure 56c (see FIG. 16). FIG. 5D shows the air intake openings 27 and the compressor valves 178a closed in the closed position 226. FIG. 5D further shows the rotation position 206, such as the alpha (α) rotation position 206a, of the compressor crankshaft 175a, at 325° (three-hundred twenty-five degrees).

As shown in FIG. 5D, the plenum 60, such as the vacuum-jacketed plenum tank 60b, has a high temperature 52c of 1120 °F (one-thousand one-hundred twenty degrees Fahrenheit) and a high pressure 56c of 369.2 psi (pounds per square inch) in the interior 82 of the plenum 60. FIG. 5D further shows the plenum inlet 62 and the plenum inlet valve 64 closed in the closed position 226. The plenum inlet valve 64 can be opened when the desired compression ratio 161 of 10:1 has been reached.

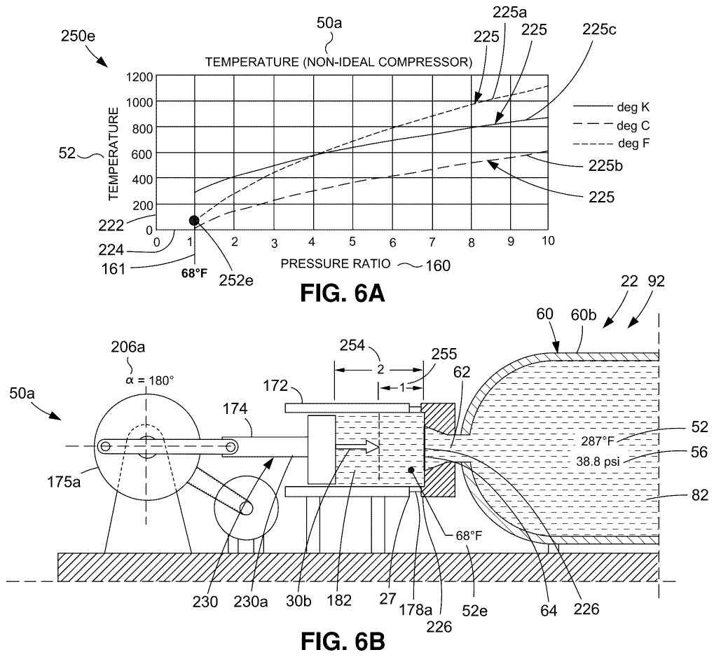

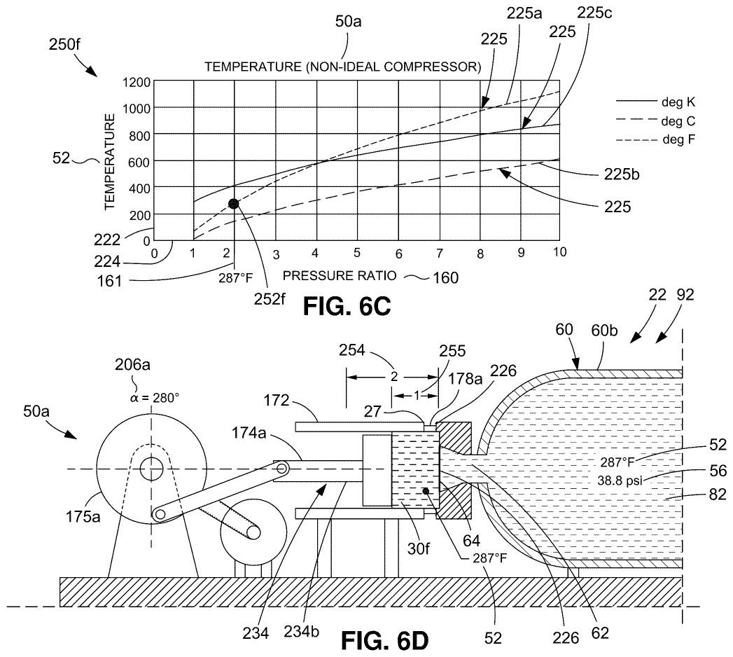

Now referring to FIG. 6A, FIG. 6A is an illustration of a graph 250e with the y-axis 222 of temperature 52 versus the x-axis 224 of pressure ratio 160 for a non-ideal compressor 50a. As shown in FIG. 6A, the graph 250e shows plots 225, including the degree Fahrenheit (deg F) plot 225a, the degree Celsius (deg C) plot 225b, and the degree Kelvin (deg K) plot 225c. FIG. 6A shows a plot point 252e at a temperature 52, such as ambient temperature 52e, of 68 °F (sixty-eight degrees Fahrenheit) and a compression ratio 161 of 1:1.

Now referring to FIG. 6B, FIG. 6B is an illustration of a side view of a non-ideal compressor 50a of a compressor assembly 22, such as a reciprocating compressor assembly 92, corresponding to the graph 250e in FIG. 6A, at ambient temperature 52e of 68 °F (sixty-eight degrees Fahrenheit) and the compression ratio 161 of 2:1 in the graph 250e (see FIG. 6A). As shown in FIG. 6B, the ambient air 30b in the compression chamber 172 of the non-ideal compressor 50a is at ambient temperature 52e of 68 °F (sixty-eight degrees Fahrenheit) and is at the compression ratio 161 of 1:1 in the graph 250e (see FIG. 6A). As shown in FIG. 6B, an original volume 254 of ambient air 30b is shown by the number 2, and a compressed volume 255 of compressed air 30f (see FIG. 6D) is shown by the number 1. The compressed volume 255 (see also FIG. 6D) is ½ (one-half) of the original volume 254.

As shown in FIG. 6B, the compression chamber 172 contains ambient air 30b at ambient temperature 52e of 68 °F (sixty-eight degrees Fahrenheit) and is at a constant pressure 56d (see FIG. 16). FIG. 6B shows the air intake openings 27 and the compressor valves 178a closed in the closed position 226. FIG. 6B further shows the rotation position 206, such as the alpha (α) rotation position 206a, of the compressor crankshaft 175a, at 180° (one-hundred eighty degrees). FIG. 6B further shows the compressor piston 174a in the compression chamber 172 in a retracted position 230, such as a fully retracted position 230a, that corresponds to the compression ratio 161 of 1:1 in the graph 250e (see FIG. 6A). The compressor piston 174a is configured to move in the compression chamber 172 toward the plenum inlet 62 of the plenum 60 in the compressing direction 182.

As shown in FIG. 6B, the plenum 60, such as the vacuum-jacketed plenum tank 60b, has a temperature 52 of 287 °F (two-hundred eighty-seven degrees Fahrenheit) (141 °C (one-hundred forty-one degrees Celsius)) and a pressure 56 of 38.8 psi (pounds per square inch) in the interior 82 of the plenum 60. FIG. 6B further shows the plenum inlet 62 and the plenum inlet valve 64 closed in the closed position 226.

Now referring to FIG. 6C, FIG. 6C is an illustration of a graph 250f with the y-axis 222 of temperature 52 versus the x-axis 224 of pressure ratio 160 for a non-ideal compressor 50a. As shown in FIG. 6C, the graph 250f shows plots 225, including the degree Fahrenheit (deg F) plot 225a, the degree Celsius (deg C) plot 225b, and the degree Kelvin (deg K) plot 225c. FIG. 6C shows a plot point 252f at a temperature 52 of 287 °F (two-hundred eighty-seven degrees Fahrenheit) (141 °C (one-hundred forty-one degrees Celsius), 414 K (four-hundred fourteen K)) and a compression ratio 161 of 2:1.

Now referring to FIG. 6D, FIG. 6D is an illustration of a side view of a non-ideal compressor 50a, of a compressor assembly 22, such as a reciprocating compressor assembly 92, corresponding to the graph 250f in FIG. 6C, where the compressed air 30f in the compression chamber 172 of the non-ideal compressor 50a is at the temperature 52 of 287 °F (two-hundred eighty-seven degrees Fahrenheit), and is at the compression ratio 161 of 2:1 in the graph 250f (see FIG. 6D). As shown in FIG. 6D, an original volume 254 of ambient air 30b (see FIG. 5B) is shown by the number 2, and the compressed volume 255 of compressed air 30f is shown by the number 1. The compressed volume 255 is ½ (one-half) of the original volume 254.

As shown in FIG. 6D, the compressor piston 174a in the compression chamber 172 has moved in the compressing direction 182 (see FIG. 6B) to a compression position 234, such as the second compression position 234b, and shows that the compression of the compressed air 30f in the compression chamber 172 has reached 2.0, which corresponds to the compression ratio 161 of 2:1 in the graph 250f (see FIG. 6C). As shown in FIG. 6D, the compressed air 30f is compressed, and thereby heated, to the temperature 52, of 287 °F (two-hundred eighty-seven degrees Fahrenheit). FIG. 6D shows the air intake openings 27 and the compressor valves 178a closed in the closed position 226. FIG. 6D further shows the rotation position 206, such as the alpha (α) rotation position 206a, of the compressor crankshaft 175a, at 280° (two-hundred eighty degrees).

As shown in FIG. 6D, the plenum 60, such as the vacuum-jacketed plenum tank 60b, has a temperature 52 of 287 °F (two-hundred eighty-seven degrees Fahrenheit) and a pressure 56 of 38.8 psi (pounds per square inch) in the interior 82 of the plenum 60. FIG. 6D further shows the plenum inlet 62 and the plenum inlet valve 64 closed in the closed position 226. The plenum inlet valve 64 can be opened when the desired compression ratio 161 of 2:1 has been reached.

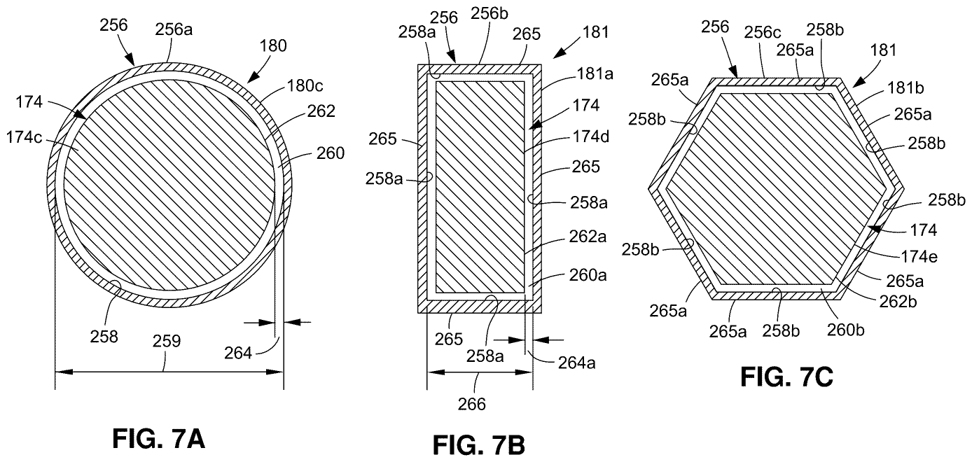

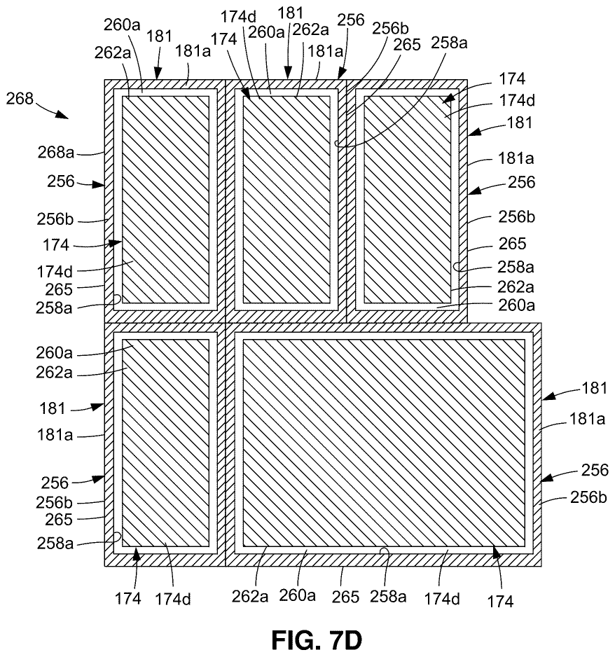

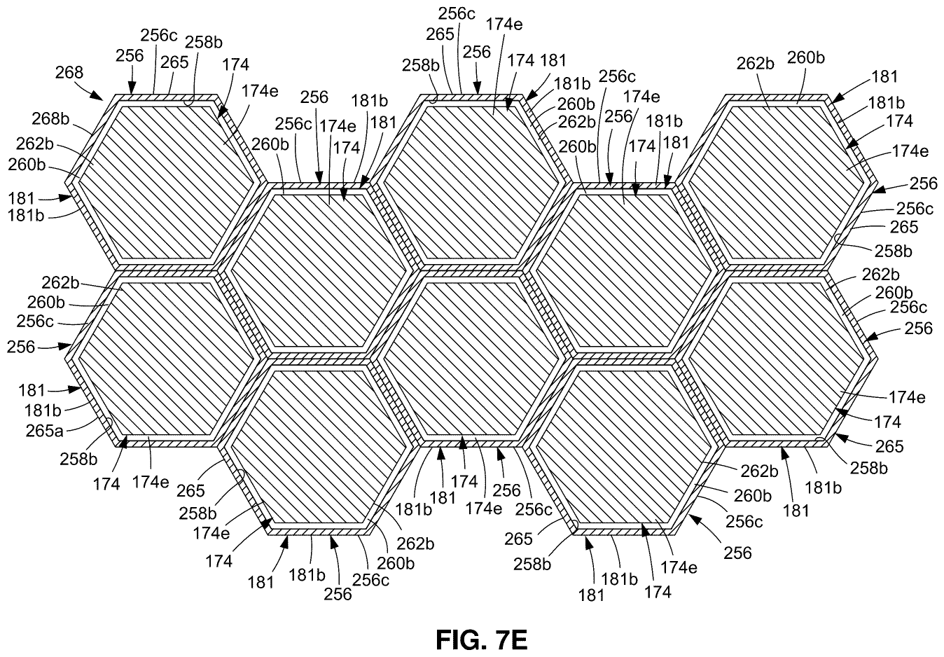

Now referring to FIG. 7A, FIG. 7A is an illustration of a cross-section of a cylinder 180, such as a cylindrical cylinder 180c, that may be used as a compression cylinder 180a (see FIG. 2A) and/or a decompression cylinder 180b (see FIG. 2A) in a version of a compressor assembly 22 (see FIG. 2A). The cylinder 180, such as the cylindrical cylinder 180c, houses the piston 174 (see FIG. 7A), such as a cylindrical piston 174c. As shown in FIG. 7A, the cylinder 180, such as the cylindrical cylinder 180c, has a cross-section shape 256 that is a circle shape 256a. As further shown in FIG. 7A, the cylinder 180, such as the cylindrical cylinder 180c, has an interior wall 258, and an inner diameter with an inner diameter length 259.

FIG. 7A further shows the piston 174, such as the cylindrical piston 174c, within the cylinder 180, such as the cylindrical cylinder 180c, and a gap 260 formed between an exterior 262 of the piston 174 and the interior wall 258. FIG. 7A further shows a width 264 of the gap 260.

For a piston 174 with an inner diameter length 259 that is large, a gap 260 that is small may be used. The gap 260 between the exterior 262 of the piston 174 and the interior wall 258 must be small enough so that the loss from choked flow, such as choked airflow, through the gap 260 is not too large. As used herein, “choked flow” means a condition in which an airflow through a narrowest constriction in the system, such as a gap, is sonic (Mach=1) and can only flow a certain velocity. Choked flow is a limiting condition where the airflow cannot increase with a further decrease in a downstream pressure environment for a fixed upstream pressure and temperature. If the area of the gap 260 is small in comparison to the area of the piston 174, the airflow is more efficient with minimal leakage.

Now referring to FIG. 7B, FIG. 7B is an illustration of a cross-section of a prism 181, such as a rectangular prism 181a, that may be used in a version of a compressor assembly 22 (see FIG. 16), where the cross-section shape 256 is a rectangle shape 256b. The shape of the prism 181 shown in FIG. 7B is the rectangular prism 181a. The prism 181, such as the rectangular prism 181a, houses the piston 174 (see FIG. 7B), such as a rectangular piston 174d (see FIG. 7B). As further shown in FIG. 7B, the prism 181, such as the rectangular prism 181a, has four (4) straight sides 265 with four (4) interior walls 258a. FIG. 7B further shows an inner width 266 between two (2) opposite interior walls 258a of two (2) opposite straight sides 265.

FIG. 7B further shows the piston 174, such as the rectangular piston 174d, within the prism 181, such as the rectangular prism 181a, and a gap 260a formed between an exterior 262a of the piston 174, such as the rectangular piston 174d, and the interior walls 258a of the rectangular prism 181a. FIG. 7B further shows a width 264a of the gap 260a.

Now referring to FIG. 7C, FIG. 7C is an illustration of another cross-section of a prism 181, such as a hexagonal prism 181b, that may be used in a version of a compressor assembly 22 (see FIG. 16), wherein the cross-section shape 256 is a hexagon shape 256c. The shape of the prism 181 shown in FIG. 7C is the hexagonal prism 181b. The prism 181, such as the hexagonal prism 181b, houses the piston 174 (see FIG. 7C), such as a hexagonal piston 174e (see FIG. 7C). As further shown in FIG. 7C, the prism 181, such as the hexagonal prism 181b, has six (6) straight sides 265a with six (6) interior walls 258b.

FIG. 7C further shows the piston 174, such as the hexagonal piston 174e, within the prism 181, such as the hexagonal prism 181b, and a gap 260b formed between an exterior 262b of the piston 174, such as the hexagonal piston 174e, and the interior walls 258b of the hexagonal prism 181b.

For faceted geometries, like the rectangular prism 181a and the hexagonal prism 181b, since the pressures are modest, e.g., approximately 50 psi (fifty pounds per square inch), that pressure could be taken by thick steel plates or stiffened structure made out of inexpensive steel plate. It may be easier to construct large smooth surfaces that way. In addition, the straight sides 265 of the rectangular prism 181a and the straight sides 265a of the hexagonal prism 181b do not need to be a solid plate, but may be a sandwich design for increased bending stiffness.

Now referring to FIG. 7D, FIG. 7D is an illustration of a cross-section of a prism arrangement 268, such as a tessellation prism arrangement 268a, of multiple prisms 181, such as rectangular prisms 181a. FIG. 7D shows five (5) rectangular prisms 181a, and each rectangular prism 181a has a cross-section shape 256 that is the rectangle shape 256b. Each prism 181, such as each rectangular prism 181a, houses the piston 174 (see FIG. 7D), such as the rectangular piston 174d (see FIG. 7D). As further shown in FIG. 7D, each prism 181, such as each rectangular prism 181a, has four (4) straight sides 265 with four (4) interior walls 258a. FIG. 7D further shows the piston 174, such as the rectangular piston 174d, within each prism 181, such as each rectangular prism 181a, and a gap 260a formed between an exterior 262a of each piston 174, such as each rectangular piston 174d, and the interior walls 258a of each rectangular prism 181a. The rectangular prisms 181a may use a same manifold 88 (see FIG. 16), or may use different manifolds 88, depending on the requirements of the air purification system 14 (see FIG. 16).

Now referring to FIG. 7E, FIG. 7E is an illustration of a cross-section of another prism arrangement 268, such as a nesting prism arrangement 268b, of multiple prisms 181, such as hexagonal prisms 181b. FIG. 7E shows ten (10) hexagonal prisms 181b, and each hexagonal prism 181b has a cross-section shape 256 that is the hexagon shape 256c. Each prism 181, such as each hexagonal prism 181b, houses the piston 174 (see FIG. 7E), such as the hexagonal piston 174e (see FIG. 7E). As further shown in FIG. 7E, each prism 181, such as each hexagonal prism 181b, has six (6) straight sides 265a with six (6) interior walls 258b. FIG. 7E further shows the piston 174, such as the hexagonal piston 174e, within each prism 181, such as each hexagonal prism 181b, and a gap 260b formed between an exterior 262b of each piston 174, such as each hexagonal piston 174e, and the interior walls 258b of each hexagonal prism 181b. The hexagonal prisms 181b may use the same manifold 88 (see FIG. 16), or may use different manifolds 88, depending on the requirements of the air purification system 14 (see FIG. 16).

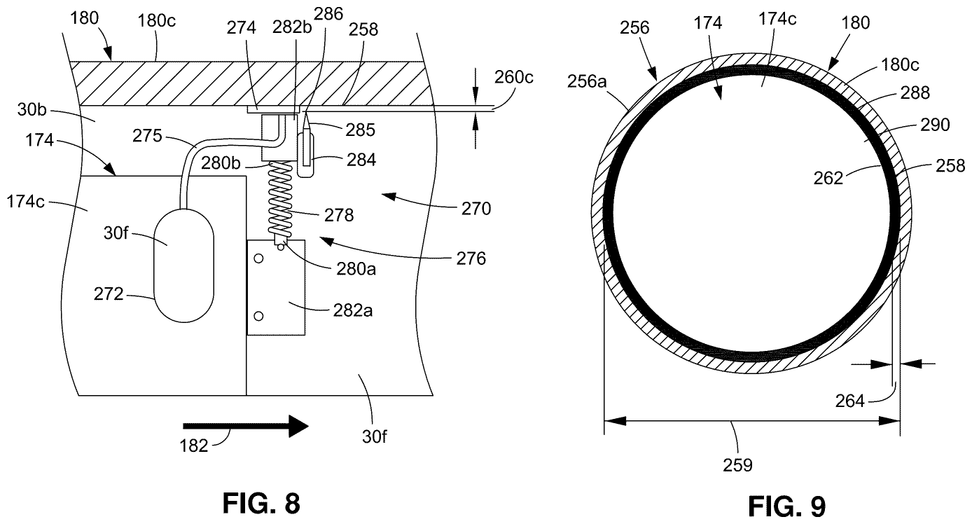

Now referring to FIG. 8, FIG. 8 is an illustration of a side view of a gap control assembly 270 for a cylinder 180, such as a cylindrical cylinder 180c, that may be used as a compression cylinder 180a (see FIG. 2A) and/or a decompression cylinder 180b (see FIG. 2A) in a version of a compressor assembly 22 (see FIGS. 2A, 16). In one version of the compressor assembly 22, the compression chamber 172 and the decompression chamber 210 each comprises the cylinder 180, such as the cylindrical cylinder 180c, having the gap control assembly 270, to control a size of a gap 260c (see FIG. 8) at the interior wall 258 (see FIG. 8) of the cylinder 180. In one version, as shown in FIG. 8, the cylinder 180 comprises the cylindrical cylinder 180c. In other versions, the prism 181, such as the rectangular prism 181a (see FIG. 7B), the hexagonal prism 181b (see FIG. 7C), or another geometrically shaped prism may be used.

As shown in FIG. 8, the gap control assembly 270 comprises a compressed air source 272 supplying compressed air 30f, and coupled to the piston 174, such as the cylindrical piston 174c, within the cylinder 180. As shown in FIG. 8, the gap control assembly 270 further comprises an air cushion element 274 coupled to the compressed air source 272, via an air line 275. As further shown in FIG. 8, the air cushion element 274 is coupled to the interior wall 258 of the cylinder 180. The compressed air source 272 provides compressed air 30f to the air cushion element 274, via the air line 275.

As shown in FIG. 8, the gap control assembly 270 further comprises a spring assembly 276 coupled between the piston 174 and the air cushion element 274. As further shown in FIG. 8, the spring assembly 276 comprises a spring 278 having a first end 280a and a second end 280b. As shown in FIG. 8, the first end 280a is coupled to a first attachment element 282a, which is attached to the piston 174, and the second end 280b is coupled to a second attachment element 282b, which is attached to the air cushion element 274.

As shown in FIG. 8, the gap control assembly 270 further comprises at least one blade element 284 coupled to the first attachment element 282a of the spring assembly 276. The blade element 284 has a blade tip 285 that is spaced a distance from the interior wall 258 of the cylinder 180 to form the gap 260c. The compressed air 30f from the compressed air source 272 works against the spring 278 of the spring assembly 276 to provide the gap 260c, and to control the size of the gap 260c between the interior wall 258 of the cylinder 180 and the blade tip 285 of the blade element 284. A ratio of a spring stiffness of the spring 278, to a compressed air pressure of the compressed air 30f, can be used to set or control the gap 260c size or magnitude, so the blade element 284 with the blade tip 285 of the gap control assembly 270 is positioned at a desired location 286 (see FIG. 8) relative to the gap 260c. The gap control assembly 270 provides a way to maintain a position of the blade element 284 to control the size of the gap 260c.

FIG. 8 further shows the piston 174 configured to move within the cylinder 180 in a compressing direction 182. FIG. 8 further shows ambient air 30b on one side of the spring assembly 276, and shows compressed air 30f on the other side of the spring assembly 276.

Now referring to FIG. 9, FIG. 9 is an illustration of a cross-section of a cylinder 180, such as a cylindrical cylinder 180c, that may be used as a compression cylinder 180a (see FIG. 2A) and/or a decompression cylinder 180b (see FIG. 2A) in a version of a compressor assembly 22 (see FIG. 2A), where the piston 174, such as the cylindrical piston 174c, has a piston ring 288 surrounding a circumference portion 290 of the piston 174. In one version of the compressor assembly 22, the compression chamber 172 and the decompression chamber 210 each comprises the cylinder 180, such as the cylindrical cylinder 180c, with the piston 174, such as the cylindrical piston 174c, within the cylinder 180, where the piston 174 has the piston ring 288. As shown in FIG. 9, the piston ring 288 fills the gap 260 (see FIG. 7A) between the exterior 262 of the piston 174, such as the cylindrical piston 174c, and the interior wall 258 of the cylinder 180, such as the cylindrical cylinder 180c. As shown in FIG. 9, the cylinder 180, such as the cylindrical cylinder 180c, has the cross-section shape 256 that is the circle shape 256a.

As further shown in FIG. 9, the cylinder 180, such as the cylindrical cylinder 180c, has the interior wall 258 and the inner diameter with the inner diameter length 259. FIG. 9 further shows the width 264 of the gap 260 that is now filled by the piston ring 288. For pistons 174 with smaller diameters, the piston ring 288 may be used. The piston ring 288 is preferably comprised of a material comprising one of, polytetrafluoroethylene (PTFE), nylon, rubber, fluoropolymer, a ceramic material, a thermoplastic material, or another suitable material that can withstand high temperatures 52c (see FIG. 16) and that can prevent leakage of air 30 (see FIG. 16) from the end of the piston 174.

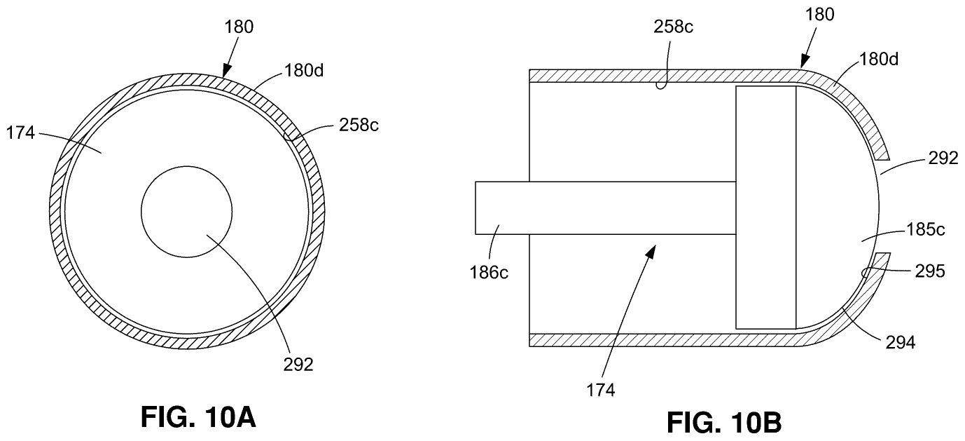

Now referring to FIGS. 10A-10B, FIG. 10A is an illustration of a cross-section of a piston 174 and a cylinder 180, such as a single opening cylinder 180d, having an opening 292, and FIG. 10B is an illustration of a side view of the piston 174 and the single opening cylinder 180d, of FIG. 10A. FIG. 10A shows the piston 174 within the cylinder 180, such as the single opening cylinder 180d. FIG. 10A further shows the interior wall 258c of the single opening cylinder 180d, and shows the opening 292 of the single opening cylinder 180d.

FIG. 10B shows the piston 174 within the cylinder 180, such as the single opening cylinder 180d. FIG. 10B further shows the interior wall 258c of the single opening cylinder 180d, and shows the opening 292 of the single opening cylinder 180d. FIG. 10B further shows the piston 174 with a curved piston head 185c coupled to a piston body 186c. As shown in FIG. 10B, the piston 174 has a piston end surface 294 on the curved piston head 185c that matches an interior end surface 295 of the single opening cylinder 180d.

As shown in FIG. 10B, the geometries of the piston 174 and the curved piston head 185c can be designed to be identical or closely matching, with little or no volume between them. This is because a plenum inlet valve 64 (see FIG. 2D) is designed to open so that the piston 174 can push all of the compressed air 30f in the cylinder 180, or the compression chamber 172, into the plenum 60.

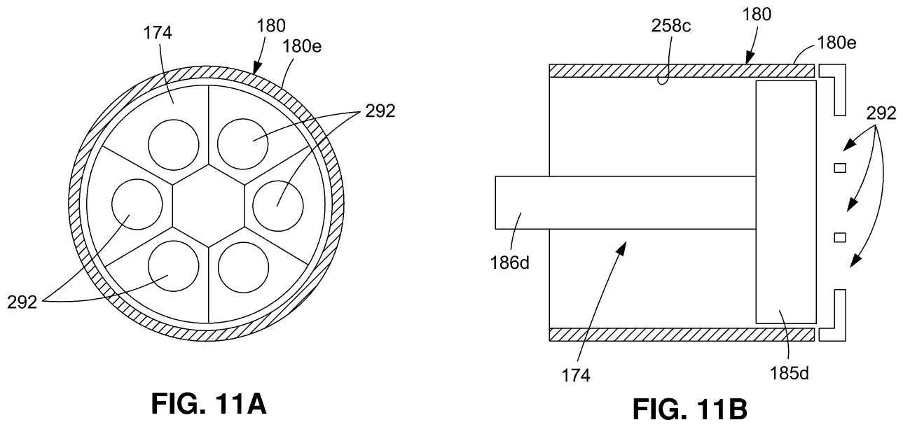

Now referring to FIG. 11A, FIG. 11A is an illustration of a cross-section of a piston 174 and a cylinder 180, such as a multiple opening cylinder 180e, having multiple openings 292, including seven (7) openings 292, and FIG. 11B is an illustration of a side view of the piston 174 and the multiple opening cylinder 180e, of FIG. 11A. FIG. 11A shows the piston 174 within the cylinder 180, such as the multiple opening cylinder 180e. FIG. 11A shows the interior wall 258c of the multiple opening cylinder 180e, and shows the multiple openings 292, including seven (7) openings 292, of the multiple opening cylinder 180e.

FIG. 11B shows the piston 174 within the cylinder 180, such as the multiple opening cylinder 180e. FIG. 11B further shows the interior wall 258c of the multiple opening cylinder 180e, and shows the multiple openings 292 of the multiple opening cylinder 180e. FIG. 11B further shows the piston 174 with a flat piston head 185d coupled to a piston body 186d. In this version of the piston 174 and the multiple opening cylinder 180e, it is advantageous to have multiple openings 292 per cylinder 180 to arrange for the shortest and most unobstructed path for the air 30, such as the compressed air 30f, to flow into the plenum 60.

Now referring to FIG. 12A, FIG. 12A is an illustration of a side view of a version of an air purification system 14 of the disclosure with the compressor assembly 22, in the form of a multi-nozzle turbine compressor assembly 96. FIG. 12A shows the multi-nozzle turbine compressor assembly 96 on the rooftop 145 of a building 12. The multi-nozzle turbine compressor assembly 96 is one variant of a turbine version.

As shown in FIG. 12A, the compressor assembly 22, such as the multi-nozzle turbine compressor assembly 96, comprises at least one compressor 50 comprising a multi-stage compressor 50b coupled to a gearbox 296, which in turn, is coupled to a motor 176, such as an electric motor 176a. FIG. 12A shows ambient air 30b at an ambient temperature 52e of 68 °F (sixty-eight degrees Fahrenheit) in the atmosphere 102, external to the compressor assembly 22. FIG. 12A shows the multi-stage compressor 50b in the compression and heating stage 171a.

As shown in FIG. 12A, the compressor assembly 22, such as the multi-nozzle turbine compressor assembly 96, further comprises at least one plenum 60 coupled to the multi-stage compressor 50b. As shown in FIG. 12A, the plenum 60 comprises a plenum tank 60a, such as a high-temperature plenum tank 60c. As shown in FIG. 12A, the plenum has the plenum inlet 62 and plenum inlet valve 64, and the plenum exit 66 and the plenum exit valve 68. FIG. 12A shows the plenum 60 in the high temperature dwell stage 171b. As shown in FIG. 12A, the plenum 60, such as the plenum tank 60a, has compressed air 30f at an elevated temperature 52a of 450 °F (four-hundred fifty degrees Fahrenheit) and a pressure 56 (see FIG. 16) of 68.4 psi (pounds per square inch) in the interior 82 of the plenum 60.

As shown in FIG. 12A, the compressor assembly 22, such as the multi-nozzle turbine compressor assembly 96, further comprises at least one decompressor 90 having a plurality of nozzles 298 coupled to the plenum exit 66. FIG. 12A shows the decompressor 90 in the decompression and cooling stage 171c, where purified air 30g received from the plenum 60 is decompressed into decompressed air 30h, that is cooled, and is expelled from the plurality of nozzles 298 into the ducts 106 inside the building 12, and into the interior airflow spaces 34 within the building 12.

The purified air 30g, such as decompressed air 30h, is cooled in the decompressor 90 to ambient temperature 52e of 68 °F (sixty-eight degrees Fahrenheit) and is at a constant pressure 56d (see FIG. 16), and the purified air 30g, such as the decompressed air 30h cooled to ambient temperature 52e of 68 °F (sixty-eight degrees Fahrenheit) is expelled into the ducts 106 inside the building 12, and into the interior airflow spaces 34 within the building 12. The purified air 30g, such as the decompressed air 30h, that is cooled, may also be expelled to secondary interior locations 152 (see FIG. 1A) in the building 12.

Now referring to FIG. 12B, FIG. 12B is an illustration of a side view of a version of an air purification system 14 of the disclosure with a compressor assembly 22, in the form of a generator turbine compressor assembly 98. FIG. 12B shows the generator turbine compressor assembly 98 on the rooftop 145 of a building 12. The generator turbine compressor assembly 98 is another variant of a turbine version.

As shown in FIG. 12B, the compressor assembly 22, such as the generator turbine compressor assembly 98, comprises at least one compressor 50 comprising a multi-stage compressor 50b coupled to a gearbox 296, which in turn, is coupled to a motor 176, such as an electric motor 176a. FIG. 12B shows ambient air 30b at an ambient temperature 52e of 68 °F (sixty-eight degrees Fahrenheit) in the atmosphere 102, external to the compressor assembly 22. FIG. 12B shows the multi-stage compressor 50b in the compression and heating stage 171a.

As further shown in FIG. 12B, the compressor assembly 22, such as the generator turbine compressor assembly 98, further comprises at least one plenum 60 coupled to the multi-stage compressor 50b. As shown in FIG. 12B, the plenum 60 comprises a plenum tank 60a, such as a high-temperature plenum tank 60c. As shown in FIG. 12B, the plenum has the plenum inlet 62 and plenum inlet valve 64, and the plenum exit 66 and the plenum exit valve 68. FIG. 12B further shows the plenum 60 in the high temperature dwell stage 171b. As shown in FIG. 12B, the plenum 60, such as the plenum tank 60a, has compressed air 30f at an elevated temperature 52a of 450 °F (four-hundred fifty degrees Fahrenheit) and a pressure 56 (see FIG. 16) of 68.4 psi (pounds per square inch) in the interior 82 of the plenum 60.

As further shown in FIG. 12B, the compressor assembly 22, such as the generator turbine compressor assembly 98, comprises at least one decompressor 90 having a nozzle 298a coupled to the plenum exit 66, and having a generator 212a coupled to the nozzle 298a. FIG. 12B shows the decompressor 90 in the decompression and cooling stage 171c, where purified air 30g received from the plenum 60 is decompressed into decompressed air 30h, that is cooled, and is expelled from the nozzle 298a into the ducts 106 inside the building 12, and into the interior airflow spaces 34 within the building 12.

The purified air 30g, such as decompressed air 30h, is cooled in the decompressor 90 to ambient temperature 52e of 68 °F (sixty-eight degrees Fahrenheit) and is at a constant pressure 56d (see FIG. 16), and the purified air 30g, such as the decompressed air 30h cooled to ambient temperature 52e of 68 °F (sixty-eight degrees Fahrenheit) is expelled into the ducts 106 inside the building 12, and into the interior airflow spaces 34 within the building 12. The purified air 30g, such as the decompressed air 30h, that is cooled, may also be expelled to secondary interior locations 152 (see FIG. 1A) in the building 12.

This version of the compressor assembly 22 in the form of the generator turbine compressor assembly 98 has the generator 212a to extract energy from the exiting purified air 30g to improve efficiency. The electrical power obtained helps drive the motor 176, such as the electric motor 176a.

Now referring to FIG. 13A, FIG. 13A is an illustration of a perspective view of a version of an air purification system 14 of the disclosure with a compressor assembly 22, in the form of a multi-cylinder compressor assembly 94. As shown in FIG. 13A, the compressor assembly 22, such as the multi-cylinder compressor assembly 94, comprises at least one compressor 50 having a plurality of intake cylinders 180f coupled to a plurality of intake pipes 300. As further shown in FIG. 13A, each intake cylinder 180f has an intake cylinder piston 174f coupled to an intake cylinder crankshaft 175c, via a connecting rod 194c. As further shown in FIG. 13A, the intake cylinder crankshaft 175c is coupled to an axle 302, such as a first axle 302a, inserted through a center hole 304a in each intake cylinder crankshaft 175c. As further shown in FIG. 13A, the first axle 302a is coupled to a drive train 306, and the drive train 306 is coupled to a motor 176. The compressor 50 is configured to compress, and to thereby heat, to the elevated temperature 52a (see FIG. 16), one or more of, the external air 30a (see FIG. 16), or ambient air 30b (see FIG. 16), and the recirculated air 30c (see FIG. 16), including contaminated air 30d (see FIG. 16), to obtain compressed air 30f (see FIG. 16).

As shown in FIG. 13A, the compressor assembly 22, such as the multi-cylinder compressor assembly 94, further comprises at least one plenum 60 comprising a plenum tank 60a with a plurality of plenum inlets 62 and a plurality of plenum exits 66. The plurality of plenum inlets 62 are coupled to the plurality of intake pipes 300. The plenum 60 is configured to maintain the compressed air 30f (see FIG. 16), that includes one or more of, the external air 30a, or ambient air 30b, and the recirculated air 30c, including contaminated air 30d, at the elevated temperature 52a for the effective dwell time 58a (see FIG. 16), to purify the compressed air 30f, that includes one or more of, the external air 30a, or ambient air 30b, and the recirculated air 30c, including contaminated air 30d, by deactivating or destroying the one or more biological agents 42 (see FIG. 16), and the one or more chemical agents 44 (see FIG. 16), present in the compressed air 30f, that includes one or more of, the external air 30a, or ambient air 30b, and the recirculated air 30c, including contaminated air 30d, to obtain the purified air 30g (see FIG. 16).

As further shown in FIG. 13A, the plenum 60, such as the plenum tank 60a, has a plenum length 308 and a plenum diameter 310. The plenum length 308 and the plenum diameter 310 may vary based on the size of the area for the compressor assembly 22 and the size of the building 12 or buildings 12 to be serviced with the compressor assembly 22.

As shown in FIG. 13A, the compressor assembly 22, such as the multi-cylinder compressor assembly 94, further comprises at least one decompressor 90 having a plurality of exit pipes 312 coupled to the plurality of plenum exits 66 of the plenum tank 60a, and having a plurality of exit cylinders 180g coupled to the plurality of exit pipes 312. As further shown in FIG. 13A, each exit cylinder 180g has an exit cylinder piston 174g coupled to an exit cylinder crankshaft 175d, via a connecting rod 194d. As further shown in FIG. 13A, the exit cylinder crankshaft 175d is coupled to an axle 302, such as a second axle 302b inserted through a center hole 304b in each exit cylinder crankshaft 175d. As further shown in FIG. 13A, the second axle 302b is coupled to the drive train 306, and the drive train 306 is coupled to the motor 176. The decompressor 90 is configured to decompress the purified air 30g, and also thereby cool the purified air 30g to the cooled temperature 52d (see FIG. 16), to obtain decompressed air 30h (see FIG. 16).

The plurality of intake cylinders 180f and the plurality of exit cylinders 180g each has a cross-section shape 256 (see FIGS. 7A, 13A) comprising one of, a circle shape 256a (see FIGS. 7A, 13A), a rectangle shape 256b (see FIG. 7B), a hexagon shape 256c (see FIG. 7C), or another suitable geometric shape. As shown in FIG. 13A, each intake cylinder 180f and each exit cylinder 180g has the cross-section shape 256 in the circle shape 256a, and comprise cylindrical cylinders 180c. The intake cylinders 180f and the exit cylinders 180g in the form of the cylindrical cylinders 180c may be roll-formed and made of thick steel or another suitable metal material.

The compressor assembly 22, such as the multi-cylinder compressor assembly 94, may further comprise valves 158 (see FIG. 13A) in the intake pipes 300 that open and close to let in or keep out compressed air 30f to the plenum 60, and valves 158 (see FIG. 13A) in the exit pipes 312 that open and close to let in or keep out to purified air 30g to the decompressor 90. The compressor assembly 22, such as the multi-cylinder compressor assembly 94, may further comprise and one or more manifolds 88 (see FIG. 16).

As shown in FIG. 13A, one or more bottom portions 314 of the compressor assembly 22, such as the multi-cylinder compressor assembly 94, may further be coupled to a frame support 316. The frame support 316 is designed to withstand an earthquake condition or event or other shaking condition or event, and provides support to the compressor assembly 22, such as the multi-cylinder compressor assembly 94, during an earthquake condition or event or other shaking condition or event. As shown in FIG. 13a, in one version, the frame support 316 comprises a rectangular frame support 316a that provides support to the compressor assembly 22, such as the multi-cylinder compressor assembly 94, at four places or corners of the rectangular frame support 316a. In another version, the frame support 316 may comprise a triangular frame support or another shape of frame support 316. A triangular frame support provides support to the compressor assembly 22, such as the multi-cylinder compressor assembly 94, at three places or points of the triangular frame support to ensure that the compressor assembly 22, such as the multi-cylinder compressor assembly 94, experiences only rigid-body movement with no warping.

Now referring to FIG. 13B, FIG. 13B is an illustration of a perspective view of the compressor assembly 22, in the form of a multi-cylinder compressor assembly 94, of FIG. 13A, with the plenum 60 removed. FIG. 13B shows the compressor 50 having the plurality of intake cylinders 180f coupled to the plurality of intake pipes 300, where each intake cylinder 180f has the intake cylinder piston 174f coupled to the intake cylinder crankshaft 175c, via the connecting rod 194c. As further shown in FIG. 13B, the intake cylinder crankshaft 175c is coupled to the axle 302, such as the first axle 302a, inserted through the center hole 304a in each intake cylinder crankshaft 175c. As further shown in FIG. 13B, the first axle 302a is coupled to the drive train 306, and the drive train 306 is coupled to the motor 176.

As shown in FIG. 13B, the compressor assembly 22, such as the multi-cylinder compressor assembly 94, further comprises the at least one decompressor 90 having the plurality of exit pipes 312 and having the plurality of exit cylinders 180g coupled to the plurality of exit pipes 312. As further shown in FIG. 13A, each exit cylinder 180g has the exit cylinder piston 174g coupled to the exit cylinder crankshaft 175d, via the connecting rod 194d. As further shown in FIG. 13A, the exit cylinder crankshaft 175d is coupled to the axle 302, such as the second axle 302b inserted through the center hole 304b in each exit cylinder crankshaft 175d. As further shown in FIG. 13A, the second axle 302b is coupled to the drive train 306, and the drive train 306 is coupled to the motor 176.

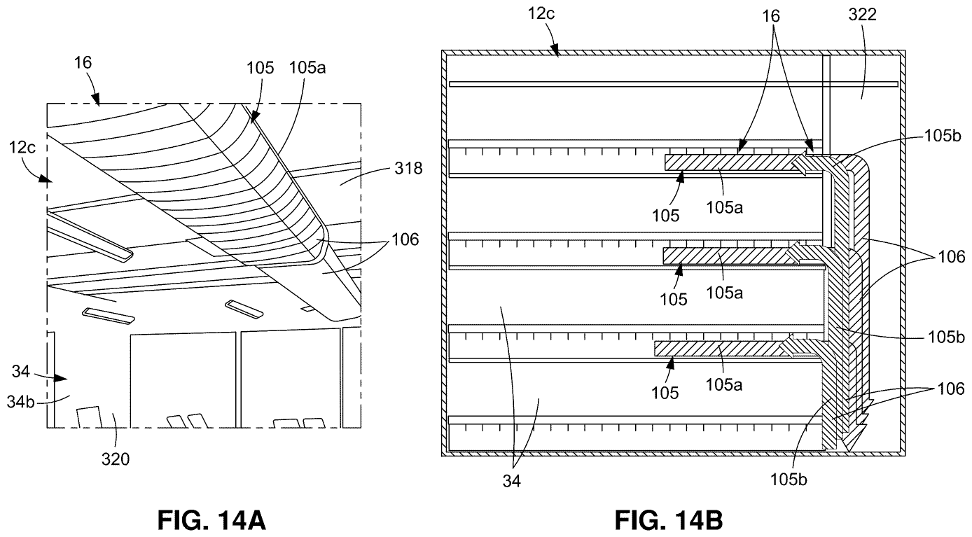

Now referring to FIG. 14A, FIG. 14A is an illustration of a perspective view of a duct network 105, such as an exposed interior duct network 105a, of the air management system 16 of the disclosure retrofitted in a retrofitted building 12c. As shown in FIG. 14A, the duct network 105, such as the exposed interior duct network 105a, of ducts 106 is suspended from a ceiling 318 above a workspace 320 in an interior airflow space 34, such as an office 34b. If there is no available space within the ceiling 318 or within the floor 140 to retrofit the duct network 105 of the air management system 16 in the retrofitted building 12c, the duct network 105, such as the exposed interior duct network 105a, of ducts 106 may be suspended from the ceiling 318.

Now referring to FIG. 14B, FIG. 14B is an illustration of a side view of the duct network 105, such as the exposed interior duct network 105a, of FIG. 14A, showing multiple exposed interior duct networks 105a with ducts 106 of the air management system 16 retrofitted in the interior airflow spaces 34 of retrofitted building 12c. FIG. 14B further shows external duct networks 105b of ducts 106 of the air management system 16 positioned external to the interior airflow spaces 34, such as in a stairway 322, and coupled to the exposed interior duct network 105a in the interior airflow spaces 34. The external duct networks 105b may also be positioned in elevator shafts, attached to the exterior of the retrofitted building 12c, or positioned in other areas of the retrofitted building 12c external to the interior airflow spaces 34. If internal space in the interior airflow spaces 34 does not allow for all of the ducts 106 needed for the air management system 16, the external duct networks 105b of ducts 106 may be coupled to the exposed interior duct network 105a, or may be coupled to other interior ducts within the ceiling 318 or the floor 140 of the retrofitted building 12c.

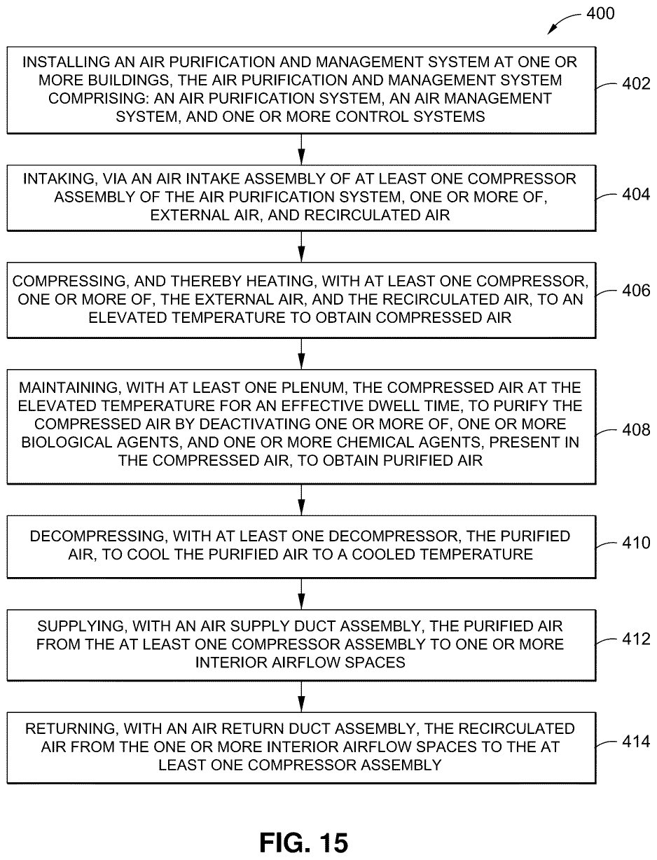

Now referring to FIG. 15, FIG. 15 is an illustration of a flow diagram of an exemplary version of a method 400 of the disclosure. In another version of the disclosure, there is provided the method 400 of purifying and managing air 30 (see FIG. 16), such as contaminated air 30d (see FIG. 16), at, and in, one or more buildings 12 (see FIGS. 1A, 16), such as one or more office buildings 12a (see FIGS. 1A, 16), and one or more commercial buildings 12b (see FIG. 16), and including both retrofitted buildings 12c (see FIG. 16) and new build buildings 12d (see FIG. 16), to provide a contamination mitigation 128 (see FIG. 16) to occupants 100 (see FIGS. 1A, 16), such as human occupants 100a (see FIGS. 1A, 16), of the one or more buildings 12. The blocks in FIG. 15 represent operations and/or portions thereof, or elements, and lines connecting the various blocks do not imply any particular order or dependency of the operations or portions thereof, or elements. FIG. 15 and the disclosure of the steps of the method 400 set forth herein should not be interpreted as necessarily determining a sequence in which the steps are to be performed. Rather, although one illustrative order is indicated, it is to be understood that the sequence of the steps may be modified when appropriate. Accordingly, certain operations may be performed in a different order or simultaneously.

As shown in FIG. 15, the method 400 comprises installing 402 the air purification and management system 10 (see FIG. 1A) at, and/or in, the one or more buildings 12. The air purification and management system 10 comprises an air purification system 14 (see FIG. 1A) comprising at least one compressor assembly 22 (see FIG. 1A). The at least one compressor assembly 22 (see FIG. 1A) comprises an air intake assembly 24 (see FIG. 1A) configured to take in one or more of, external air 30a (see FIG. 1A), or ambient air 30b (see FIG. 16), external to the one or more buildings 12. The external air 30a includes external air 30a contaminated with one or more of, one or more biological agents 42 (see FIGS. 1A, 16), and one or more chemical agents 44 (see FIGS. 1A, 16), and includes recirculated air 30c (see FIGS. 1A, 16) from one or more interior airflow spaces 34 (see FIG. 1A) in the one or more buildings 12. The recirculated air 30c comprises internal air 30e (see FIGS. 1A, 16) in the one or more interior airflow spaces 34. The internal air 30e includes internal air 30e, such as contaminated air 30d (see FIG. 16) contaminated with one or more of, the one or more biological agents 42, and the one or more chemical agents 44.

The air intake assembly 24 may comprise air intake openings 27 (see FIG. 16), such as at least one external air intake opening 28 (see FIG. 1A) configured to take in the external air 30a external to the one or more buildings 12, and/or may comprise at least two recirculated air intake openings 32 (see FIG. 1A), configured to take in recirculated air 30c (see FIG. 1A) from the one or more interior airflow spaces 34 (see FIG. 1A) in the one or more buildings 12.

The air purification system 14 further comprises at least one compressor 50 (see FIGS. 1A, 16), configured to compress, and thereby heat, to an elevated temperature 52 (see FIG. 16), one or more of, the external air 30a, and the recirculated air 30c. The air purification system 14 further comprises at least one plenum 60 (see FIGS. 1A, 16) coupled to the at least one compressor 50. The at least one plenum 60 has a plenum inlet 62 (see FIG. 1A) with a plenum inlet valve 64 (see FIG. 1A), and has a plenum exit 66 (see FIG. 1A) with a plenum exit valve 68 (see FIG. 1A). The at least one plenum 60 is configured to maintain one or more of, the external air 30a, and the recirculated air 30c, at the elevated temperature 52a for an effective dwell time 58a (see FIG. 16), to purify one or more of, the external air 30a, and the recirculated air 30c, by deactivating or destroying one or more of, the one or more biological agents 42, and the one or more chemical agents 44, present in one or more of, the external air 30a, and the recirculated air 30c, to obtain purified air 30g (see FIG. 16).

The air purification system 14 further comprises at least one decompressor 90 (see FIGS. 1A, 16) coupled to the at least one plenum 60. The at least one decompressor 90 is configured to decompress the purified air 30g, and also thereby cool the purified air 30g to a cooled temperature 52d (see FIG. 16). The air purification system 14 further comprises an air outlet assembly 26 (see FIGS. 1A, 16) coupled to the at least one decompressor 90. The air outlet assembly 26 is configured to direct the purified air 30g out of the at least one compressor assembly 22. The air outlet assembly 26 may comprise at least one air outlet exhaust opening 36 (see FIG. 1A) to exhaust the exhaust air 30i (see FIG. 16). The exhaust air 30i may comprise contaminated air 30d and/or purified air 30g. The air outlet assembly 26 may comprise at least one air outlet supply opening 38 (see FIG. 1A) to supply the purified air 30g.

The air purification and management system 10 further comprises an air management system 16 (see FIGS. 1A, 16) coupled to the air purification system 14 (see FIGS. 1A, 16). The air management system 16 comprises at least one external air intake duct 108 (see FIG. 1A) coupled to the air intake assembly 24, and in particular, coupled to the at least one external air intake opening 28 of the at least one compressor assembly 22. As shown in FIG. 1E, the air management system 16 comprises two external air intake ducts 108 comprising a first external air intake duct 108a and a second external air intake duct 108b.

The air management system 16 further comprises an air supply duct assembly 112 (see FIG. 1A) coupled between the air outlet assembly 26 of the at least one compressor assembly 22, and the one or more interior airflow spaces 34, to supply the purified air 30g from the at least one compressor assembly 22 to the one or more interior airflow spaces 34. In one version, as shown in FIG. 1A and discussed above, the air supply duct assembly 112 comprises a plurality of air supply ducts 114, including a plurality of low pressure air supply ducts 114a, and a plurality of moderate pressure air supply ducts 114b. Each of the plurality of air supply ducts 114 has an air pressure (AP) 115 (see FIG. 16) inside each air supply duct 114. Preferably, each of the plurality of low pressure air supply ducts 114a has a low air pressure (AP) 115a (see FIG. 16) in a range of from 0.002 psi (point zero zero two pound per square inch) to 0.05 psi (point zero five pound per square inch). Preferably, each of the plurality of moderate pressure air supply ducts 114b has a moderate air pressure (AP) 115b in a range of from 0.05 psi (point zero five pound per square inch) to 5.0 psi (five pounds per square inch).

The air management system 16 further comprises an air return duct assembly 116 (see FIG. 1A) coupled between the one or more interior airflow spaces 34, and the air intake assembly 24 of the at least one compressor assembly 22, to return the recirculated air 30c from the one or more interior airflow spaces 34 to the at least one compressor assembly 22. The recirculated air 30c comprises internal air 30e (see FIGS. 1A, 16) in the one or more interior airflow spaces 34 and may be mixed with purified air 30g from the compressor assembly 22. The internal air 30e includes, or may include, internal air 30e contaminated with one or more of, the one or more biological agents 42, and the one or more chemical agents 44.

In one version, as shown in FIG. 1A, the air return duct assembly 116 comprises a plurality of air return ducts 118, including a plurality of low pressure air return ducts 118a, and a plurality of moderate pressure air return ducts 118b. Each of the plurality of air return ducts 118 has an air pressure 115 (see FIG. 16) inside each air return duct 118. Preferably, each of the plurality of low pressure air return ducts 118a has a low air pressure 115a in a range of from 0.002 psi (point zero zero two pound per square inch) to 0.05 psi (point zero five pound per square inch). Preferably, each of the plurality of moderate pressure air return ducts 118b has a moderate air pressure 115b in a range of from 0.05 psi (zero point five pound per square inch) to 5.0 psi (five pounds per square inch). The air purification and management system 10 further comprises one or more control systems 18 (see FIGS. 1A, 16) configured to control the air purification system 14, and configured to control the air management system 16.

As shown in FIG. 15, the method 400 further comprises intaking 404, via the air intake assembly 24 of the at least one compressor assembly 22, one or more of, the external air 30a, and the recirculated air 30c. As shown in FIG. 15, the method 400 further comprises compressing 406, and thereby heating, with the at least one compressor assembly 22, one or more of, the external air 30a, and the recirculated air 30c, to an elevated temperature 52a (see FIG. 16).

As shown in FIG. 15, the method 400 further comprises maintaining 408, with the at least one plenum 60, one or more of, the external air 30a, and the recirculated air 30c, at the elevated temperature 52a for an effective dwell time 58a (see FIG. 16) and at an elevated pressure 56a (see FIG. 16) and an effective pressure 56b (see FIG. 16), to purify one or more of, the external air 30a, and the recirculated air 30c, by deactivating or destroying one or more of, the one or more biological agents 42, and the one or more chemical agents 44, present in one or more of, the external air 30a, and the recirculated air 30c, to obtain the purified air 30g (see FIG. 16).

As shown in FIG. 15, the method 400 further comprises decompressing 410, with the at least one decompressor 90, the purified air 30g, to cool the purified air 30g to a cooled temperature 52d (see FIG. 16). As shown in FIG. 15, the method 400 further comprises supplying 412, and transferring or moving, with the air supply duct assembly 112, the purified air 30g from the at least one compressor assembly 22 to the one or more interior airflow spaces 34.

As shown in FIG. 15, the method 400 further comprises returning 414, with the air return duct assembly 116, the recirculated air 30c from the one or more interior airflow spaces 34 to the at least one compressor assembly 22. The air purification and management system 10 provides the contamination mitigation 128 (see FIG. 16) to the occupants 100 (see FIG. 1A), such as human occupants 100a (see FIG. 1A), of the one or more buildings 12 from contamination by the one or more biological agents 42 (see FIG. 1A), and the one or more chemical agents 44.

The installing 402 (see FIG. 15) the air purification and management system 10 at, and/or in, the one or more buildings 12, may further comprise, in one version, installing and retrofitting the air purification and management system 10 in one or more retrofitted buildings 12c (see FIG. 16), or existing buildings, such as retrofitted office buildings, or existing office buildings, or retrofitted commercial buildings, or existing commercial buildings.

The installing 402 (see FIG. 15) the air purification and management system 10 at, and/or in, the one or more buildings 12, may further comprise, in another version, installing the air purification and management system 10 in one or more new build buildings 12d (see FIG. 16), thar are newly built or in the process of being built, such as new build office buildings, or new build commercial buildings.

The installing 402 the air purification and management system 10 at, and/or in, the one or more buildings 12, may further comprise installing the air purification and management system 10 further comprising a plurality of sensors 20 (see FIGS. 1A, 16). The plurality of sensors 20 comprise internal sensors 20a (see FIGS. 1A, 16) coupled to one or more of, the one or more interior airflow spaces 34 in the one or more buildings 12, and one or more secondary interior locations 152 (see FIG. 1B), such as non-airflow spaces, in the one or more buildings 12. The internal sensors 20a may also be coupled to other interior locations on, or in proximity to, the one or more buildings 12.

The plurality of sensors 20 further comprise external sensors 20b (see FIG. 1A) coupled to one or more of, the at least one external air intake duct 108, and one or more exterior portions 154 (see FIG. 1B) of the one or more buildings 12. The external sensors 20b may also be coupled to other exterior locations on, or in proximity to, the one or more buildings 12.

The installing 402 of the air purification and management system 10 at, and/or in, the one or more buildings 12, may further comprise installing the air purification and management system 10 further comprising the at least one compressor assembly 22 comprising a reciprocating compressor assembly 92 (see FIGS. 2A-2H). As shown in FIG. 2A, the reciprocating compressor assembly 92 further comprises at least one compressor 50 having a compression chamber 172 with a compressor piston 174a coupled to a compressor crankshaft 175a. As further shown in FIG. 2A, the compressor crankshaft 175a is coupled to an electric motor 176a. The at least one compressor 50 is configured to compress, and to thereby heat, to the elevated temperature 52a, one or more of, the external air 30a, and the recirculated air 30c, to obtain compressed air 30f (see FIG. 2A), contaminated with one or more of, the one or more biological agents 42 (see FIG. 2A), and/or the one or more chemical agents 44 (see FIG. 2A).

As further shown in FIG. 2A, the reciprocating compressor assembly 92 further comprises the at least one plenum 60 coupled to the at least one compressor 50. As shown in FIG. 2A, the at least one plenum 60 comprises a vacuum-jacketed plenum tank 60b with the plenum inlet 62 and the plenum exit 66. The plenum inlet 62 is coupled to the compression chamber 172. The at least one plenum 60 is configured to maintain the compressed air 30f, that includes one or more of, the external air 30a, and the recirculated air 30c, at the elevated temperature 52a (see FIG. 16) for the effective dwell time 58a (see FIG. 16), to purify the compressed air 30f, that includes one or more of, the external air 30a, and the recirculated air 30c, by deactivating or destroying the one or more biological agents 42, and the one or more chemical agents 44, present in the compressed air 30f, that includes one or more of, the external air 30a, and the recirculated air 30c, to obtain the purified air 30g.

As further shown in FIG. 2A, the reciprocating compressor assembly 92 further comprises the at least one decompressor 90 coupled to the plenum exit 66. As shown in FIG. 2A, the at least one decompressor 90 has a decompression chamber 210 with a decompressor piston 174b coupled to a decompressor crankshaft 175b, and the decompressor crankshaft 175b is coupled to a generator 212. The at least one decompressor 90 is configured to decompress the purified air 30g, and also thereby cool the purified air 30g to the cooled temperature 52d (see FIG. 16), to obtain decompressed air 30h.

The installing 402 of the air purification and management system 10 at, and/or in, the one or more buildings 12, may further comprise installing the air purification and management system 10 further comprising the at least one compressor assembly 22 comprising a multi-cylinder compressor assembly 94 (see FIG. 13A). As shown in FIG. 13A, the compressor assembly 22, such as the multi-cylinder compressor assembly 94, comprises at least one compressor 50 having a plurality of intake cylinders 180f coupled to a plurality of intake pipes 300. As further shown in FIG. 13A, each intake cylinder 180f has an intake cylinder piston 174f coupled to an intake cylinder crankshaft 175c, via a connecting rod 194c. As further shown in FIG. 13A, the intake cylinder crankshaft 175c is coupled to an axle 302, such as a first axle 302a, inserted through a center hole 304a in each intake cylinder crankshaft 175c. As further shown in FIG. 13A, the first axle 302a is coupled to a drive train 306, and the drive train 306 is coupled to a motor 176. The compressor 50 is configured to compress, and to thereby heat, to the elevated temperature 52a, one or more of, the external air 30a, or ambient air 30b, and the recirculated air 30c, including contaminated air 30d, to obtain compressed air 30f.

As further shown in FIG. 13A, the compressor assembly 22, such as the multi-cylinder compressor assembly 94, further comprises at least one plenum 60 comprising a plenum tank 60a with a plurality of plenum inlets 62 and a plurality of plenum exits 66. The plurality of plenum inlets 62 are coupled to the plurality of intake pipes 300. The plenum 60 is configured to maintain the compressed air 30f, that includes one or more of, the external air 30a, or ambient air 30b, and the recirculated air 30c, including contaminated air 30d, at the elevated temperature 52a for the effective dwell time 58a, to purify the compressed air 30f, that includes one or more of, the external air 30a, or ambient air 30b, and the recirculated air 30c, including contaminated air 30d, by deactivating or destroying the one or more biological agents 42, and the one or more chemical agents 44, present in the compressed air 30f, that includes one or more of, the external air 30a, or ambient air 30b, and the recirculated air 30c, including contaminated air 30d, to obtain the purified air 30g (see FIG. 16).

As further shown in FIG. 13A, the compressor assembly 22, such as the multi-cylinder compressor assembly 94, further comprises at least one decompressor 90 having a plurality of exit pipes 312 coupled to the plurality of plenum exits 66 of the plenum tank 60a, and having a plurality of exit cylinders 180g coupled to the plurality of exit pipes 312. As further shown in FIG. 13A, each exit cylinder 180g has an exit cylinder piston 174g coupled to an exit cylinder crankshaft 175d, via a connecting rod 194d. As further shown in FIG. 13A, the exit cylinder crankshaft 175d is coupled to an axle 302, such as a second axle 302b inserted through a center hole 304b in each exit cylinder crankshaft 175d. As further shown in FIG. 13A, the second axle 302b is coupled to the drive train 306, and the drive train 306 is coupled to the motor 176. The decompressor 90 is configured to decompress the purified air 30g, and also thereby cool the purified air 30g to the cooled temperature 52d (see FIG. 16), to obtain decompressed air 30h.

The plurality of intake cylinders 180f and the plurality of exit cylinders 180g each has a cross-section shape 256 (see FIGS. 7A, 13A) comprising one of, a circle shape 256a (see FIGS. 7A, 13A), a rectangle shape 256b (see FIG. 7B), a hexagon shape 256c (see FIG. 7C), or another suitable geometric shape.

The installing 402 of the air purification and management system 10 at, and/or in, the one or more buildings 12, may further comprise installing the air purification and management system 10 further comprising the at least one compressor assembly 22 comprising a multi-nozzle turbine compressor assembly 96 (see FIG. 12A). As shown in FIG. 12A, the multi-nozzle turbine compressor assembly 96 further comprises the at least one compressor 50 comprising a multi-stage compressor 50b coupled to a gearbox 296, which in turn, is coupled to a motor 176, such as an electric motor 176a. The at least one compressor 50 is configured to compress, and to thereby heat, to the elevated temperature 52a, one or more of, the external air 30a, such as ambient air 30b, and the recirculated air 30c, to obtain compressed air 30f.

As shown in FIG. 12A, the multi-nozzle turbine compressor assembly 96 further comprises at least one plenum 60 coupled to the multi-stage compressor 50b. As further shown in FIG. 12A, the at least one plenum 60 comprises a plenum tank 60a, such as a high-temperature plenum tank 60c, with the plenum inlet 62 and the plenum exit 66. The at least one plenum 60 is configured to maintain the compressed air 30f, that includes one or more of, the external air 30a, or ambient air 30b, and the recirculated air 30c, including contaminated air 30d, at the elevated temperature 52a for the effective dwell time 58a, to purify the compressed air 30f, that includes one or more of, the external air 30a, or ambient air 30b, and the recirculated air 30c, including contaminated air 30d, by deactivating or destroying one or more of, the one or more biological agents 42, and the one or more chemical agents 44, present in the compressed air 30f, that includes one or more of, the external air 30a, or ambient air 30b, and the recirculated air 30c, including contaminated air 30d, to obtain the purified air 30g.

As shown in FIG. 12A, the multi-nozzle turbine compressor assembly 96 further comprises at least one decompressor 90 having a plurality of nozzles 298 coupled to the plenum exit 66. The at least one decompressor 90 is configured to decompress the purified air 30g, and also thereby cool the purified air 30g to a cooled temperature 52d, to obtain decompressed air 30h.

The installing 402 of the air purification and management system 10 at, and/or in, the one or more buildings 12, may further comprise installing the air purification and management system 10 further comprising the at least one compressor assembly 22 comprising a generator turbine compressor assembly 98 (see FIG. 12B). As shown in FIG. 12B, the generator turbine compressor assembly 98 (see FIG. 12B) comprises the at least one compressor 50 comprising a multi-stage compressor 50b coupled to a gearbox 296, which in turn, is coupled to a motor 176, such as an electric motor 176a. The at least one compressor 50 is configured to compress, and to thereby heat, to the elevated temperature 52a, one or more of, the external air 30a, or ambient air 30b, and the recirculated air 30c, including contaminated air 30d, to obtain compressed air 30f.

As shown in FIG. 12B, the generator turbine compressor assembly 98 (see FIG. 12B) further comprises the at least one plenum 60 coupled to the multi-stage compressor 50b. As further shown in FIG. 12B, the at least one plenum 60 comprises a plenum tank 60a, such as a high-temperature plenum tank 60c, with the plenum inlet 62 and the plenum exit 66. The at least one plenum 60 is configured to maintain the compressed air 30f, that includes one or more of, the external air 30a, or ambient air 30b, and the recirculated air 30c, including contaminated air 30d, at the elevated temperature 52a for the effective dwell time 58a, to purify the compressed air 30f, that includes one or more of, the external air 30a, or ambient air 30b, and the recirculated air 30c, including contaminated air 30d, by deactivating or destroying one or more of, the one or more biological agents 42, and the one or more chemical agents 44, present in the compressed air 30f, that includes one or more of, the external air 30a, or ambient air 30b, and the recirculated air 30c, including contaminated air 30d, to obtain the purified air 30g.

As shown in FIG. 12B, the generator turbine compressor assembly 98 (see FIG. 12B) further comprises at least one decompressor 90 having a nozzle 298a coupled to the plenum exit 66, and having a generator 212a coupled to the nozzle 298a. The at least one decompressor 90 is configured to decompress the purified air 30g, and also thereby cool the purified air 30g to the cooled temperature 52d, to obtain decompressed air 30h.

The maintaining 408 (see FIG. 15), with the at least one plenum 60, the compressed air 30f at the elevated temperature 52a for the effective dwell time 58a, may further comprise preferably maintaining the elevated temperature 52a in a temperature range of from 140 °F (one-hundred forty degrees Fahrenheit) (60 °C (sixty degrees Celsius)) to 1500 °F (one thousand five hundred degrees Fahrenheit) (816 °C (eight hundred sixteen degrees Celsius)), and more preferably, in a temperature range of from 158 °F (one hundred fifty-eight degrees Fahrenheit) (70 °C (seventy degrees Celsius) to 600 °F (six hundred degrees Fahrenheit) (316 °C (three-hundred sixteen degrees Celsius)).

The compressed air 30f, that includes one or more of, the external air 30a, or ambient air 30b, and/or the recirculated air 30c, including contaminated air 30d, is preferably maintained at the elevated temperature 52a for the effective dwell time 58a comprising a range of 0.5 (zero point five) second to 30 (thirty) minutes, and more preferably, maintained at the elevated temperature 52a for the effective dwell time 58a comprising a range of 1 (one) second to 5 (five) minutes.

Maintaining 408 (see FIG. 15), with the at least one plenum 60, the compressed air 30f, that includes one or more of, the external air 30a, or ambient air 30b, and the recirculated air 30c, including contaminated air 30d, at the elevated temperature 52a for the effective dwell time 58a, and purifying the compressed air 30f, that includes one or more of, the external air 30a, or ambient air 30b, and the recirculated air 30c, including contaminated air 30d, by deactivating or destroying one or more of, the one or more biological agents 42, and the one or more chemical agents 44, may further comprise, deactivating or destroying the one or more biological agents 42 comprising one or more, airborne biological agents 42a (see FIG. 16), airborne pathogens, respiratory pathogens, airborne viruses, respiratory viruses, airborne microbes, respiratory microbes, airborne coronaviruses, including SARS-CoV-2 (severe acute respiratory syndrome coronavirus 2), COVID-19 (coronavirus disease 2019), MERS (Middle East respiratory syndrome), ebola virus, Zika virus, West Nile virus, Marburg virus, influenza virus, including, airborne influenza virus, influenza A virus, influenza B virus, influenza C virus, avian flu, and swine flu, smallpox virus, monkeypox virus,Streptococcus pneumoniae causing pneumonia, Mycobacterium tuberculosis causing tuberculosis,Bacillus anthracis causing anthrax, aspergillus fumigatus causing lung aspergillosis, black mold airborne spores, ricin mist, ricin powder, or another type of biological agent 42.

Maintaining 408 (see FIG. 15), with the at least one plenum 60, the compressed air 30f, that includes one or more of, the external air 30a, or ambient air 30b, and the recirculated air 30c, including contaminated air 30d, at the elevated temperature 52a for the effective dwell time 58a, and purifying the compressed air 30f, that includes one or more of, the external air 30a, or ambient air 30b, and the recirculated air 30c, including contaminated air 30d, by deactivating or destroying one or more of, the one or more biological agents 42, and the one or more chemical agents 44, may further comprise, deactivating or destroying the one or more chemical agents 44 comprising one or more, airborne chemical agents 44a (see FIG. 16), respiratory chemical agents, phenoxy herbicide, choking agents including chlorine, chloropicrin, diphosgene, and phosgene; blister agents including sulfur mustard, nitrogen mustard, lewisite, and phosgene oxime; blood agents including hydrogen cyanide, cyanogen chloride, and arsine; and nerve agents including tabun, sarin, soman, cyclosarin, and venomous agent X, or another type of chemical agent 44.

The method 400 may further comprise exhausting the exhaust air 30i and/or some, or an amount, of the purified air 30g out of the at least one air outlet exhaust opening 36 of the at least one compressor assembly 22, and into an atmosphere 102 (see FIG. 16) external to the one or more buildings 12.

The method 400 further comprises supplying 410 some, or an amount, of the purified air 30g, such as decompressed air 30h, out the at least two air outlet supply openings 38 of the at least one compressor assembly 22, through the air supply duct assembly 112 (see FIGS. 1A, 16), to the one or more interior airflow spaces 34 in the one or more buildings 12. The supplying 410 the purified air 30g, such as decompressed air 30h, and returning 412 the recirculated air 30c, may further comprise using the air supply duct assembly 112 and the air return duct assembly 116 (see FIGS. 1A, 16), to create controlled airflow patterns 125 (see FIG. 16) in the one or more interior airflow spaces 34 in an intermittent manner 126 (see FIG. 16), for a direct-path-to-return airflow 124 (see FIG. 16) in the one or more interior airflow spaces 34, to mitigate for the occupants 100 (see FIGS. 1A, 16) of the one or more buildings 12 against breathing contaminated air 30d contaminated by one or more of, the one or more biological agents 42, and the one or more chemical agents 44.

Now referring to FIG. 16, FIG. 16 is an illustration of a system block diagram of an exemplary version of the air purification and management system 10 of the disclosure for one or more buildings 12. The blocks in FIG. 16 represent elements, and lines connecting the various blocks do not imply any particular dependency of the elements. Furthermore, the connecting lines shown in the various Figures contained herein are intended to represent example functional relationships and/or physical couplings between the various elements, but it is noted that other alternative or additional functional relationships or physical connections may be present in versions disclosed herein. One or more of these blocks may be combined, divided, or combined and divided into different blocks when implemented in an illustrative example. Further, the illustrations of the air purification and management system 10 in FIG. 16 are not meant to imply physical or architectural limitations to the manner in which an illustrative example may be implemented. Other components in addition to, or in place of, the ones illustrated may be used. Some components may be unnecessary.

As shown in FIG. 16, the one or more buildings 12 may comprise one or more office buildings 12a, one or more commercial buildings 12b, or one or more other suitable buildings. As shown in FIG. 16, the one or more buildings 12, may comprise retrofitted buildings 12c that are already existing and already built, and that can be retrofitted with the air purification and management system 10 by installing the air purification and management system 10 at or in the retrofitted building 12c. The one or more buildings 12, such as the one or more office buildings 12a or the one or more commercial buildings 12b, may also comprise new build buildings 12d (see FIG. 16) that are newly built, or in the process of being built, and in which the air purification and management system 10 is installed or incorporated at, and/or in, the new build building 12d. As further shown in FIG. 16, the one or more buildings 12 contain interior (INT.) airflow spaces 34 in which the air management system 16 of the air purification system 14 is installed. As further shown in FIG. 16, the compressor assembly 22 of the air purification system 14 may be located in a basement 144 of a building 12, a rooftop 145 of a building 12, a ground area 146 of a building 12, an underground area 148 of a building 12, a structure 150 at or near a building 12, or another suitable area in or near a building 12.

As further shown in FIG. 16, the air purification and management system 10 is designed for contamination mitigation 128 for occupants 100, such as human occupants 100a, for example, contaminated (CONTAM.) occupants 100b, and/or animal occupants 100c, such as dogs and cats or other types of animals, of the one or more buildings 12 against contamination agents 40, such as one or more of, the one or more biological agents 42, such as airborne biological agents 42a, and the one or more chemical agents 44, such as airborne chemical agents 44a, that may be released in a pandemic 46, a biological weapon release 48, a chemical weapon release 49, or another type of release or event. The air purification and management system 10 is further designed for contamination mitigation 128 for occupants 100 of a building 12 against a contaminated occupant 100b in the building 12 that breathes a contamination agent 40, such as a biological agent 42, for example, an airborne biological agents 42a.

As shown in FIG. 16, the air purification and management system 10 comprises the air purification system 14, the air management system 16 coupled to the air purification system 14, and one or more control systems 18 configured to control, and controlling, the air purification system 14, and configured to control, and controlling, the air management system 16. The air purification and management system 10 may optionally comprise a plurality of sensors 20 (see FIGS. 1A-1D, 16), such as internal sensors 20a (see FIGS. 1A-1B, 1D) and external sensors 20b (see FIGS. 1A-1C).

As shown in FIG. 16, the air purification system 14 comprises at least one compressor assembly (ASSY.) 22, and in one version, may comprise a first compressor assembly (ASSY.) 22a and a second compressor assembly (ASSY.) 22b. As shown in FIG. 16, the compressor assembly 22 comprises the air intake assembly (ASSY.) 24, the compressor 50 having a compressor geometry 76, the plenum 60 having a plenum geometry (GEOM.) 78, the decompressor 90, and the air outlet assembly (ASSY.) 26. The plenum 60 may comprise a manifold 88 (see FIG. 16).

The air intake assembly 24 is configured to take in air 30 (see FIG. 16), such as external air 30a (see FIG. 16), for example, ambient air 30b (see FIG. 16), external to the one or more buildings 12. The air intake assembly 24 is further configured to take in recirculated (RECIRC.) air 30c (see FIG. 16), from one or more interior airflow spaces 34 in the one or more buildings 12. The external air 30a can include external air 30a in the form of contaminated air 30d (see FIG. 16) contaminated with one or more of, one or more contamination agents 40 (see also FIG. 16), such as one or more biological agents 42 (see also FIG. 16), and one or more chemical agents 44 (see also FIG. 16). The recirculated air 30c includes recirculated air 30c comprising internal air 30e (see FIG. 16), from within, or inside, the one or more interior airflow spaces 34. The internal air 30e can include contaminated air 30d. The plenum 60 is configured to purify, and does purify, the compressed air 30f to decontaminate the compressed air 30f, to obtain purified air 30g (see FIG. 16). The plenum 60 is further configured to repurify, and repurifies, the recirculated air 30c.

The air purification system 14 further comprises an air outlet assembly 26 (see FIGS. 1A, 16) comprising at least one air outlet exhaust opening 36 (see FIGS. 1A, 16), or multiple air outlet exhaust openings 36. As shown in FIG. 1A, the air purification system 14 comprises at least two air outlet supply openings 38, such as a first air outlet supply opening 38a, and a second air outlet supply opening 38b.

As shown in FIG. 1A, the external air 30a includes external air 30a in the form of contaminated air 30d contaminated with one or more of, one or more contamination agents 40 (see also FIG. 16), such as one or more biological agents 42 (see also FIG. 16), and one or more chemical agents 44 (see also FIG. 16). As shown in FIG. 1A, the recirculated air 30c includes recirculated air 30c comprising internal air 30e, from within, or inside, the one or more interior airflow spaces 34. The internal air 30e can include contaminated air 30d contaminated with one or more of, the one or more contamination agents 40 (see also FIG. 16), such as the one or more biological agents 42 (see also FIG. 16), and the one or more chemical agents 44 (see also FIG. 16), breathed by an occupant 100 (see also FIG. 16), such as a human occupant 100a (see also FIG. 16), for example, a contaminated occupant 100b (see also FIG. 16).

The compressor 50 is configured to compress, and compresses, and thereby heats, one or more of, the external air 30a, and the recirculated air 30c, to obtain compressed air 30f (see FIG. 16), such as compressed air 30f contaminated with one or more of, the one or more biological agents 42, and the one or more chemical agents 44. The decompressor 90 is configured to decompress, and decompresses, and thereby cools, the purified air 30g, to obtain decompressed (DECOMP.) air 30h. The air outlet assembly 26 is configured to exhaust or output exhaust (EXH.) air 30i (see FIG. 16). The exhaust air 30i may include purified air 30g and/or contaminated air 30d.

The compressor 50 compresses, and thereby heats, one or more of, the external air 30a, and the recirculated air 30c, to and at a temperature 52 (see FIG. 16), such as an elevated temperature 52a (see FIG. 16), and an effective temperature 52b (see FIG. 16), such as a high temperature (TEMP.) 52c (see FIG. 16). The decompressor 90 is configured to decompress, and decompresses, and thereby cools, the purified air 30g to a cooled temperature (TEMP.) 52d (see FIG. 16). The ambient air 30b in the atmosphere (ATM.) 102 (see FIG. 16), external to the compressor assembly 22, and/or external to the one or more buildings 12, is typically at an ambient temperature (TEMP.) 52e (see FIG. 16) of 68 °F (sixty-eight degrees Fahrenheit). When the temperature 52 of the air 30 is higher than the temperature 52 of the ambient air 30b and lower than the temperature 52 of the high temperature 52c, the temperature 52 comprises an intermediate temperature (TEMP.) 52f (see FIG. 16).

The compressor 50 compresses, and thereby heats, one or more of, the external air 30a, and the recirculated air 30c, to and at a pressure 56 (see FIG. 16), such as an elevated pressure 56a (see FIG. 16), and an effective pressure 56b (see FIG. 16), such as a high pressure 56c (see FIG. 16). The ambient air 30b is at a constant pressure 56d (see FIG. 16). When the pressure 56 of the air 30 is higher than the constant pressure 56d of the ambient air 30b and lower than the high pressure 56c, the pressure 56 comprises an intermediate pressure 56e (see FIG. 16).

The plenum 60 is configured to maintain, and maintains, the compressed air 30f, that includes one or more of, the external air 30a and the recirculated air 30c, at the elevated temperature 52a for a dwell time 58 (see FIG. 16), such as an effective (EFFECT.) dwell time 58a (see FIG. 16), to purify the compressed air 30f, by deactivating or destroying one or more of, the one or more biological agents 42, and the one or more chemical agents 44, present in the compressed air 30f, to obtain the purified air 30g. The compressed air 30f, that includes one or more of, the external air 30a, and/or the recirculated air 30c, is preferably maintained at a heat 54 (see FIG. 16) that is high and at the elevated temperature 52a, such as the high temperature 52c. In addition, elevated temperatures 52a (see FIG. 16), such as high temperatures 52c (see FIG. 16), may be achieved simply by changing the compression ratio (CR) 161 (see FIG. 16). The compression ratio 161 is easily adjustable in real time. A timing 72 (see FIG. 16) of the opening of the plenum inlet valve 64 (see FIG. 2A) of the plenum 60 is adjustable, to regulate the elevated temperature 52a and the elevated pressure 56a (see FIG. 16) inside the plenum 60.

As shown in FIG. 16, the compressor assembly 22 provides a purified air supply (AS) 70, or a clean air supply, of the purified air 30g, that is cooled in the decompressor 90, to the air management system 16. As shown in FIG. 16, the air management system 16 includes a duct network 105 comprised of a plurality of ducts 106 that may have one or more duct valves 107.

As shown in FIG. 16, the air management system 16 comprises one or more external air intake ducts 108 coupled to the compressor assembly 22. As shown in FIG. 16, the air management system 16 further comprises the air supply duct (ASD) assembly (ASSY.) 112 coupled between the air outlet assembly 26 of the compressor assembly 22 and the one or more interior airflow spaces 34, to supply the purified air 30g from the compressor assembly 22 to the one or more interior airflow spaces 34. In one version, as shown in FIG. 16, the air supply duct assembly 112 comprises a plurality of air supply ducts (ASD(S)) 114, including a plurality of low pressure (LP) air supply ducts (ASD(S)) 114a, and a plurality of moderate pressure (MP) air supply ducts (ASD(S)) 114b. Each of the plurality of air supply ducts 114 has an air pressure (PRESS.) (AP) 115 (see FIG. 16) inside each air supply duct 114. Preferably, in one version, each of the plurality of low pressure air supply ducts 114a has a low air pressure (AP) 115a (see FIG. 16) in a range of from 0.002 psi (point zero zero two pound per square inch) to 0.05 psi (point zero five pound per square inch). In other versions, the low air pressure 115a may be less than 0.002 psi or greater than 0.05 psi. Preferably, in one version, each of the plurality of moderate pressure air supply ducts 114b has a moderate air pressure (AP) 115b in a range of from 0.05 psi (point zero five pound per square inch) to 5.0 psi (five pounds per square inch). In other versions, the moderate air pressure 115b may be less than 0.05 psi or greater than 5.0 psi.

As shown in FIG. 16, the air management system 16 further comprises an air return duct (ARD) assembly (ASSY.) 116 coupled between the one or more interior airflow spaces 34 and the air intake assembly 24 of the compressor assembly 22, to return the recirculated air 30c from the one or more interior airflow spaces 34 to the compressor assembly 22 for purification or repurification.

In one version, as shown in FIG. 16, the air return duct assembly 116 comprises a plurality of air return ducts (ARD(S)) 118, including a plurality of low pressure (LP) air return ducts (ARD(S)) 118a, and a plurality of moderate pressure (MP) air return ducts (ARD(S)) 118b. Each of the plurality of air return ducts 118 has an air pressure 115 (see FIG. 16) inside each air return duct 118. Preferably, in one version, each of the plurality of low pressure air return ducts 118a has a low air pressure 115a in a range of from 0.002 psi (point zero zero two pound per square inch) to 0.05 psi (point zero five pound per square inch). In other versions, the low air pressure 115a may be less than 0.002 psi or greater than 0.05 psi. Preferably, in one version, each of the plurality of moderate pressure air return ducts 118b has a moderate air pressure 115b in a range of from 0.05 psi (point zero five pound per square inch) to 5.0 psi (five pounds per square inch). In other versions, the moderate air pressure 115b may be less than 0.05 psi or greater than 5.0 psi.

In one version, an interior 120a (see FIG. 1I) of one or more of the plurality of low pressure air return ducts 118a (see FIGS. 1I, 16), and an interior 120b (see FIG. 1J) of one or more of the plurality of moderate pressure air return ducts 118b (see FIGS. 1J, 16), contain one or more ultraviolet (UV) lights 122 (see FIGS. 1I, 1J, 16). The one or more ultraviolet lights 122 may be optionally used and located in the air return ducts 118 (see FIG. 16) to aid in deactivating or destroying one or more of the one or more biological agents 42 and/or one or more of the chemical agents 44. As an alternative to the ultraviolet lights 122, or in addition to the ultraviolet lights 122, the air return ducts 118 may have one or more grates with heated wires inside the air return ducts 118, or another suitable device or assembly to aid in deactivating or destroying one or more of the one or more biological agents 42 and/or one or more of the chemical agents 44.

As shown in FIG. 16, the air management system 16 may further comprise return air vents 165 in the ceiling to enforce the airflow (AF) 31 from the floor level 140b (see FIG. 1I) or the mid-height level, to the ceiling.

The airflow 31, such as surge airflow 31a (see FIG. 16), preferably has, in one version, a velocity 29 (see FIG. 16), or airflow speed, in a range of 3 ft/sec (three feet per second) to 5 ft/sec (five feet per second) in the interior airflow space 34. In other versions, the velocity 29, or airflow speed, of the surge airflow 31a is greater than 5 ft/sec (five feet per second). The surge airflow 31a (see FIG. 16) is surged into interior airflow spaces 34 with a controlled airflow (AF) pattern 125 (see FIG. 16) designed to move contaminated air 30d from a contaminated occupant 100b, or infected person, to the air return duct assembly 116 (see FIG. 16), without any other occupant 100 being in the path of the surge airflow 31a. After the occupant 100 has exited the interior airflow space 34, the surge airflow 31a is turned off, and the surge airflow 31a is returned to a normal airflow 31b (see FIG. 16) from the low pressure air supply ducts 114a (see FIG. 16).

The air supply duct assembly 112 and the air return duct assembly 116 create controlled airflow (AF) patterns 125 (see FIG. 16) in the interior airflow spaces 34 in an intermittent manner 126 (see FIG. 16), to mitigate the occupants 100 in the building 12 from breathing contaminated air 30d contaminated by one or more of, the one or more biological agents 42, and the one or more chemical agents 44. The intermittent manner 126 means the purified air 30g is introduced, or flows, into the interior airflow spaces 34 only when the occupant 100, such as the human occupant 100a, is present in the interior airflow space 34.

As shown in FIG. 16, the interior airflow spaces 34 are designed for a direct-path-to-return airflow (AF) 124. The direct-path-to-return airflow 124 is an airflow field designed and achieved for which there is a very low probability of an occupant 100, such as a human occupant 100a, of the building 12, breathing air 30, such as contaminated air 30d, that has already been breathed by another occupant 100, such as another human occupant 100a, including a contaminated occupant 100b, nearby in the interior airflow space 34, and in turn, any other occupant 100, such as any other human occupant 100a, including a contaminated occupant 100b, in the building 12 in any other interior airflow space 34.

As shown in FIG. 16, the air purification and management system 10 includes the control system (SYS.) (CS) 18 comprising a sensor control network 130 with one or more controllers 132, one or more power supplies 134 to power the control system 18, one or more software programs (PROG. (S)) 135, and one or more computers 136 configured to run the one or more software programs 135. As shown in FIG. 16, in one version, the control system 18 is a central control system (CS) 18a. The central control system 18a is coupled to the compressor assembly 22, the air supply duct assembly 112, and the air return duct assembly 116, via connector elements 137 (see FIG. 1A), such as wired connectors or wireless connectors. In another version, the control system 18 comprises a distributed control system (CS) 18b, in which the sensor control network 130, one or more controllers 132, one or more power supplies 134, and one or more computers 136 with one or more software programs (PROG. (S)) 135, are distributed at each interior airflow space 34 and/or secondary interior location 152, or at several interior airflow spaces 34 and/or secondary interior locations 152 on the same floor 140 (see FIG. 1B) or on adjacent floors 140.

Disclosed versions of the air purification and management system 10 (see FIG. 1A) and method 400 (see FIG. 15) provide for an improved air purification and management system 10 and method 400 for buildings 12, such as office buildings 12a (see FIG. 16) and commercial buildings 12b (see FIG. 16), that provide effective temperatures 52b (see FIG. 16), such as effective heating temperatures, and effective dwell times 58 (see FIG. 16), such as effective dwell times 58a (see FIG. 16), to destroy numerous types of biological agents 42 (see FIG. 16) and chemical agents 44 (see FIG. 16), that provide effective temperatures 52b, such as effective heating temperatures above 392 °F (three-hundred ninety-two degrees Fahrenheit) (200 °C (two-hundred degrees Celsius)), that provide an easily adjustable maximum temperature, that enable the destruction of any biological agent 42 or chemical agent 44 by increasing effective temperatures 52b, such as effective heating temperatures, to a maximum, that purify the air 30 (see FIG. 16) to near zero risk, that do not damage or harm occupants 100 (see FIG. 16), such as human occupants 100a (see FIG. 16) and/or animal occupants 100c (see FIG. 16) of a building 12, that provide contamination mitigation 128 (see FIG. 16) for occupants 100 of buildings 12 against contamination by biological agents 42 and chemical agents 44, that do not require any filters to change or maintain, and that provide advantages over known systems and methods.

Many modifications and other versions of the disclosure will come to mind to one skilled in the art to which this disclosure pertains having the benefit of the teachings presented in the foregoing descriptions and the associated drawings. The versions described herein are meant to be illustrative and are not intended to be limiting or exhaustive. Although specific terms are employed herein, they are used in a generic and descriptive sense only and not for purposes of limitation. Functionally equivalent methods and apparatuses within the scope of the disclosure, in addition to those enumerated herein, are possible from the foregoing descriptions. Such modifications and variations are intended to fall within the scope of the appended claims. The present disclosure is to be limited only by the terms of the appended claims, along with the full scope of equivalents to which such claims are entitled.

1. An air purification and management system for one or more buildings, the air purification and management system comprising:
an air purification system comprising:
at least one compressor assembly comprising:
an air intake assembly configured to take in one or more of:
external air external to the one or more buildings, the external air including external air contaminated with one or more of, one or more biological agents, and one or more chemical agents, and
recirculated air from one or more interior airflow spaces in the one or more buildings, the recirculated air comprising internal air in the one or more interior airflow spaces, the internal air including internal air contaminated with one or more of, the one or more biological agents, and the one or more chemical agents;
at least one compressor configured to compress, and thereby heat, to an elevated temperature, one or more of, the external air, and the recirculated air, to obtain compressed air, contaminated with one or more of, the one or more biological agents, and the one or more chemical agents;
at least one plenum coupled to the at least one compressor, the at least one plenum having a plenum inlet with a plenum inlet valve, and having a plenum exit with a plenum exit valve, the at least one plenum configured to maintain the compressed air at the elevated temperature for an effective dwell time, to purify the compressed air by deactivating one or more of, the one or more biological agents, and the one or more chemical agents, present in the compressed air, to obtain purified air;
at least one decompressor coupled to the at least one plenum, the at least one decompressor configured to decompress the purified air, and thereby cool the purified air to a cooled temperature; and
an air outlet assembly coupled to the at least one decompressor, the air outlet assembly configured to direct the purified air out of the at least one compressor assembly;
an air management system coupled to the air purification system, the air management system comprising:
at least one external air intake duct coupled to the air intake assembly of the at least one compressor assembly;
an air supply duct assembly coupled between the air outlet assembly of the at least one compressor assembly and the one or more interior airflow spaces, to supply the purified air from the at least one compressor assembly to the one or more interior airflow spaces; and
an air return duct assembly coupled between the one or more interior airflow spaces and the air intake assembly of the at least one compressor assembly, to return the recirculated air from the one or more interior airflow spaces to the at least one compressor assembly; and
one or more control systems configured to control the air purification system, and configured to control the air management system,
wherein the air purification and management system provides a contamination mitigation for occupants of the one or more buildings against contamination by one or more of, the one or more biological agents, and the one or more chemical agents.

2. The air purification and management system of claim 1, further comprising a plurality of sensors comprising:
internal sensors coupled to one or more of, the one or more interior airflow spaces in the one or more buildings, and one or more secondary interior locations in the one or more buildings; and
external sensors coupled to one or more of, the at least one external air intake duct, and one or more exterior portions of the one or more buildings.

3. The air purification and management system of claim 1, wherein the at least one compressor assembly is located:
in a basement of one or more of the one or more buildings;
on a rooftop of one or more of the one or more buildings;
on a ground area adjacent to one or more of the one or more buildings;
in an underground area adjacent to one or more of the one or more buildings; or
in a structure in proximity to one or more of the one or more buildings.

4. The air purification and management system of claim 1, wherein the at least one compressor assembly comprises a reciprocating compressor assembly further comprising:
the at least one compressor having a compression chamber with a compressor piston coupled to a compressor crankshaft, and the compressor crankshaft coupled to an electric motor;
the at least one plenum comprising a vacuum-jacketed plenum tank with the plenum inlet coupled to the compression chamber; and
the at least one decompressor coupled to the plenum exit, and the at least one decompressor having a decompression chamber with a decompressor piston coupled to a decompressor crankshaft, and the decompressor crankshaft coupled to a generator.

5. The air purification and management system of claim 1, wherein the at least one plenum comprises a plurality of baffles positioned within an interior of the at least one plenum.

6. The air purification and management system of claim 1, wherein the at least one plenum comprises a heat exchanger with internal airflow passages.

7. The air purification and management system of claim 1, wherein the at least one plenum comprises a coil.

8. The air purification and management system of claim 1, wherein the at least one compressor assembly comprises a generator turbine compressor assembly further comprising:
the at least one compressor comprising a multi-stage compressor coupled to a gearbox, which is coupled to an electric motor;
the at least one plenum coupled to the multi-stage compressor, the at least one plenum comprising a plenum tank; and
the at least one decompressor having a nozzle coupled to the plenum exit, and having a generator coupled to the nozzle.

9. The air purification and management system of claim 1, wherein one or more of:
the one or more biological agents comprise one or more, airborne biological agents, airborne pathogens, respiratory pathogens, airborne viruses, respiratory viruses, airborne microbes, respiratory microbes, airborne coronaviruses, including SARS-CoV-2 (severe acute respiratory syndrome coronavirus 2), COVID-19 (coronavirus disease 2019), MERS (Middle East respiratory syndrome), ebola virus, Zika virus, West Nile virus, Marburg virus, influenza virus, including, airborne influenza virus, influenza A virus, influenza B virus, influenza C virus, avian flu, and swine flu, smallpox virus, monkeypox virus,Streptococcus pneumoniae causing pneumonia,Mycobacterium tuberculosis causing tuberculosis, Bacillus anthracis causing anthrax, aspergillus fumigatus causing lung aspergillosis, pneumonic plague, black mold airborne spores, ricin mist, and ricin powder; and
one or more chemical agents comprise one or more, airborne chemical agents, respiratory chemical agents, phenoxy herbicide, choking agents including chlorine, chloropicrin, diphosgene, and phosgene; blister agents including sulfur mustard, nitrogen mustard, lewisite, and phosgene oxime; blood agents including hydrogen cyanide, cyanogen chloride, and arsine; and nerve agents including tabun, sarin, soman, cyclosarin, and venomous agent X.

10. An air purification system for one or more buildings, the air purification system comprising:
at least one compressor assembly comprising:
an air intake assembly comprising at least one external air intake opening, and at least two recirculated air intake openings, the air intake assembly configured to take in one or more of:
external air external to the one or more buildings, the external air including external air contaminated with one or more of, one or more biological agents, and one or more chemical agents, and
recirculated air from one or more interior airflow spaces in the one or more buildings, the recirculated air comprising internal air in the one or more interior airflow spaces, the internal air including internal air contaminated with one or more of, the one or more biological agents, and the one or more chemical agents;
at least one compressor configured to compress, and thereby heat, to an elevated temperature, one or more of, the external air, and the recirculated air, to obtain compressed air, contaminated with one or more of, the one or more biological agents, and the one or more chemical agents;
at least one plenum coupled to the at least one compressor, the at least one plenum having a plenum inlet with a plenum inlet valve, and having a plenum exit with a plenum exit valve, the at least one plenum configured to maintain the compressed air at the elevated temperature for an effective dwell time, to purify the compressed air by deactivating one or more of, the one or more biological agents, and the one or more chemical agents, present in the compressed air, to obtain purified air;
at least one decompressor coupled to the at least one plenum, the at least one decompressor configured to decompress the purified air, and thereby cool the purified air to a cooled temperature; and
an air outlet assembly coupled to the at least one decompressor, and comprising at least one air outlet exhaust opening, and at least one air outlet supply opening, the air outlet assembly configured to direct the purified air out of the at least one compressor assembly; and
a control system configured to control the air purification system,
wherein the air purification system is configured for use with an air management system, to supply the purified air to the one or more interior airflow spaces in the one or more buildings, and to receive the recirculated air from the one or more interior airflow spaces, to provide a contamination mitigation for occupants of the one or more buildings against contamination by one or more of, the one or more biological agents, and the one or more chemical agents.

11. The air purification system of claim 10, wherein the at least one compressor assembly comprises a reciprocating compressor assembly further comprising:
the at least one compressor having a compression chamber with a compressor piston coupled to a compressor crankshaft, and the compressor crankshaft coupled to an electric motor;
the at least one plenum comprising a vacuum-jacketed plenum tank with a plenum inlet and a plenum exit, and the plenum inlet coupled to the compression chamber; and
the at least one decompressor coupled to the plenum exit, and the at least one decompressor having a decompression chamber with a decompressor piston coupled to a decompressor crankshaft, and the decompressor crankshaft coupled to a generator.

12. The air purification system of claim 10, wherein the at least one compressor assembly comprises a multi-cylinder compressor assembly further comprising:
the at least one compressor having a plurality of intake cylinders coupled to a plurality of intake pipes, each intake cylinder having an intake cylinder piston coupled to an intake cylinder crankshaft, which is coupled to a first axle, the first axle coupled to a drive train, and the drive train coupled to a motor;
the at least one plenum comprising a plenum tank with a plurality of plenum inlets and a plurality of plenum exits, the plurality of plenum inlets coupled to the plurality of intake pipes; and
the at least one decompressor having a plurality of exit pipes coupled to the plurality of plenum exits of the plenum tank, and having a plurality of exit cylinders coupled to the plurality of exit pipes, each exit cylinder having an exit cylinder piston coupled to an exit cylinder crankshaft, which is coupled to a second axle, the second axle coupled to the drive train, and the drive train coupled to the motor.

13. The air purification system of claim 10, wherein the at least one compressor assembly comprises a generator turbine compressor assembly further comprising:
the at least one compressor comprising a multi-stage compressor coupled to a gearbox, which is coupled to an electric motor;
the at least one plenum coupled to the multi-stage compressor, the at least one plenum comprising a plenum tank with the plenum inlet and the plenum exit; and
the at least one decompressor having a nozzle coupled to the plenum exit, and having a generator coupled to the nozzle.

14. The air purification system of claim 10, wherein one or more of:
the one or more biological agents comprise one or more, airborne biological agents, airborne pathogens, respiratory pathogens, airborne viruses, respiratory viruses, airborne microbes, respiratory microbes, airborne coronaviruses, including SARS-CoV-2 (severe acute respiratory syndrome coronavirus 2), COVID-19 (coronavirus disease 2019), MERS (Middle East respiratory syndrome), ebola virus, Zika virus, West Nile virus, Marburg virus, influenza virus, including, airborne influenza virus, influenza A virus, influenza B virus, influenza C virus, avian flu, and swine flu, smallpox virus, monkeypox virus,Streptococcus pneumoniae causing pneumonia,Mycobacterium tuberculosis causing tuberculosis, Bacillus anthracis causing anthrax, aspergillus fumigatus causing lung aspergillosis, pneumonic plague, black mold airborne spores, ricin mist, and ricin powder; and
the one or more chemical agents comprise one or more, airborne chemical agents, respiratory chemical agents, phenoxy herbicide, choking agents including chlorine, chloropicrin, diphosgene, and phosgene; blister agents including sulfur mustard, nitrogen mustard, lewisite, and phosgene oxime; blood agents including hydrogen cyanide, cyanogen chloride, and arsine; and nerve agents including tabun, sarin, soman, cyclosarin, and venomous agent X.

15. A method of purifying and managing air at one or more buildings, to provide a contamination mitigation to occupants of the one or more buildings, the method comprising:
installing an air purification and management system at the one or more buildings, the air purification and management system comprising:
an air purification system comprising:
at least one compressor assembly comprising:
an air intake assembly configured to take in one or more of:
external air external to the one or more buildings, the external air including external air contaminated with one or more of, one or more biological agents, and one or more chemical agents, and
recirculated air from one or more interior airflow spaces in the one or more buildings, the recirculated air comprising internal air in the one or more interior airflow spaces, the internal air including internal air contaminated with one or more of, the one or more biological agents, and the one or more chemical agents;
at least one compressor;
at least one plenum coupled to the at least one compressor, the at least one plenum having a plenum inlet with a plenum inlet valve, and having a plenum exit with a plenum exit valve;
at least one decompressor coupled to the at least one plenum; and
an air outlet assembly coupled to the at least one decompressor, the air outlet assembly configured to direct purified air out of the at least one compressor assembly;
an air management system coupled to the air purification system, the air management system comprising:
at least one external air intake duct coupled to the air intake assembly of the at least one compressor assembly;
an air supply duct assembly coupled between the air outlet assembly of the at least one compressor assembly and the one or more interior airflow spaces;
an air return duct assembly coupled between the one or more interior airflow spaces and the air intake assembly of the at least one compressor assembly; and
one or more control systems configured to control the air purification system, and configured to control the air management system,
intaking, via the air intake assembly of the at least one compressor assembly, one or more of, the external air, and the recirculated air;
compressing, and thereby heating, with the at least one compressor, one or more of, the external air, and the recirculated air, to an elevated temperature, to obtain compressed air, contaminated with one or more of, the one or more biological agents, and the one or more chemical agents;
maintaining, with the at least one plenum, the compressed air at the elevated temperature for an effective dwell time, to purify the compressed air by deactivating one or more of, the one or more biological agents, and the one or more chemical agents, present in the compressed air, to obtain the purified air;
decompressing, with the at least one decompressor, the purified air, to cool the purified air to a cooled temperature;
supplying, with the air supply duct assembly, the purified air from the at least one compressor assembly to the one or more interior airflow spaces; and
returning, with the air return duct assembly, the recirculated air from the one or more interior airflow spaces to the at least one compressor assembly,
wherein the air purification and management system provides the contamination mitigation for the occupants of the one or more buildings against contamination by the one or more biological agents, and the one or more chemical agents.

16. The method of claim 15, wherein installing the air purification and management system at the one or more buildings, further comprises one of:
installing and retrofitting the air purification and management system at one or more retrofitted buildings, the air purification and management system installed and retrofitted in the one or more retrofitted buildings further comprises the air management system having external duct networks of ducts positioned external to the one or more interior airflow spaces; or
installing the air purification and management system at one or more new build buildings.

17. The method of claim 15, wherein installing the air purification and management system at the one or more buildings, further comprises installing the air purification and management system further comprising the at least one compressor assembly comprising a reciprocating compressor assembly further comprising:
the at least one compressor having a compression chamber with a compressor piston coupled to a compressor crankshaft, and the compressor crankshaft coupled to an electric motor;
the at least one plenum coupled to the at least one compressor, the at least one plenum comprising a vacuum-jacketed plenum tank, and the plenum inlet coupled to the compression chamber; and
the at least one decompressor coupled to the plenum exit, the at least one decompressor having a decompression chamber with a decompressor piston coupled to a decompressor crankshaft, and the decompressor crankshaft coupled to a generator.

18. The method of claim 15, wherein installing the air purification and management system at the one or more buildings, further comprises installing the air purification and management system further comprising the at least one compressor assembly comprising a multi-cylinder compressor assembly further comprising:
the at least one compressor having a plurality of intake cylinders coupled to a plurality of intake pipes, each intake cylinder having an intake cylinder piston coupled to an intake cylinder crankshaft, which is coupled to a first axle, the first axle coupled to a drive train, and the drive train coupled to a motor;
the at least one plenum comprising a plenum tank with a plurality of plenum inlets and a plurality of plenum exits, the plurality of plenum inlets coupled to the plurality of intake pipes; and
the at least one decompressor having a plurality of exit pipes coupled to the plurality of plenum exits of the plenum tank, and having a plurality of exit cylinders coupled to the plurality of exit pipes, each exit cylinder having an exit cylinder piston coupled to an exit cylinder crankshaft, which is coupled to a second axle, the second axle coupled to the drive train, and the drive train coupled to the motor.

19. The method of claim 15, wherein installing the air purification and management system at the one or more buildings, further comprises installing the air purification and management system further comprising the at least one compressor assembly comprising a generator turbine compressor assembly further comprising:
the at least one compressor comprising a multi-stage compressor coupled to a gearbox, which is coupled to an electric motor;
the at least one plenum coupled to the multi-stage compressor, the at least one plenum comprising a plenum tank; and
the at least one decompressor having a nozzle coupled to the plenum exit, and having a generator coupled to the nozzle.

20. The method of claim 15, wherein maintaining, with the at least one plenum, the compressed air at the elevated temperature for the effective dwell time, and purifying the compressed air by deactivating the one or more biological agents, and the one or more chemical agents, further comprises, one or more of:
deactivating the one or more biological agents comprising one or more:
airborne biological agents, airborne pathogens, respiratory pathogens, airborne viruses, respiratory viruses, airborne microbes, respiratory microbes, airborne coronaviruses, including SARS-CoV-2 (severe acute respiratory syndrome coronavirus 2), COVID-19 (coronavirus disease 2019), MERS (Middle East respiratory syndrome), ebola virus, Zika virus, West Nile virus, Marburg virus, influenza virus, including, airborne influenza virus, influenza A virus, influenza B virus, influenza C virus, avian flu, and swine flu, smallpox virus, monkeypox virus,Streptococcus pneumoniae causing pneumonia,Mycobacterium tuberculosis causing tuberculosis, Bacillus anthracis causing anthrax, aspergillus fumigatus causing lung aspergillosis, pneumonic plague, black mold airborne spores, ricin mist, and ricin powder; or
deactivating the one or more chemical agents comprising one or more:
airborne chemical agents, respiratory chemical agents, phenoxy herbicide, choking agents including chlorine, chloropicrin, diphosgene, and phosgene; blister agents including sulfur mustard, nitrogen mustard, lewisite, and phosgene oxime; blood agents including hydrogen cyanide, cyanogen chloride, and arsine; and nerve agents including tabun, sarin, soman, cyclosarin, and venomous agent X.
